(19) United States
(12) Patent Application Publication
(10) Pub. No.: US 2026/0102372 A1
(43) Pub. Date: Apr. 16, 2026

(54) NEW FORMULATIONS FOR MENTAL DISORDERS OR MENTAL ENHANCEMENT

(71) Applicant: TACTOGEN INC, Palo Alto, CA (US)

(72) Inventor: Matthew J. Baggott, Palo Alto, CA (US)

(73) Assignee: TACTOGEN INC, Palo Alto, CA (US)

(21) Appl. No.: 19/420,234

(22) Filed: Dec. 15, 2025

Related U.S. Application Data
(63) Continuation of application No. PCT/US2024/034161, filed on Jun. 14, 2024.
(60) Provisional application No. 63/521,300, filed on Jun. 15, 2023.

(51) Int. Cl. A61K 31/36; A61K 31/138; A61K 31/165; A61K 31/343; A61K 31/381; A61K 31/4525

(52) U.S. Cl. CPC A61K 31/36; A61K 31/165; A61K 31/381; A61K 31/138; A61K 31/343; A61K 31/4525

(57) ABSTRACT

New pharmaceutical formulations are provided comprising a specific entactogen and a selective serotonin reuptake inhibitor (SSRI) or a serotonin and norepinephrine reuptake inhibitor (SNRI) and methods of use and manufacture thereof, for the treatment of central nervous system (CNS) disorders, including a mental disorder, or to provide mental enhancement.

### CROSS-REFERENCE TO RELATED APPLICATIONS

This application is a continuation of International Patent Application No. PCT/US2024/034161, filed in the U.S. Receiving Office on Jun. 14, 2024, which claims the benefit of U.S. Provisional Application 63/521,300, filed on Jun. 15, 2023. The entirety of each of these applications is hereby incorporated by reference for all purposes.

### FIELD OF INVENTION

New pharmaceutical formulations are provided comprising a specific entactogen and a selective serotonin reuptake inhibitor (SSRI) or a serotonin and norepinephrine reuptake inhibitor (SNRI) and methods of use and manufacture thereof, for the treatment of central nervous system (CNS) disorders, including a mental disorder, or to provide mental enhancement.

### BACKGROUND

Central nervous system (CNS) related health problems are a common challenge in society. An estimated 20.6% of U.S. adults (51.5 million people) experienced mental illness in 2019. This includes major depression (7.8% or 19.4 million people), anxiety disorders (19.1% or 48 million people), and posttraumatic stress disorder (PTSD) (3.6% or 9 million people). In addition to mental health challenges, there are other CNS disorders that cause substantial suffering and decreased quality of life. These include traumatic brain injury (TBI) (an estimated 12% of adults or 30 million people in the U.S.), dementias, and headache disorders (such as migraine, which affects about 15% of the general population or 47 million people in the U.S.). As the global population ages, many age-related CNS disorders are projected to become more common. For example, 6.2 million people aged 65 and older in the U.S. have Alzheimer's dementia and this population is expected to grow to 12.7 million by 2050.

There is a need for improved treatment of CNS disorders. Many patients fail to benefit adequately from available treatments. In addition, many available pharmacological treatments must be taken for weeks or months before the individual experiences therapeutic benefits. Because of these and other considerations, fewer than half of U.S. adults with mental illness (44.8%) received treatment in 2019.

Entactogens (sometimes called empathogens) have become the focus of attention as a tool to help solve some of these serious health problems. They increase feelings of authenticity and emotional openness while decreasing social anxiety (Baggott et al., Journal of Psychopharmacology 2016, 30.4: 378-87). Entactogens have potential for cultivating intimacy, open communication, and interpersonal healing in human relationships. This is in part because they seem to allow individuals to engage in emotionally meaningful activity with lessened influence from trauma history, attachment patterns, and self-criticism.

Entactogens are typically monoamine releasers that appear to produce their effects in part by increasing extracellular serotonin in the brain, which both stimulates hypothalamic serotonergic receptors, thus triggering release of the hormone oxytocin, and also stimulates serotonergic 5-HT_{1B} receptors on cells in the nucleus accumbens area of the brain. However, these drugs do have varying and complex effects that result from binding to a range of 5-HT and other receptors. Entactogens can be distinguished from drugs that are primarily hallucinogenic or psychedelic, and amphetamines, which are primarily stimulants.

The most well-known entactogen, MDMA (3,4-methylenedioxymethamphetamine), is currently in human clinical trials in the United States and Europe. It is being tested as an aid to psychotherapy sessions for PTSD and has been more broadly suggested as useful for aiding social cognition (Preller & Vollenweider, Frontiers in Psychiatry, 2019, 10; Hysek et al., Social cognitive and affective neuroscience, 2015, 9.11, 1645-52). The FDA granted breakthrough therapy designation for the PTSD program and has also agreed to an expanded access program, both indicative of promising results. (Feduccia et al., Frontiers in Psychiatry, 2019, 10: 650; Sessa et al., Frontiers in Psychiatry, 2019, 10: 138; see also the MDMA Investigator's Brochure, 14th Edition: Mar. 18, 2022, and references therein, available from the sponsor of MDMA clinical trials at MAPS.org). Indeed, in the first of two Phase 3 trials using MDMA-assisted therapy to treat PTSD, 88% of participants in the MDMA arm improved and one-third of participants in the MDMA arm had sufficient improvement that they were no longer diagnosable with PTSD (Mitchell et al. 2021. Nature Medicine. 27(6):1025-33).

Although MDMA appears to have significant therapeutic value, it has some features that reduce its acceptability to patients and may limit its clinical uses. For example, many patients find that the initial effects are moderately unpleasant. These initial effects can include nausea (sometimes including vomiting) and anxiety. In a pooled analysis of Phase 2 clinical trials, nausea occurred in 40.3% (29 of 72) of participants, while anxiety occurred in 72.2% (52 of 72) of participants (Table 6 in Mithoefer et al. 2019. Psychopharmacology. 236(9):2735-45). Additionally, MDMA can produce feelings of dizziness, sedation, drunkenness, difficulty concentrating, and mild confusion during the first five hours after drug administration. These and other undesired effects can distract the patient from both the therapeutic effects of the drug and psychotherapy or other activities taking place during the hours after administering MDMA.

There is also considerable variability in the effects of MDMA. The acute psychological and emotional effects of MDMA have a duration reported as averaging 4.2 hours with a standard deviation of 1.3 hours after 75 or 125 mg MDMA by Vizeli & Liechti (2017. Journal of Psychopharmacology, 31(5), 576-588). Part of the variability in duration is likely due to relatively high inter-person variation in the pharmacokinetics of MDMA and metabolites such as HMMA (4-hydroxy-3-methoxymethamphetamine) (Kolbrich et al. 2008. Therapeutic drug monitoring. 30(3):320). Some of this variability in kinetics may be because MDMA is both a substrate and inhibitor of cytochrome p450 isozyme 2D6 (CYP2D6).

Patents and patent applications describing entactogenic compounds include WO 2021/252538, WO 2022/010937, WO 2022/032147, WO 2022/061242, WO 2023/081306, WO 2023/107653, WO 2023/107715, WO 2023/183613, and U.S. Pat. No. 11,767,305, which are assigned to Tactogen Inc. Additional patents and patent applications describing entactogenic compounds and methods of using entactogenic compounds include but are not limited to U.S. Pat. Nos. 7,045,545, 11,603,353, US2023/233688, WO 2005/058865, WO 2020/169850, WO 2020/169851, WO 2021/257169, WO 2021/225796, WO2022/106947, WO 2022/214889, WO 2022/120181, WO 2022/072808, WO 2022/038171, WO 2023/049480, WO 2023/036473, WO 2023/028022, WO 2023/028092, and WO 2023/028091.

It is an object of the present invention to provide advantageous compositions and their use and manufacture for improving the therapeutic effects of entactogens by decreasing undesired side effects.

### SUMMARY OF THE INVENTION

The present invention provides new engineered formulations that decrease the side effects of specific entactogenic compounds such as MDMA by providing in combination a serotonin reuptake inhibitor (SSRI) or serotonin and norepinephrine reuptake inhibitor (SNRI) that has a delayed release relative to the entactogen. According to the invention, the SSRI or SNRI is released in a delayed manner that minimizes or eliminates the activity of the entactogenic compound. By providing this engineered formulation, the desired therapeutic effect of the entactogenic compound can be achieved and then the activity of the entactogen can be stopped in a more deliberate fashion than has previously been available. These new engineered formulations provide more control over CNS therapy, including psychotherapy and mental enhancement, and allow the patient to return to his or her normal state with a more intentional time frame.

These methods and preparations are based on the finding that the pharmacological and therapeutic profiles of entactogens and related drugs can be improved by changing the timing by which increases of different brain monoamines occur. To accomplish the desired timing, in certain aspects, a pharmaceutical composition is provided comprising granules formulated for immediate release with an entactogen and granules formulated for delayed or controlled release with an SSRI or SNRI wherein the granules achieve varied release profiles by selection of excipients or by use of coatings for example an enteric coating on the delayed release granules. In other aspects the desired timing is accomplished by using a bilayer tablet, trilayer tablet, multi compartment tablet such as a two-compartment tablet, a capsule comprising microparticles, nanoparticles, or lipid nanoparticles or a combination of a tablet structure and delayed/immediate release granules described herein.

For example, in certain embodiments a bilayer tablet is provided comprising an inner and outer layer wherein the inner layer comprises (1) delayed release granules comprising a selective serotonin reuptake inhibitor (SSRI) or a serotonin and norepinephrine reuptake inhibitor (SNRI) and one or more pharmaceutically acceptable excipients; and (2) optionally one or more additional pharmaceutically acceptable excipients wherein the granules are dispersed therein; and wherein the outer layer comprises (1) immediate release granules comprising an entactogen; and (2) optionally one or more pharmaceutically acceptable excipients that the immediate release granules are dispersed in. The dispersion excipients of (2) can be the same or different. In some embodiments, the delayed release and immediate release granules are in the same layer, for example a bilayer tablet is provided comprising an inner and outer layer wherein the inner layer comprises (1) immediate release granules comprising an entactogen and one or more pharmaceutically acceptable excipients; (2) delayed release granules comprising a selective serotonin reuptake inhibitor (SSRI) or a serotonin and norepinephrine reuptake inhibitor (SNRI) and one or more pharmaceutically acceptable excipients; and (3) one or more additional pharmaceutically acceptable excipients; wherein the outer layer is a film coating.

In certain aspects a formulation is provided which delivers (1) an entactogen selected from MDMA, MDA, MDE, MBDB, BDB, MDAI, MDMAI, MDAT, MDMAT, BK-MDMA, BK-MDEA, 5-APB, 6-APB, 5-MAPB, 6-MAPB, BK-5-MAPB, BK-5-EAPB, 5-MAPBT, 6-MAPBT, 5-EAPB, 6-EAPB, 5-MBPB, 6-MBPB, 5-MBPBT, 6-MBPBT, 5-EAPBT, and 6-EAPBT, or a pharmaceutically acceptable salt thereof, and (2) a SSRI or SNRI wherein the SSRI is a compound selected from citalopram, escitalopram, fluoxetine, fluvoxamine, paroxetine, sertraline, vortioxetine, and vilazodone, or a pharmaceutically acceptable salt thereof, and the SNRI is a compound selected from desvenlafaxine, duloxetine, levomilnacipran, milnacipran, and venlafaxine, or a pharmaceutically acceptable salt thereof, wherein the peak effects of the SSRI or SNRI are delayed compared to the peak effects of the entactogen.

In certain aspects the entactogen is enantiomerically enriched. For example, in certain embodiments the entactogen is an enantiomerically enriched mixture with between 55% and 95% S-enantiomer. In other embodiments the entactogen is an enantiomerically enriched mixture with between 55% and 95% R-enantiomer. Non-limiting examples of entactogens for use in the present invention include an enantiomerically enriched mixture of 5-MAPB, 6-MAPB, 5-MBPB, 6MBPB, Bk-5-MAPB, Bk-6-MAPB, Bk-5-MBPB, or Bk-6-MBPB.

In certain embodiments the formulation comprises about 10-180 mg of MDMA and about 10-100 mg of milnacipran wherein the peak effects of milnacipran are delayed in a manner that allows the patient to experience the entactogenic effect of MDMA and then have an intentional recovery back to a normal state. In other embodiments the formulation comprises about 10-180 mg of MDMA and about 20-40 mg of citalopram wherein the peak effects of citalopram are delayed in a manner that allows the patient to experience the entactogenic effect of MDMA and then have an intentional recovery back to a normal state. Additional examples of formulations of the present invention include formulations comprising about 10-180 mg of MDMA and about 20-40 mg of escitalopram or about 20-60 mg of duloxetine wherein the peak effects of escitalopram or duloxetine are delayed in a manner that allows the patient to experience the entactogenic effect of MDMA and then have an intentional recovery back to a normal state.

Surprisingly, although both entactogens and SSRIs/SNRIs increase extracellular concentrations of monoamines, the two classes of compounds do not have additive effects. Both classes work by interacting with monoamine transporters on the membranes of neurons. However, they work in incompatible ways, such that SSRIs/SNRIs block the ability of entactogens to interact with the serotonin transporter (in the case of SSRIs) or with both the serotonin and norepinephrine transporters (in the case of SNRIs). The blocking of entactogen interactions by SSRIs/SNRIs is concentration dependent. Yet because the SSRIs/SNRIs for use in the present invention are selected to have high affinity for these transporters, these SSRIs/SNRIs outcompete entactogens for interactions with the transporters.

It is well documented that tachyphylaxis (rapid tolerance) develops to the many of the effects of MDMA and some common entactogens. As a result, concentrations of entactogens in the blood and other parts of the body typically decrease slower than acute emotional effects. For example, the acute emotional effects of MDMA have largely resolved by 8 hours after administration of MDMA. In contrast, the half-life of MDMA in plasma is seven to nine hours. Thus, MDMA continues to have high concentrations in the body and produces pharmacological and toxic effects well after the acute emotional effects have resolved. In nonhuman animals, MDMA causes unwanted increases in measures of oxidative stress and unwanted decreases in markers of serotonergic functioning (e.g., brain serotonin concentrations, brain levels and activity of tryptophan hydroxylase, and density of serotonin transporter in the brain). Controlled release of an SSRI/SNRI can decrease these unwanted effects and others and can be lagged from release of the entactogen to allow a time period during which there are emotional and other effects of MDMA that are clinically useful.

The formulations and methods of the present invention allow the patient to feel the effects of the entactogen first and then subsequently the effects of the SSRI or SNRI are felt. In certain embodiments the effects of the SSRI or SNRI decrease or halt the effects of the entactogen allowing the formulation to be administered in a convenient time period for psychotherapy. In certain aspects these new formulations also exhibit decreased abuse liability.

In certain embodiments there is a kinetic lag between the maximum or peak effect of the entactogen and the maximum or peak effect of the SSRI or SNRI. In certain embodiments the kinetic lag is a later Tmax of the SSRI or SNRI than the entactogen. For example, the Tmax of a SSRI or SNRI delivered by formulations of the present invention may be about or between 0.5, 1, 1.5, 2, 3, 4, 5, 6, 7, or 8 hours (for example, but not limited to between 0.5 and 8 hours, between 1 and 8 hours, between 1 and 7 hours, 2 and 6 hours, 2 and 5 hours, or 2 and 4 hours) later than the entactogen. In certain embodiments the kinetic lag is a later Cmax or 50% Cmax of the SSRI or SNRI than the entactogen for example a SSRI or SNRI may have a Cmax or 50% Cmax that is at least is about 0.5, 1, 1.5, 2, 3, 4, 5, 6, 7, or 8 hours (for example, but not limited to, between 0.5 and 8 hours, between 1 and 8 hours, between 1 and 7 hours, 2 and 6 hours, 2 and 5 hours, or 2 and 4 hours), later than the entactogen.

This kinetic lag is achieved by using an oral dosage form with a delayed release component as discussed in further detail below.

In certain embodiments, the kinetic lag shortens the duration of acute effects of the entactogen, reducing the time that it is necessary to monitor the patient. In some embodiments, these acute effects are measured in humans and are any of (or a combination of): increases in blood pressure, heart rate, body temperature, subjective feelings of drug effects, including anxiety, stimulation, sedation, good drug effects, bad drug effects, or any drug effects. In some embodiments, these acute effects are measured in nonhuman animals and are any of: blood pressure, heart rate, body temperature, or locomotor activity.

In certain embodiments, the lag decreases adverse subacute effects of the entactogen, which can be understood as any effects occurring between about 6 and about 72 hours or more after administration of an entactogen. In some embodiments, these subacute effects are measured in humans and are any of: worsened concentration, attention, working memory, inhibition, or other aspects of cognitive performance (including impairment in standard neurocognitive tasks, such as the Continuous Performance Task or Stop Signal Task), insomnia, subjective feelings of drug effects, including anxiety or panic, difficulty sleeping, tiredness, muscle aches or soreness, headache, lack of appetite, lack of energy, dry mouth, difficulty concentrating, bruxism, stimulation, sedation, bad drug effects, or any drug effects. In some embodiments, these subacute effects are decreases in measures of serotonergic tone, including brain serotonin, brain serotonin transporter, or brain tryptophan hydroxylase. In some embodiments these measures are made in nonhuman animals; in other embodiments, these measures are made in humans using radiolabeled ligands and SPECT, PET, or another imaging technique.

In some embodiments, the formulation has reduced abuse liability, because the entactogenic effect is moderated. In some embodiments, parenteral administration of the active ingredients results in an attenuation of entactogen effects. In some embodiments, this reduced abuse liability is measured in humans using ratings of willingness to pay, drug liking, good drug effects, or general measures of entactogen effects. In some embodiments, this reduced abuse liability is measured in nonhumans as decreased dose self-administered per session, increased ED₅₀ for drug discrimination, decreased conditioned place preference, or decreased drug-induced locomotor activity. These comparisons can be made between a preparation with the entactogen alone and a preparation with the entactogen and SSRI/SNRI. Other well-known measures of abuse liability are also contemplated.

In certain aspects the formulation comprises an entactogen combined with an SSRI. In certain embodiments the SSRI for use in the present invention is citalopram, escitalopram, sertraline, fluoxetine, paroxetine, fluvoxamine, vilazodone, or vortioxetine.

In certain aspects the formulation comprises an entactogen combined with an SSRI. In certain embodiments the SNRI for use in the present invention is duloxetine, milnacipran, levomilnacipran, venlafaxine, or desvenlafaxine.

Non-limiting examples of a formulation comprising specialized combinations of the present invention include:
i. 10-180 mg of RS-MDMA, or nonracemic MDMA, formulated for immediate release, and 5-40 mg of citalopram formulated for delayed release;
ii. 10-180 mg of RS-MDMA, or nonracemic MDMA, formulated for immediate release and 5-30 mg of escitalopram formulated for delayed release;
iii. 10-180 mg of RS-MDMA, or nonracemic MDMA, formulated for immediate release, and 10-80 mg of fluoxetine formulated for delayed release;
iv. 10-180 mg of RS-MDMA, or nonracemic MDMA, formulated for immediate release, and 10-60 mg of paroxetine formulated for delayed release;
v. 10-180 mg of RS-MDMA, or nonracemic MDMA, formulated for immediate release, and 10-120 mg of duloxetine formulated for delayed release;
vi. 10-180 mg of RS-MDMA, or nonracemic MDMA, formulated for immediate release, and 5-300 mg of milnacipran formulated for delayed release;
vii. 20-160 mg of RS-5-MAPB, or nonracemic 5-MAPB, formulated for immediate release, and 5-40 mg of citalopram formulated for delayed release;
viii. 20-160 mg of RS-5-MAPB, or nonracemic 5-MAPB, formulated for immediate release, and 5-30 mg of escitalopram formulated for delayed release;
ix. 20-160 mg of RS-5-MAPB, or nonracemic 5-MAPB, formulated for immediate release, and 10-80 mg of fluoxetine formulated for delayed release;
x. 20-160 mg of RS-5-MAPB, or nonracemic 5-MAPB, formulated for immediate release, and 10-60 mg of paroxetine formulated for delayed release;
xi. 20-160 mg of RS-5-MAPB, or nonracemic 5-MAPB, formulated for immediate release, and 10-120 mg of duloxetine formulated for delayed release;
xii. 20-160 mg of RS-5-MAPB, or nonracemic 5-MAPB, formulated for immediate release, and 5-300 mg of milnacipran formulated for delayed release;
xiii. 20-200 mg of RS-MDA, or nonracemic MDA, formulated for immediate release, and 5-40 mg of citalopram formulated for delayed release;
xiv. 20-200 mg of RS-MDA, or nonracemic MDA, formulated for immediate release, and 5-30 mg of escitalopram formulated for delayed release;
xv. 20-200 mg of RS-MDA, or nonracemic MDA, formulated for immediate release, and 10-80 mg of fluoxetine formulated for delayed release;
xvi. 20-200 mg of RS-MDA, or nonracemic MDA, formulated for immediate release, and 10-60 mg of paroxetine formulated for delayed release;
xvii. 20-200 mg of RS-MDA, or nonracemic MDA, formulated for immediate release, and 10-120 mg of duloxetine formulated for delayed release; and
xviii. 20-200 mg of RS-MDA, or nonracemic MDA, formulated for immediate release, and 5-300 mg of milnacipran formulated for delayed release.

In certain embodiments the formulation comprises an entactogen combined with a SSRI or SNRI and is used for a single dose therapy wherein one dose of the formulation of the present invention is administered before or during the therapy session.

In other aspects a formulation is provided comprising a mixture of an entactogen and an SSRI or SNRI as disclosed in an embodiment herein, for example any one of embodiments i. to xviii. above, wherein the entactogen, SSRI, and/or SNRI are pharmaceutically acceptable salts and the dose refers to the dose of the active agent without considering the weight of the salt. In alternative embodiments the dose refers to the amount of the active agent with the weight of the salt included.

In certain embodiments a two-dose therapy is used wherein the entactogen is administered by itself followed by administration of a formulation of the present invention comprising the entactogen and an SSRI or SNRI about 1, 2, 3, 4, 5, or 6 hours later. In certain embodiments the formulation of the present invention comprises a lower dose of entactogen than the first dose administered. In other embodiments the two doses comprise the same amount of entactogen. For example, in certain embodiments a two-dose therapy is used wherein RS-MDMA is administered, followed by administration of a RS-MDMA+citalopram formulation about 1, 2, 3, 4, 5, or 6 hours later. In certain other embodiments a two-dose therapy is used wherein RS-MDMA is administered, followed by administration of a RS-MDMA+duloxetine formulation about 1, 2, 3, 4, 5, or 6 hours later.

The invention includes methods for modulating the activity of the CNS of a host in need thereof, such as a human, by administering an effective amount of a formulation of the invention. Examples are methods for treating a CNS disorder, as generally listed herein, that have been linked to inadequate functioning of serotonergic neurotransmission in mammals, using a formulation of the invention. The invention also includes methods of improving CNS functioning such as reducing neuroticism or psychological defensiveness or increasing creativity, decision-making ability, or openness to experience in a human by administering an effective amount of a formulation of the invention. In certain embodiments the combination or pharmaceutical formulation of the present invention is used to treat an alcohol use disorder.

Specifically, the invention includes methods to treat a neurological or psychiatric central nervous system disorder as further described herein, including a mental disorder, or to provide a mental enhancement using a formulation of the invention.

The present invention thus includes at least the following aspects:
(i) A formulation (which is a coformulation) comprising an entactogen, a selective serotonin reuptake inhibitor (SSRI) or a serotonin and norepinephrine reuptake inhibitor (SNRI) and one or more pharmaceutically acceptable excipients,
(ii) The formulation of (i) when the entactogen is formulated for an immediate release and SSRI is formulated for a delayed release in a manner that allows the patient to experience the entactogenic effect of MDMA and then have an intentional recovery back to a normal state,
(iii) The formulation of (i) when the entactogen is formulated for an immediate release and the SNRI is formulated for a delayed release in a manner that allows the patient to experience the entactogenic effect of MDMA and then have an intentional recovery back to a normal state,
(iv) The formulation of (i) where the entactogen is formulated as a racemic mixture (RS),
(v) The formulation of (i) where the entactogen is formulated as enantiomerically enriched or pure R-enantiomer,
(vi) The formulation of (i) where the entactogen is formulated as enantiomerically enriched or pure S-enantiomer,
(vii) The formulation of any one of (i)-(vi) where the kinetic lag between the entactogen and the SSRI or SNRI is two to eight hours,
(viii) A method for treating a patient with a neurological or psychological CNS disorder described herein that includes administering an effective amount of a formulation of (i)-(vii) to a patient such as a human in need thereof,
(ix) Use of a formulation of (i)-(vii) to treat a disorder described herein;
(x) A processes for the preparation of a therapeutic product such as a tablet comprising a formulation of (i)-(vii), as described herein.

### DETAILED DESCRIPTION

The present invention provides new engineered formulations that decrease the side effects of specific entactogenic compounds such as MDMA by providing a serotonin reuptake inhibitor (SSRI) or serotonin and norepinephrine reuptake inhibitor (SNRI) with a delayed release as a co-formulated dosage form. According to the invention, the SSRI or SNRI is released in a delayed manner that minimizes or eliminates the activity of the entactogenic compound. By providing this engineered formulation, the desired therapeutic effect of the entactogenic compound can be achieved and then the activity of the entactogen can be stopped in a more deliberate fashion than has previously been available. These new engineered formulations provide more control over CNS therapy, including psychotherapy, and mental enhancement, and allow the patient to return to his or her normal state with a more intentional time frame.

The present invention includes new combinations and methods for administering entactogens that can increase the proportion of desired effects compared to undesired effects. These methods and preparations are based on the finding that the pharmacological and therapeutic profiles of entactogens can be improved by coadministration of an SSRI or SNRI as they decrease or halt the effects of the entactogen allowing the formulation to be administered in a convenient time period for psychotherapy and then stopped.

The embodiments of the invention are presented to meet the goal of assisting persons with mental disorders, who desire mental enhancement, or who suffer from other CNS disorders by providing mild therapeutics that are fast acting and that reduce the properties that decrease the patient experience, are counterproductive to the therapy, or are undesirably toxic. One goal of the invention is to provide therapeutic compositions that increase empathy, sympathy, openness and acceptance of oneself and others, which can be taken, if necessary, as part of therapeutic counseling sessions, when necessary, episodically or even consistently, as prescribed by a healthcare provider.

### Definitions

When introducing elements of the present invention or the preferred embodiments thereof, the articles “a,” “an,” “the,” and “said” are intended to mean that there are one or more of the elements. The terms “comprising,” “including,” and “having” are intended to be inclusive and not exclusive (i.e., there may be other elements in addition to the recited elements). Thus, the terms “including,” “may include,” and “include,” as used herein mean, and are used interchangeably with, the phrase “including but not limited to.”

Where a range of values is provided, it is understood that the upper and lower limit, and each intervening value between the upper and lower limit of the range is encompassed within the embodiments.

Unless defined otherwise, all technical and scientific terms herein have the meaning as commonly understood by one of ordinary skill in the art to which this invention belongs. In the event there is a plurality of definitions for a term herein, those in this section prevail unless stated otherwise. Further definitions that may assist the reader to understand the disclosed embodiments are as follows, and such definitions may be used to interpret the defined terms, when those terms are used herein. However, the examples given in the definitions are generally non-exhaustive and must not be construed as limiting the invention. It also will be understood that a substituent should comply with chemical bonding rules and steric compatibility constraints in relation to the particular molecule to which it is attached.

Entactogen as defined herein is a compound selected from MDMA ([1-(2H-1,3-benzodioxol-5-yl)propan-2-yl](methyl)amine), MDA (1-(2H-1,3-benzodioxol-5-yl)propan-2-amine), MDE ([1-(2H-1,3-benzodioxol-5-yl)propan-2-yl](ethyl)amine), MBDB ([1-(2H-1,3-benzodioxol-5-yl)butan-2-yl](methyl)amine), BDB (1-(2H-1,3-benzodioxol-5-yl)butan-2-amine), MDAI (2H,5H,6H,7H-indeno[5,6-d][1,3]dioxol-6-amine), MDMAI (N-methyl-2H,5H,6H,7H-indeno[5,6-d][1,3]dioxol-6-amine), MDAT (2H,5H,6H,7H,8H-naphtho[2,3-d][1,3]dioxol-6-amine), MDMAT (N-methyl-2H,5H,6H,7H,8H-naphtho[2,3-d][1,3]dioxol-6-amine), BK-MDMA (MDMC, methylone, 1-(2H-1,3-benzodioxol-5-yl)-2-(methylamino)propan-1-one), BK-MDEA (MDEC, ethylone, 1-(2H-1,3-benzodioxol-5-yl)-2-(ethylamino)propan-1-one), 5-APB (1-(1-benzofuran-5-yl)propan-2-amine), 6-APB (1-(1-benzofuran-6-yl)propan-2-amine), 5-MAPB ([1-(1-benzofuran-5-yl)propan-2-yl](methyl)amine), 6-MAPB ([1-(1-benzofuran-6-yl)propan-2-yl](methyl)amine), BK-5-MAPB (1-(1-benzofuran-5-yl)-2-(methylamino)propan-1-one), BK-5-EAPB (1-(1-benzofuran-5-yl)-2-(ethylamino)propan-1-one), 5-MAPBT ([1-(1-benzothiophen-5-yl)propan-2-yl](methyl)amine), 6-MAPBT ([1-(1-benzothiophen-6-yl)propan-2-yl](methyl)amine), 5-EAPB ([1-(1-benzofuran-5-yl)propan-2-yl](ethyl)amine), 6-EAPB ([1-(1-benzofuran-6-yl)propan-2-yl](ethyl)amine), 5-MBPB ([1-(1-benzofuran-5-yl)butan-2-yl](methyl)amine), 6-MBPB ([1-(1-benzofuran-6-yl)butan-2-yl](methyl)amine), 5-MBPBT ([1-(1-benzothiophen-5-yl)butan-2-yl](methyl)amine), 6-MBPBT ([1-(1-benzothiophen-6-yl)butan-2-yl](methyl)amine), 5-EAPBT ([1-(1-benzothiophen-5-yl)propan-2-yl](ethyl)amine), and 6-EAPBT ([1-(1-benzothiophen-6-yl)propan-2-yl](ethyl)amine).

In alternative aspects the entactogen is a halogenated benzofuran of formula:

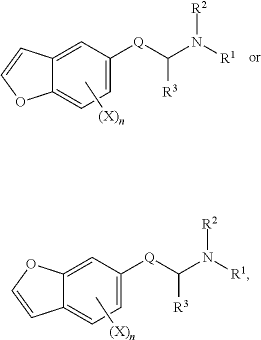

or a pharmaceutically acceptable salt or salt mixture thereof;
wherein:
R¹ and R² are independently selected from H, C₁-C₄ alkyl, C₁-C₄ haloalkyl, and —CH₂CH₂OH;
R³ is selected from H, C₁-C₄ alkyl, C₁-C₄ haloalkyl, CH₂OH, and —CH₂CH₂OH;
in certain embodiments R³ is C₁-C₂ alkyl;
Q is

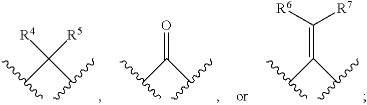

R⁴ and R⁵ are independently selected from H, C₁-C₄ alkyl, C₁-C₄ haloalkyl, F, Cl, and Br;
R⁶ and R⁷ are independently selected from H, C₁-C₄ alkyl, C₁-C₄ haloalkyl, F, Cl, and Br; each X is independently selected from —F, —Cl, and —Br; and
n is 1 or 2.

In certain embodiments each X is F.

In certain embodiments each Y is F.

In certain embodiments n is 1.

Non-limiting examples of a halogenated benzofuran compound include:

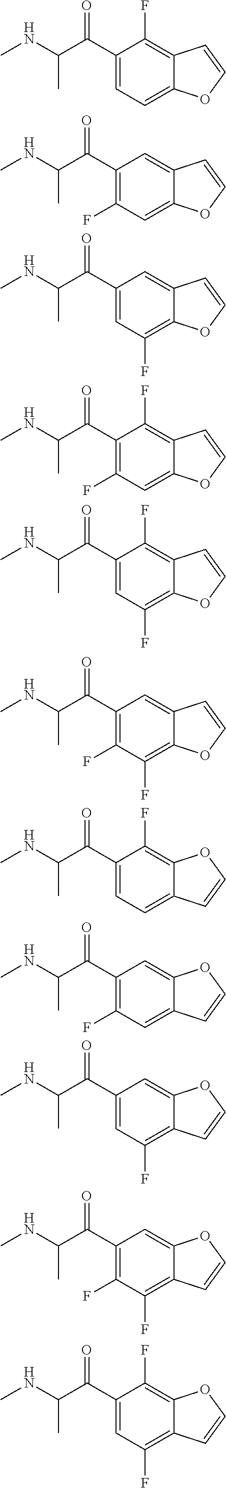

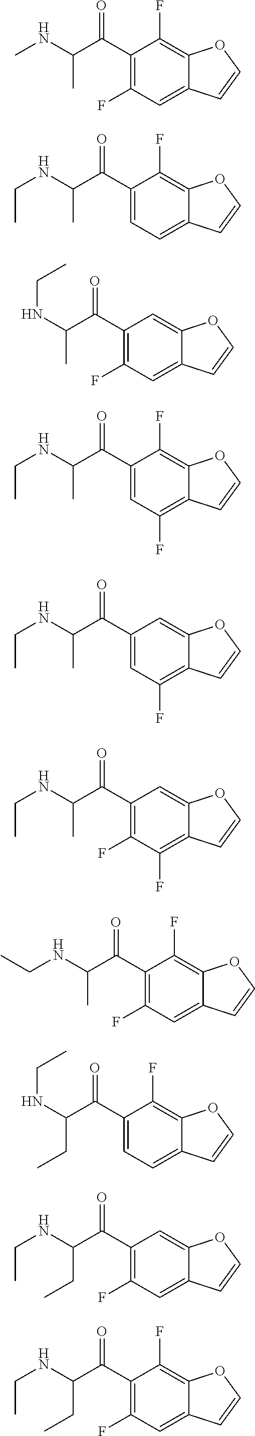

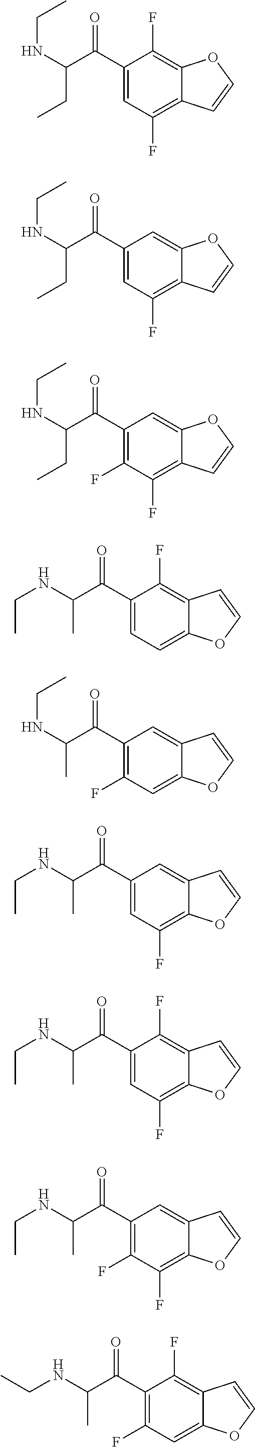

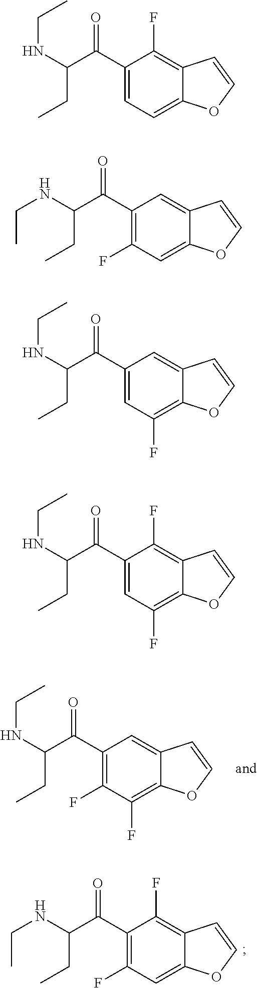 [SMILES: CCNC(CC)C(=O)c1ccc2occc2c1F ; CCNC(CC)C(=O)c1cc2ccoc2cc1F ; CCNC(CC)C(=O)c1cc(F)c2occc2c1 ; CCNC(CC)C(=O)c1cc(F)c2occc2c1F ; CCNC(CC)C(=O)c1cc2ccoc2c(F)c1F ; CCNC(CC)C(=O)c1c(F)cc2occc2c1F]

or a pharmaceutically acceptable salt thereof.

Additional non-limiting examples of halogenated benzofurans include:

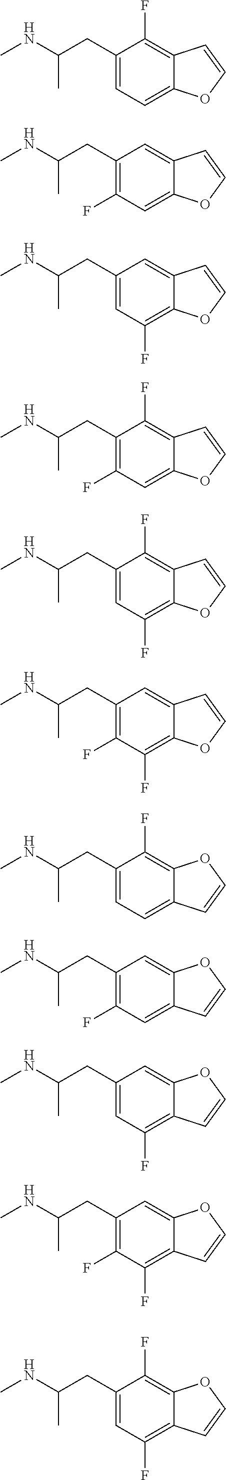

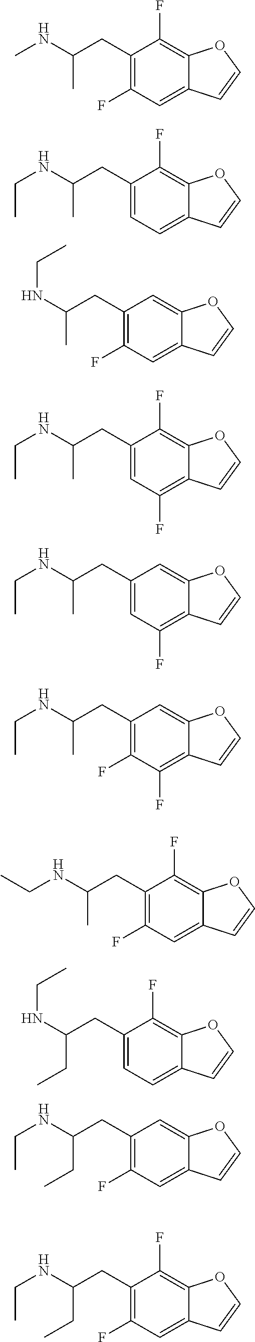

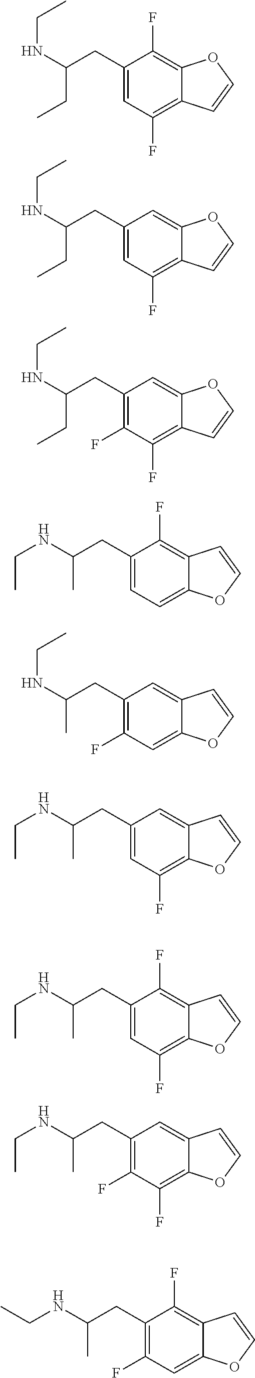

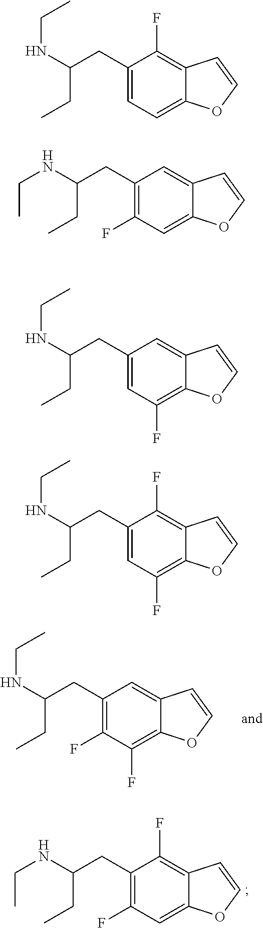

or a pharmaceutically acceptable salt thereof.

A selective serotonin reuptake inhibitor (SSRI) is a drug that increases the extracellular level of serotonin by limiting its reabsorption into the presynaptic cell. Non-limiting examples of SSRIs include citalopram, escitalopram, sertraline, fluoxetine, paroxetine, fluvoxamine, vilazodone, vortioxetine, femoxetine, litoxetine, McN 5652, DASB (3-amino-4-[2-[(di(methyl)amino)methyl]phenyl]sulfanylbenzonitrile), cyanodothepine, cericlamine, and SSRIs described in Singh, I. et al. Structure-based discovery of conformationally selective inhibitors of the serotonin transporter. Cell. 2023 May 11; 186(10):2160-2175.e17.

In certain embodiments the SSRI is a compound of structure

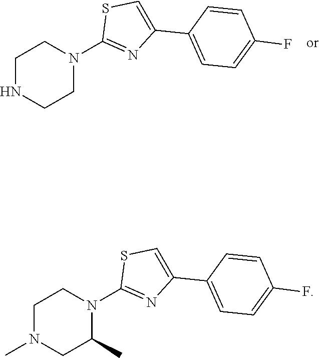 [SMILES: Fc1ccc(-c2csc(N3CCNCC3)n2)cc1 ; "or" ; C[C@H]1CN(C)CCN1c1nc(-c2ccc(F)cc2)cs1]

A serotonin-norepinephrine reuptake inhibitor (SNRI) is a drug that inhibits the reuptake of serotonin and norepinephrine. Non-limiting examples of SNRI include duloxetine, milnacipran, levomilnacipran, venlafaxine, desvenlafaxine, tesofensine, DOV 216303, and DOV 31947.

An enantiomerically enriched mixture is a mixture that contains one enantiomer in a greater amount than the other. In certain embodiments an enantiomerically enriched mixture of an S-enantiomer contains at least 55% of the S-enantiomer, and, typically at least about 60%, 65%, 70%, 75%, 80%, 85%, 90%, or 95% or more of the S-enantiomer and not more than 95%. In certain embodiments an enantiomerically enriched mixture of an R-enantiomer contains at least 55% of the R-enantiomer, and typically at least about 60%, 65%, 70%, 75%, 80%, 85%, 90% or 95% of the R-enantiomer and not more than 95%. The specific ratio of S or R enantiomer can be selected for the need of the patient according to the health care specialist to balance the desired effect. The term enantiomerically enriched mixture as used herein does not include either a racemic mixture or a substantially pure or pure enantiomer (greater than 99% or even essentially 100% pure).

In certain embodiments the enantiomerically enriched mixture contains at least or about 55% S-enantiomer. In certain embodiments the enantiomerically enriched mixture contains at least or about 60% S-enantiomer. In certain embodiments the enantiomerically enriched mixture contains at least or about 65% S-enantiomer. In certain embodiments the enantiomerically enriched mixture contains at least or about 70% S-enantiomer. In certain embodiments the enantiomerically enriched mixture contains at least or about 75% S-enantiomer. In certain embodiments the enantiomerically enriched mixture contains at least or about 80% S-enantiomer. In certain embodiments the enantiomerically enriched mixture contains at least or about 85% S-enantiomer. In certain embodiments the enantiomerically enriched mixture contains at least or about 90% S-enantiomer. In certain embodiments the enantiomerically enriched mixture contains about 95% S-enantiomer.

In certain embodiments the enantiomerically enriched mixture contains at least or about 55% R-enantiomer. In certain embodiments the enantiomerically enriched mixture contains at least or about 60% R-enantiomer. In certain embodiments the enantiomerically enriched mixture contains at least or about 65% R-enantiomer. In certain embodiments the enantiomerically enriched mixture contains at least or about 70% R-enantiomer. In certain embodiments the enantiomerically enriched mixture contains at least or about 75% R-enantiomer. In certain embodiments the enantiomerically enriched mixture contains at least or about 80% R-enantiomer. In certain embodiments the enantiomerically enriched mixture contains at least or about 85% R-enantiomer. In certain embodiments the enantiomerically enriched mixture contains at least or about 90% R-enantiomer. In certain embodiments the enantiomerically enriched mixture contains about 95% R-enantiomer.

In certain aspects the term “CNS disorder” as used herein refers to either a neurological condition (one that is typically treated by a neurologist) or a psychiatric condition (one that is typically treated by a psychiatrist). Neurological disorders are typically those affecting the structure, biochemistry or normal electrical functioning of the brain, spinal cord or other nerves. Psychiatric conditions are more typically thought of as mental disorders, which are primarily abnormalities of thought, feeling or behavior that cause significant distress or impairment of personal functioning. Thus, a disclosed composition may be used to improve neurological or psychiatric functioning in a patient in need thereof. Neurological indications include, but are not limited to improved neuroplasticity, including treatment of stroke, brain trauma, dementia, and neurodegenerative diseases. In certain embodiments a pharmaceutical composition of the present invention is a psychoplastogen. A psychoplastogen is a small molecule that induces rapid neuroplasticity and has rapid and sustained effects on neuronal structure and function. For example, in certain embodiments, the disclosed composition may be used to improve stuttering and other dyspraxias or to treat Parkinson's disease or schizophrenia.

The term “neurological disease or disorder” includes for example, Alzheimer's disease, mild cognitive impairment (MCI), Parkinson's disease, Parkinson's disease dementia, multiple sclerosis, adrenoleukodystrophy, AIDS dementia complex, Alexander disease, Alper's disease, amyotrophic lateral sclerosis (ALS), ataxia telangiectasia, Batten disease, bovine spongiform encephalopathy, Canavan disease, cerebral amyloid angiopathy, cerebellar ataxia, Cockayne syndrome, corticobasal degeneration, Creutzfeldt-Jakob disease, diffuse myelinoclastic sclerosis, fatal familial insomnia, Fazio-Londe disease, Friedreich's ataxia, frontotemporal dementia or lobar degeneration, hereditary spastic paraplegia, Huntington disease, Kennedy's disease, Krabbe disease, Lewy body dementia, Lyme disease, Machado-Joseph disease, motor neuron disease, Multiple systems atrophy, neuroacanthocytosis, Niemann-Pick disease, Pelizaeus-Merzbacher Disease, Pick's disease, primary lateral sclerosis including its juvenile form, progressive bulbar palsy, progressive supranuclear palsy, Refsum's disease including its infantile form, Sandhoff disease, Schilder's disease, spinal muscular atrophy, spinocerebellar ataxia, Steele-Richardson-Olszewski disease, subacute combined degeneration of the spinal cord, survival motor neuron spinal muscular atrophy, Tabes dorsalis, Tay-Sachs disease, toxic encephalopathy, transmissible spongiform encephalopathy, Vascular dementia, X-linked spinal muscular atrophy, synucleinopathy, progranulinopathy, tauopathy, amyloid disease, prion disease, protein aggregation disease, and movement disorder.

“Alkyl” is a branched, straight chain, or cyclic saturated hydrocarbon group including from 1 to about 8 carbon atoms, from 1 to about 6 carbon atoms, from 1 to about 4 carbon atoms, or from 1 to 3 carbon atoms. In certain embodiments, the alkyl is C₁-C₂, C₁-C₃, or C₁-C₄. The specified ranges as used herein indicate an alkyl group which is considered to explicitly disclose as individual species each member of the range described as a unique species. For example, the term C₁-C₄ alkyl as used herein indicates a straight or branched alkyl group having 1, 2, 3, or 4 carbon atoms and also a carbocyclic alkyl group of 3 or 4 carbon atoms and is intended to mean that each of these is described as an independent species. Examples of alkyl include, but are not limited to, methyl, ethyl, n-propyl, isopropyl, cyclopropyl, n-butyl, isobutyl, sec-butyl, cyclobutyl, t-butyl, n-pentyl, isopentyl, tert-pentyl, neopentyl, cyclopentyl, n-hexyl, cyclopentyl, 2-methylpentane, 3-methylpentane, 2,2-dimethylbutane, 2,3-dimethylbutane, and hexyl.

“Haloalkyl” indicates both branched and straight-chain alkyl groups substituted with one or more halogen atoms, up to the maximum allowable number of halogen atoms. Examples of haloalkyl include, but are not limited to, trifluoromethyl, monofluoromethyl, difluoromethyl, 2-fluoroethyl, 2,2,2-trifluoroethyl, and penta-fluoroethyl.

### Dosage Embodiments

In certain embodiments the formulation of the present invention includes an entactogen at a dose selected from the following embodiments:
a. MDMA ([1-(2H-1,3-benzodioxol-5-yl)propan-2-yl](methyl)amine), 20-400 mg
b. MDA (1-(2H-1,3-benzodioxol-5-yl)propan-2-amine), 20-300 mg
c. MDE ([1-(2H-1,3-benzodioxol-5-yl)propan-2-yl](ethyl)amine), 20-500 mg
d. MBDB ([1-(2H-1,3-benzodioxol-5-yl)butan-2-yl](methyl)amine), 40-400 mg
e. BDB (1-(2H-1,3-benzodioxol-5-yl)butan-2-amine), 40-400 mg
f. MDAI (2H,5H,6H,7H-indeno[5,6-d][1,3]dioxol-6-amine), 20-400 mg
g. MDMAI (N-methyl-2H,5H,6H,7H-indeno[5,6-d][1,3]dioxol-6-amine), 20-400 mg
h. MDAT (2H,5H,6H,7H,8H-naphtho[2,3-d][1,3]dioxol-6-amine), 20-600 mg
i. MDMAT (N-methyl-2H,5H,6H,7H,8H-naphtho[2,3-d][1,3]dioxol-6-amine), 20-600 mg
j. BK-MDMA (MDMC, Methylone, 1-(2H-1,3-benzodioxol-5-yl)-2-(methylamino)propan-1-one), 40-500 mg
k. BK-MDEA (MDEC, Ethylone, 1-(2H-1,3-benzodioxol-5-yl)-2-(ethylamino)propan-1-one), 40-500 mg
l. 5-APB (1-(1-benzofuran-5-yl)propan-2-amine), 10-200 mg
m. 6-APB (1-(1-benzofuran-6-yl)propan-2-amine), 10-200 mg
n. 5-MAPB ([1-(1-benzofuran-5-yl)propan-2-yl](methyl)amine), 10-200 mg
o. 6-MAPB ([1-(1-benzofuran-6-yl)propan-2-yl](methyl)amine), 10-200 mg
p. 5-MAPBT ([1-(1-benzothiophen-5-yl)propan-2-yl](methyl)amine), 10-400 mg
q. 6-MAPBT ([1-(1-benzothiophen-6-yl)propan-2-yl](methyl)amine), 10-400 mg
r. 5-EAPB ([1-(1-benzofuran-5-yl)propan-2-yl](ethyl)amine), 10-400 mg
s. 6-EAPB ([1-(1-benzofuran-6-yl)propan-2-yl](ethyl)amine), 10-400 mg
t. 5-MBPB ([1-(1-benzofuran-5-yl)butan-2-yl](methyl)amine), 10-200 mg
u. 6-MBPB ([1-(1-benzofuran-6-yl)butan-2-yl](methyl)amine), 10-200 mg
v. 5-MBPBT ([1-(1-benzothiophen-5-yl)butan-2-yl](methyl)amine), 10-400 mg
w. 6-MBPBT ([1-(1-benzothiophen-6-yl)butan-2-yl](methyl)amine), 10-400 mg
x. 5-EAPBT ([1-(1-benzothiophen-5-yl)propan-2-yl](ethyl)amine), 10-400 mg
y. 6-EAPBT ([1-(1-benzothiophen-6-yl)propan-2-yl](ethyl)amine), 10-400 mg
z. BK-5-MAPB (1-(1-benzofuran-5-yl)-2-(methylamino)propan-1-one), 5-200 mg
aa. BK-5-EAPB (1-(1-benzofuran-5-yl)-2-(ethylamino)propan-1-one), 5-200 mg

In other embodiments the formulation of the present invention includes an entactogen at a dose selected from the following embodiments:
ab. MDMA ([1-(2H-1,3-benzodioxol-5-yl)propan-2-yl](methyl)amine), 10-200 mg
ac. MDA (1-(2H-1,3-benzodioxol-5-yl)propan-2-amine), 10-150 mg
ad. MDE ([1-(2H-1,3-benzodioxol-5-yl)propan-2-yl](ethyl)amine), 10-250 mg
ae. MBDB ([1-(2H-1,3-benzodioxol-5-yl)butan-2-yl](methyl)amine), 20-200 mg
af. BDB (1-(2H-1,3-benzodioxol-5-yl)butan-2-amine), 20-200 mg
ag. MDAI (2H,5H,6H,7H-indeno[5,6-d][1,3]dioxol-6-amine), 10-200 mg
ah. MDMAI (N-methyl-2H,5H,6H,7H-indeno[5,6-d][1,3]dioxol-6-amine), 10-200 mg
ai. MDAT (2H,5H,6H,7H,8H-naphtho[2,3-d][1,3]dioxol-6-amine), 10-300 mg
aj. MDMAT (N-methyl-2H,5H,6H,7H,8H-naphtho[2,3-d][1,3]dioxol-6-amine), 10-300 mg
ak. BK-MDMA (MDMC, Methylone, 1-(2H-1,3-benzodioxol-5-yl)-2-(methylamino)propan-1-one), 20-250 mg
al. BK-MDEA (MDEC, Ethylone, 1-(2H-1,3-benzodioxol-5-yl)-2-(ethylamino)propan-1-one), 20-250 mg
am. 5-APB (1-(1-benzofuran-5-yl)propan-2-amine), 5-100 mg
an. 6-APB (1-(1-benzofuran-6-yl)propan-2-amine), 5-100 mg
ao. 5-MAPB ([1-(1-benzofuran-5-yl)propan-2-yl](methyl)amine), 5-100 mg
ap. 6-MAPB ([1-(1-benzofuran-6-yl)propan-2-yl](methyl)amine), 5-100 mg
aq. 5-MAPBT ([1-(1-benzothiophen-5-yl)propan-2-yl](methyl)amine), 5-200 mg
ar. 6-MAPBT ([1-(1-benzothiophen-6-yl)propan-2-yl](methyl)amine), 5-200 mg
as. 5-EAPB ([1-(1-benzofuran-5-yl)propan-2-yl](ethyl)amine), 5-200 mg
at. 6-EAPB ([1-(1-benzofuran-6-yl)propan-2-yl](ethyl)amine), 5-200 mg
au. 5-MBPB ([1-(1-benzofuran-5-yl)butan-2-yl](methyl)amine), 5-100 mg
av. 6-MBPB ([1-(1-benzofuran-6-yl)butan-2-yl](methyl)amine), 5-100 mg
aw. 5-MBPBT ([1-(1-benzothiophen-5-yl)butan-2-yl](methyl)amine), 5-200 mg
ax. 6-MBPBT ([1-(1-benzothiophen-6-yl)butan-2-yl](methyl)amine), 5-200 mg
ay. 5-EAPBT ([1-(1-benzothiophen-5-yl)propan-2-yl](ethyl)amine), 5-200 mg
az. 6-EAPBT ([1-(1-benzothiophen-6-yl)propan-2-yl](ethyl)amine), 5-200 mg
aaa. BK-5-MAPB (1-(1-benzofuran-5-yl)-2-(methylamino)propan-1-one), 2-100 mg
aab. BK-5-EAPB (1-(1-benzofuran-5-yl)-2-(ethylamino)propan-1-one), 2-100 mg

In certain embodiments the formulation of any one of embodiments a.-aab. further comprises an SSRI at a dose selected form the following embodiments:
i. Escitalopram, 5-30 mg
ii. Citalopram, 5-40 mg
iii. Fluoxetine, 10-80 mg
iv. Fluvoxamine, 20-300 mg
v. Paroxetine, 10-60 mg
vi. Sertraline, 20-300 mg
vii. Vortioxetine, 5-30 mg
viii. Vilazodone, 10-80 mg
ix. Femoxetine, 10-400 mg
x. Litoxetine, 5-300 mg
xi. McN 5652, 0.125-150 mg
xii. DASB (3-amino-4-[2-[(di(methyl)amino)methyl]phenyl]sulfanylbenzonitrile), 0.125-150 mg
xiii. Cyanodothepine, 0.125-150 mg
xiv. Cericlamine, 10-400 mg

In certain embodiments the formulation of any one of embodiments a.-aab. further comprises an SNRI at a dose selected form the following embodiments:
xv. Desvenlafaxine, 10-400 mg
xvi. Duloxetine, 10-120 mg
xvii. Levomilnacipran, 10-120 mg
xviii. Milnacipran, 5-300 mg
xix. Venlafaxine, 15-375 mg
xx. Tesofensine (NS 2330), 0.125-4 mg
xxi. DOV 216,303, 0.125-200 mg
xxii. DOV 21,947, 0.125-200 mg

In other embodiments the formulation of any one of embodiments a.-aab. further comprises an SSRI at a dose selected form the following embodiments:
xxiii. Escitalopram, 2-15 mg
xxiv. Citalopram, 2-20 mg
xxv. Fluoxetine, 5-40 mg
xxvi. Fluvoxamine, 10-150 mg
xxvii. Paroxetine, 5-30 mg
xxviii. Sertraline, 10-150 mg
xxix. Vortioxetine, 2-15 mg
xxx. Vilazodone, 5-40 mg
xxxi. Femoxetine, 5-200 mg
xxxii. Litoxetine, 2-150 mg
xxxiii. McN 5652, 0.06-75 mg
xxxiv. DASB (3-amino-4-[2-[(di(methyl)amino)methyl]phenyl]sulfanylbenzonitrile), 0.06-75 mg
xxxv. Cyanodothepine, 0.06-75 mg
xxxvi. Cericlamine, 5-200 mg

In certain other embodiments the formulation of any one of embodiments a.-aab. further comprises an SNRI at a dose selected form the following embodiments:
xxxvii. Desvenlafaxine, 5-200 mg
xxxviii. Duloxetine, 5-60 mg
xxxix. Levomilnacipran, 5-60 mg
xl. Milnacipran, 2-150 mg
xli. Venlafaxine, 5-200 mg
xlii. Tesofensine (NS 2330), 0.06-2 mg
xliii. DOV 216,303, 0.06-100 mg
xliv. DOV 21,947, 0.06-100 mg

This invention envisions the specific combination of each of the entactogens with each SSRI or SNRI combined independently as if each were separately written, as described further below.

### Additional Embodiments

1. A pharmaceutical composition that decreases the side effects of an entactogenic compound by providing the entactogenic compound as an immediate release component and second agent which is a serotonin reuptake inhibitor (SSRI) or serotonin and norepinephrine reuptake inhibitor (SNRI) as a delayed release component;
wherein:
the entactogenic compound is a compound selected from:
MDMA ([1-(2H-1,3-benzodioxol-5-yl)propan-2-yl](methyl)amine);
MDA (1-(2H-1,3-benzodioxol-5-yl)propan-2-amine);
MDE ([1-(2H-1,3-benzodioxol-5-yl)propan-2-yl](ethyl)amine);
MBDB ([1-(2H-1,3-benzodioxol-5-yl)butan-2-yl](methyl)amine);
BDB (1-(2H-1,3-benzodioxol-5-yl)butan-2-amine);
MDAI (2H,5H,6H,7H-indeno[5,6-d][1,3]dioxol-6-amine);
MDMAI (N-methyl-2H,5H,6H,7H-indeno[5,6-d][1,3]dioxol-6-amine);
MDAT (2H,5H,6H,7H,8H-naphtho[2,3-d][1,3]dioxol-6-amine);
MDMAT (N-methyl-2H,5H,6H,7H,8H-naphtho[2,3-d][1,3]dioxol-6-amine);
BK-MDMA (MDMC, Methylone, 1-(2H-1,3-benzodioxol-5-yl)-2-(methylamino)propan-1-one);
BK-MDEA (MDEC, Ethylone, 1-(2H-1,3-benzodioxol-5-yl)-2-(ethylamino)propan-1-one);
5-APB (1-(1-benzofuran-5-yl)propan-2-amine);
6-APB (1-(1-benzofuran-6-yl)propan-2-amine);
5-MAPB ([1-(1-benzofuran-5-yl)propan-2-yl](methyl)amine);
6-MAPB ([1-(1-benzofuran-6-yl)propan-2-yl](methyl)amine);
BK-5-MAPB (1-(1-benzofuran-5-yl)-2-(methylamino)propan-1-one);
BK-5-EAPB (1-(1-benzofuran-5-yl)-2-(ethylamino)propan-1-one);
5-MAPBT ([1-(1-benzothiophen-5-yl)propan-2-yl](methyl)amine);
6-MAPBT ([1-(1-benzothiophen-6-yl)propan-2-yl](methyl)amine);
5-EAPB ([1-(1-benzofuran-5-yl)propan-2-yl](ethyl)amine);
6-EAPB ([1-(1-benzofuran-6-yl)propan-2-yl](ethyl)amine);
5-MBPB ([1-(1-benzofuran-5-yl)butan-2-yl](methyl)amine);
6-MBPB ([1-(1-benzofuran-6-yl)butan-2-yl](methyl)amine);
5-MBPBT ([1-(1-benzothiophen-5-yl)butan-2-yl](methyl)amine);
6-MBPBT ([1-(1-benzothiophen-6-yl)butan-2-yl](methyl)amine);
5-EAPBT ([1-(1-benzothiophen-5-yl)propan-2-yl](ethyl)amine); and
6-EAPBT ([1-(1-benzothiophen-6-yl)propan-2-yl](ethyl)amine);
or a pharmaceutically acceptable salt thereof,
   the SSRI is a compound selected from citalopram, escitalopram, fluoxetine, fluvoxamine, paroxetine, sertraline, vortioxetine, and vilazodone; or a pharmaceutically acceptable salt thereof; and
   the SNRI is a compound selected from desvenlafaxine, duloxetine, levomilnacipran, milnacipran, and venlafaxine; or a pharmaceutically acceptable salt thereof.
   2. A pharmaceutical composition comprising:
   (1) an entactogen selected from:
MDMA ([1-(2H-1,3-benzodioxol-5-yl)propan-2-yl](methyl)amine);
MDA (1-(2H-1,3-benzodioxol-5-yl)propan-2-amine);
MDE ([1-(2H-1,3-benzodioxol-5-yl)propan-2-yl](ethyl)amine);
MBDB ([1-(2H-1,3-benzodioxol-5-yl)butan-2-yl](methyl)amine);
BDB (1-(2H-1,3-benzodioxol-5-yl)butan-2-amine);
MDAI (2H,5H,6H,7H-indeno[5,6-d][1,3]dioxol-6-amine);
MDMAI (N-methyl-2H,5H,6H,7H-indeno[5,6-d][1,3]dioxol-6-amine);
MDAT (2H,5H,6H,7H,8H-naphtho[2,3-d][1,3]dioxol-6-amine);
MDMAT (N-methyl-2H,5H,6H,7H,8H-naphtho[2,3-d][1,3]dioxol-6-amine);
BK-MDMA (MDMC, Methylone, 1-(2H-1,3-benzodioxol-5-yl)-2-(methylamino)propan-1-one);
BK-MDEA (MDEC, Ethylone, 1-(2H-1,3-benzodioxol-5-yl)-2-(ethylamino)propan-1-one);
5-APB (1-(1-benzofuran-5-yl)propan-2-amine);
6-APB (1-(1-benzofuran-6-yl)propan-2-amine);
5-MAPB ([1-(1-benzofuran-5-yl)propan-2-yl](methyl)amine);
BK-5-MAPB (1-(1-benzofuran-5-yl)-2-(methylamino)propan-1-one);
BK-5-EAPB (1-(1-benzofuran-5-yl)-2-(ethylamino)propan-1-one);
6-MAPB ([1-(1-benzofuran-6-yl)propan-2-yl](methyl)amine);
5-MAPBT ([1-(1-benzothiophen-5-yl)propan-2-yl](methyl)amine);
6-MAPBT ([1-(1-benzothiophen-6-yl)propan-2-yl](methyl)amine);
5-EAPB ([1-(1-benzofuran-5-yl)propan-2-yl](ethyl)amine);
6-EAPB ([1-(1-benzofuran-6-yl)propan-2-yl](ethyl)amine);
5-MBPB ([1-(1-benzofuran-5-yl)butan-2-yl](methyl)amine);
6-MBPB ([1-(1-benzofuran-6-yl)butan-2-yl](methyl)amine);
5-MBPBT ([1-(1-benzothiophen-5-yl)butan-2-yl](methyl)amine);
6-MBPBT ([1-(1-benzothiophen-6-yl)butan-2-yl](methyl)amine);
5-EAPBT ([1-(1-benzothiophen-5-yl)propan-2-yl](ethyl)amine); and
6-EAPBT ([1-(1-benzothiophen-6-yl)propan-2-yl](ethyl)amine);
or a pharmaceutically acceptable salt thereof;
   (2) a second agent, wherein the second agent is a serotonin reuptake inhibitor (SSRI) or serotonin and norepinephrine reuptake inhibitor (SNRI); wherein
   the SSRI is a compound selected from citalopram, escitalopram, fluoxetine, fluvoxamine, paroxetine, sertraline, vortioxetine, and vilazodone or a pharmaceutically acceptable salt thereof; and
   the SNRI is a compound selected from desvenlafaxine, duloxetine, levomilnacipran, milnacipran, and venlafaxine or a pharmaceutically acceptable salt thereof;
   (3) one or more additional pharmaceutically acceptable excipients;
      wherein:
   there is a delay between the maximum effects of the entactogen and the maximum effects of the SSRI or SNRI when administered to a human and wherein the pharmaceutical composition is an oral dosage form.
   3. The pharmaceutical composition of embodiment 1 or 2, wherein maximum concentration (Cmax) of SSRI or SNRI in the patient's blood is after the maximum concentration of entactogen in the patient's blood.
   4. The pharmaceutical composition of embodiment 3, wherein Cmax of SSRI or SNRI is about 1, 1.5, 2, 2.5, 3, 3.5, or 4 hours after the maximum concentration of the entactogen.
   5. The pharmaceutical composition of embodiment 3, wherein Cmax of SSRI or SNRI is about 1 hour after the maximum concentration of the entactogen.
   6. The pharmaceutical composition of embodiment 3, wherein Cmax of SSRI or SNRI is about 2 hours after the maximum concentration of the entactogen.
   7. The pharmaceutical composition of embodiment 3, wherein Cmax of SSRI or SNRI is about 3 hours after the maximum concentration of the entactogen.
   8. The pharmaceutical composition of embodiment 3, wherein Cmax of SSRI or SNRI is about 4 hours after the maximum concentration of the entactogen.
   9. The pharmaceutical composition of any one of embodiments 1-8, wherein the composition comprises 10-180 mg of MDMA or a pharmaceutically acceptable salt thereof.
   10. The pharmaceutical composition of any one of embodiments 1-8, wherein the composition comprises 5-90 mg of MDMA or a pharmaceutically acceptable salt thereof.
   11. The pharmaceutical composition of any one of embodiments 1-8, wherein the composition comprises 20-200 mg of MDA or a pharmaceutically acceptable salt thereof.
   12. The pharmaceutical composition of any one of embodiments 1-8, wherein the composition comprises 10-100 mg of MDA or a pharmaceutically acceptable salt thereof.
   13. The pharmaceutical composition of any one of embodiments 1-8, wherein the composition comprises 20-300 mg of MDE or a pharmaceutically acceptable salt thereof.
   14. The pharmaceutical composition of any one of embodiments 1-8, wherein the composition comprises 10-150 mg of MDE or a pharmaceutically acceptable salt thereof.
   15. The pharmaceutical composition of any one of embodiments 1-8, wherein the composition comprises 20-160 mg of 5-MAPB or a pharmaceutically acceptable salt thereof.
   16. The pharmaceutical composition of any one of embodiments 1-8, wherein the composition comprises 10-80 mg of 5-MAPB or a pharmaceutically acceptable salt thereof.
   17. The pharmaceutical composition of any one of embodiments 1-8, wherein the composition comprises 20-160 mg of 6-MAPB or a pharmaceutically acceptable salt thereof.
   18. The pharmaceutical composition of any one of embodiments 1-8, wherein the composition comprises 10-80 mg of 6-MAPB or a pharmaceutically acceptable salt thereof.
   19. The pharmaceutical composition of any one of embodiments 1-8, wherein the composition comprises 20-300 mg of MBDB or a pharmaceutically acceptable salt thereof.
   20. The pharmaceutical composition of any one of embodiments 1-8, wherein the composition comprises 10-150 mg of MBDB or a pharmaceutically acceptable salt thereof.
   21. The pharmaceutical composition of any one of embodiments 1-8, wherein the composition comprises 10-200 mg of 5-MBPB or a pharmaceutically acceptable salt thereof.
   22. The pharmaceutical composition of any one of embodiments 1-8, wherein the composition comprises 5-100 mg of 5-MBPB or a pharmaceutically acceptable salt thereof.
   23. The pharmaceutical composition of any one of embodiments 1-8, wherein the composition comprises 10-200 mg of 6-MBPB or a pharmaceutically acceptable salt thereof.
   24. The pharmaceutical composition of any one of embodiments 1-8, wherein the composition comprises 5-100 mg of 6-MBPB or a pharmaceutically acceptable salt thereof.
   25. The pharmaceutical composition of any one of embodiments 1-8, wherein the composition comprises 20-160 mg of 5-APB or a pharmaceutically acceptable salt thereof.
   26. The pharmaceutical composition of any one of embodiments 1-8, wherein the composition comprises 10-80 mg of 5-APB or a pharmaceutically acceptable salt thereof.
   27. The pharmaceutical composition of any one of embodiments 1-8, wherein the composition comprises 20-160 mg of 6-APB or a pharmaceutically acceptable salt thereof.
   28. The pharmaceutical composition of any one of embodiments 1-8, wherein the composition comprises 10-80 mg of 6-APB or a pharmaceutically acceptable salt thereof.
   29. A pharmaceutical composition that decreases the side effects of an entactogenic compound by providing the entactogenic compound as an immediate release component and a second agent which is a serotonin reuptake inhibitor (SSRI) or serotonin and norepinephrine reuptake inhibitor (SNRI) as a delayed release component;
   wherein:
   the entactogenic compound is a compound is of formula:

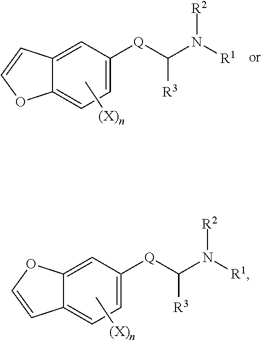

or a pharmaceutically acceptable salt or salt mixture thereof;
wherein:
R¹ and R² are independently selected from H, C₁-C₄ alkyl, C₁-C₄ haloalkyl, and —CH₂CH₂OH;
R³ is selected from H, C₁-C₄ alkyl, C₁-C₄ haloalkyl, CH₂OH, and —CH₂CH₂OH;
Q is

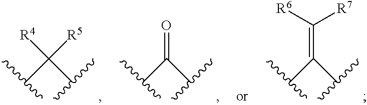

R⁴ and R⁵ are independently selected from H, C₁-C₄ alkyl, C₁-C₄ haloalkyl, F, Cl, and Br;
R⁶ and R⁷ are independently selected from H, C₁-C₄ alkyl, C₁-C₄ haloalkyl, F, Cl, and Br;
each X is independently selected from —F, —Cl, and —Br; and
n is 1 or 2;
the SSRI is a compound selected from citalopram, escitalopram, fluoxetine, fluvoxamine, paroxetine, sertraline, vortioxetine, and vilazodone; or a pharmaceutically acceptable salt thereof; and
the SNRI is a compound selected from desvenlafaxine, duloxetine, levomilnacipran, milnacipran, and venlafaxine; or a pharmaceutically acceptable salt thereof.
30. A pharmaceutical composition comprising:
(1) an entactogen of formula:

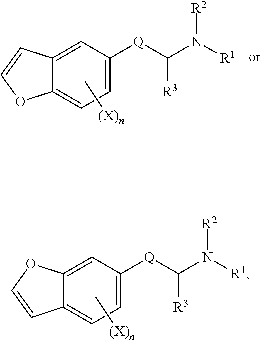

or a pharmaceutically acceptable salt or salt mixture thereof;
wherein:
R¹ and R² are independently selected from H, C₁-C₄ alkyl, C₁-C₄ haloalkyl, and —CH₂CH₂OH;
R³ is selected from H, C₁-C₄ alkyl, C₁-C₄ haloalkyl, CH₂OH, and —CH₂CH₂OH;
Q is

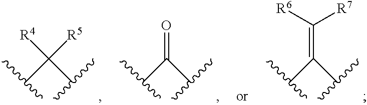

R⁴ and R⁵ are independently selected from H, C₁-C₄ alkyl, C₁-C₄ haloalkyl, F, Cl, and Br;
R⁶ and R⁷ are independently selected from H, C₁-C₄ alkyl, C₁-C₄ haloalkyl, F, Cl, and Br;
each X is independently selected from —F, —Cl, and —Br; and
n is 1 or 2;
(2) a second agent, wherein the second agent is a serotonin reuptake inhibitor (SSRI) or serotonin and norepinephrine reuptake inhibitor (SNRI); wherein
the SSRI is a compound selected from citalopram, escitalopram, fluoxetine, fluvoxamine, paroxetine, sertraline, vortioxetine, and vilazodone or a pharmaceutically acceptable salt thereof; and
the SNRI is a compound selected from desvenlafaxine, duloxetine, levomilnacipran, milnacipran, and venlafaxine or a pharmaceutically acceptable salt thereof; and
(3) one or more additional pharmaceutically acceptable excipients;
   wherein:
there is a delay between the maximum effects of the entactogen and the maximum effects of the SSRI or SNRI when administered to a human and wherein the pharmaceutical composition is an oral dosage form.
31. The pharmaceutical composition of embodiment 29 or 30, wherein the entactogen is selected from:

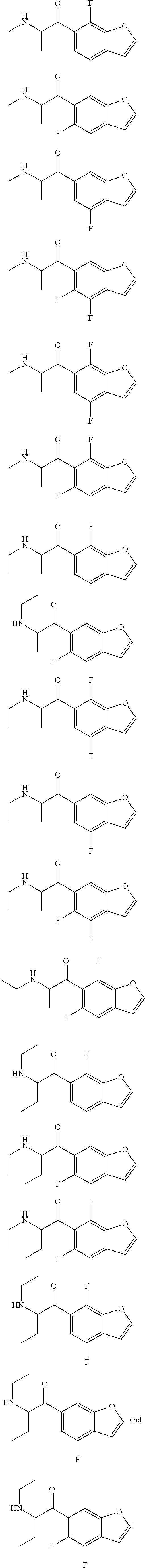

or a pharmaceutically acceptable salt thereof
32. The pharmaceutical composition of embodiment 29 or 30, wherein the entactogen is selected from:

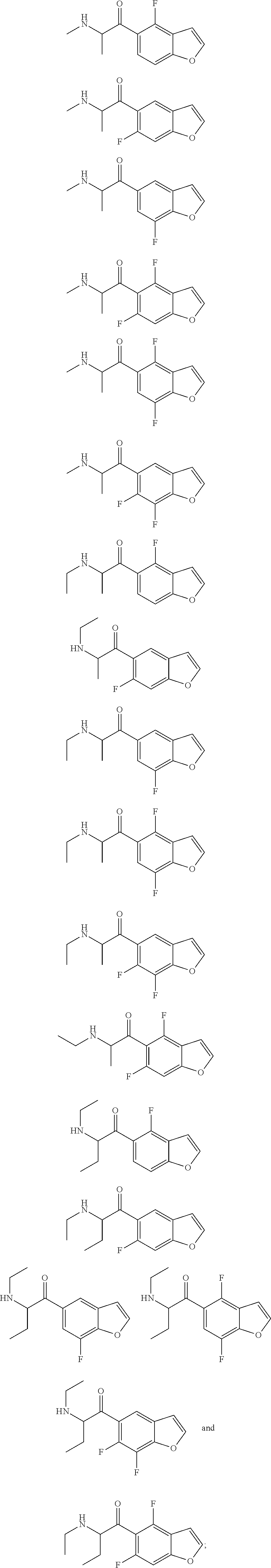

or a pharmaceutically acceptable salt thereof.
33. The pharmaceutical composition of embodiment 29 or 30, wherein the entactogen is selected from:

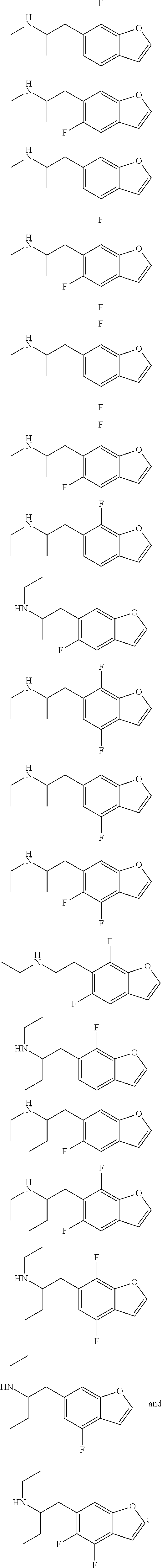

or a pharmaceutically acceptable salt thereof.
34. The pharmaceutical composition of embodiment 29 or 30, wherein the entactogen is selected from:

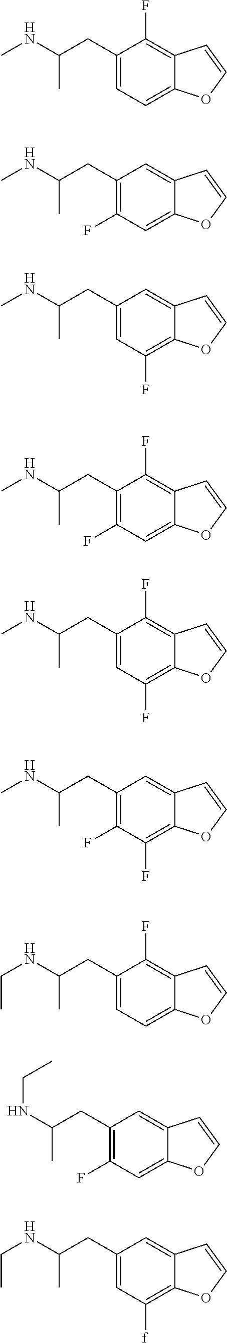

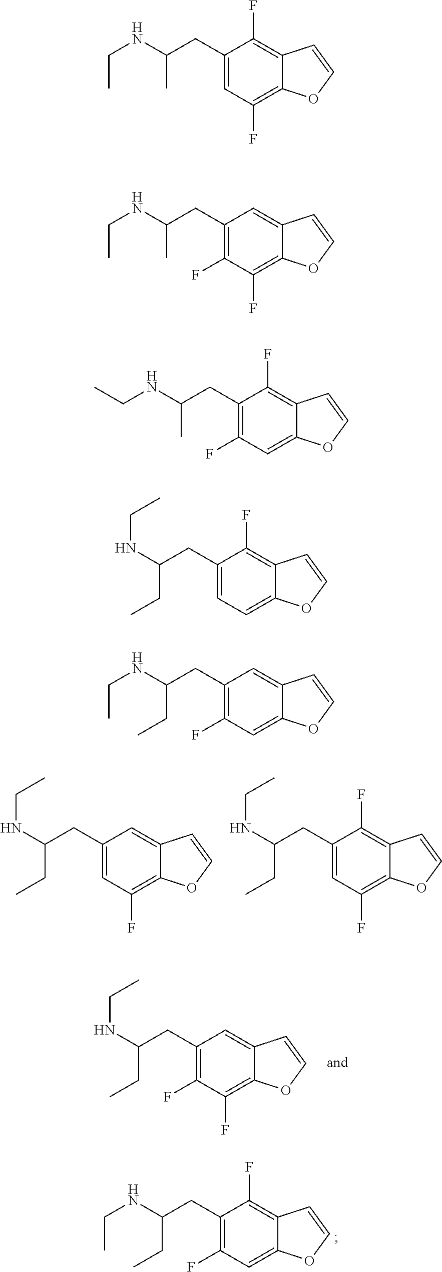

or a pharmaceutically acceptable salt thereof.
35. The pharmaceutical composition of any one of embodiments 1-34, wherein maximum concentration (Cmax) of SSRI or SNRI in the patient's blood is after the maximum concentration of entactogen in the patient's blood.
36. The pharmaceutical composition of embodiment 35, wherein Cmax of SSRI or SNRI is between 1 and 4 hours after the maximum concentration of the entactogen.
37. The pharmaceutical composition of embodiment 35, wherein Cmax of SSRI or SNRI is about 1 hour after the maximum concentration of the entactogen.
38. The pharmaceutical composition of embodiment 35, wherein Cmax of SSRI or SNRI is about 2 hours after the maximum concentration of the entactogen.
39. The pharmaceutical composition of embodiment 35, wherein Cmax of SSRI or SNRI is about 3 hours after the maximum concentration of the entactogen.
40. The pharmaceutical composition of embodiment 35, wherein Cmax of SSRI or SNRI is about 4 hours after the maximum concentration of the entactogen.
41. The pharmaceutical composition of any one of embodiments 1-40, wherein the entactogen is racemic.
42. The pharmaceutical composition of any one of embodiments 1-40, wherein the entactogen is enantiomerically enriched in the S-enantiomer.
43. The pharmaceutical composition of embodiment 42, wherein the entactogen is an enantiomerically enriched mixture with between 55% and 95% S-enantiomer.
44. The pharmaceutical composition of 42, wherein the entactogen is an enantiomerically enriched mixture with between 60% and 90% S-enantiomer.
45. The pharmaceutical composition of 42, wherein the entactogen is an enantiomerically enriched mixture with between 70% and 90% S-enantiomer.
46. The pharmaceutical composition of 42, wherein the entactogen is an enantiomerically enriched mixture with between 80% and 90% S-enantiomer.
47. The pharmaceutical composition of any one of embodiments 1-40, wherein the entactogen is enantiomerically enriched in the R-enantiomer.
48. The pharmaceutical composition of embodiment 47, wherein the entactogen is an enantiomerically enriched mixture with between 55% and 95% R-enantiomer.
49. The pharmaceutical composition of 47, wherein the entactogen is an enantiomerically enriched mixture with between 60% and 90% R-enantiomer.
50. The pharmaceutical composition of 47, wherein the entactogen is an enantiomerically enriched mixture with between 70% and 90% R-enantiomer.
51. The pharmaceutical composition of 47, wherein the entactogen is an enantiomerically enriched mixture with between 80% and 90% R-enantiomer.
52. The pharmaceutical composition of any one of embodiments 1-40, wherein the entactogen is the S-enantiomer.
53. The pharmaceutical composition of any one of embodiments 1-40, wherein the entactogen is the R-enantiomer.
54. The pharmaceutical composition of any one of embodiments 1-53, wherein the second agent is 10-400 mg of desvenlafaxine or a pharmaceutically acceptable salt thereof.
55. The pharmaceutical composition of any one of embodiments 1-53, wherein the second agent is 5-200 mg of desvenlafaxine or a pharmaceutically acceptable salt thereof.
56. The pharmaceutical composition of any one of embodiments 1-53, wherein the second agent is 10-120 mg of duloxetine or a pharmaceutically acceptable salt thereof.
57. The pharmaceutical composition of any one of embodiments 1-53, wherein the second agent is 5-60 mg of duloxetine or a pharmaceutically acceptable salt thereof.
58. The pharmaceutical composition of any one of embodiments 1-53, wherein the second agent is 10-120 mg of levomilnacipran or a pharmaceutically acceptable salt thereof.
59. The pharmaceutical composition of any one of embodiments 1-53, wherein the second agent is 5-60 mg of levomilnacipran or a pharmaceutically acceptable salt thereof.
60. The pharmaceutical composition of any one of embodiments 1-53, wherein the second agent is 5-300 mg of milnacipran or a pharmaceutically acceptable salt thereof.
61. The pharmaceutical composition of any one of embodiments 1-53, wherein the second agent is 2-150 mg of milnacipran or a pharmaceutically acceptable salt thereof.
62. The pharmaceutical composition of any one of embodiments 1-53, wherein the second agent is 15-375 mg of venlafaxine or a pharmaceutically acceptable salt thereof.
63. The pharmaceutical composition of any one of embodiments 1-53, wherein the second agent is 5-200 mg of venlafaxine or a pharmaceutically acceptable salt thereof.
64. The pharmaceutical composition of any one of embodiments 1-53, wherein the second agent is 5-40 mg of citalopram or a pharmaceutically acceptable salt thereof.
65. The pharmaceutical composition of any one of embodiments 1-53, wherein the second agent is 2-20 mg of citalopram or a pharmaceutically acceptable salt thereof.
66. The pharmaceutical composition of any one of embodiments 1-53, wherein the second agent is 5-30 mg of escitalopram or a pharmaceutically acceptable salt thereof.
67. The pharmaceutical composition of any one of embodiments 1-53, wherein the second agent is 2-15 mg of escitalopram or a pharmaceutically acceptable salt thereof.
68. The pharmaceutical composition of any one of embodiments 1-53, wherein the second agent is 10-80 mg of fluoxetine or a pharmaceutically acceptable salt thereof.
69. The pharmaceutical composition of any one of embodiments 1-53, wherein the second agent is 5-40 mg of fluoxetine or a pharmaceutically acceptable salt thereof.
70. The pharmaceutical composition of any one of embodiments 1-53, wherein the second agent is 20-300 mg of fluvoxamine or a pharmaceutically acceptable salt thereof.
71. The pharmaceutical composition of any one of embodiments 1-53, wherein the second agent is 10-150 mg of fluvoxamine or a pharmaceutically acceptable salt thereof.
72. The pharmaceutical composition of any one of embodiments 1-53, wherein the second agent is 10-60 mg of paroxetine or a pharmaceutically acceptable salt thereof.
73. The pharmaceutical composition of any one of embodiments 1-53, wherein the second agent is 5-30 mg of paroxetine or a pharmaceutically acceptable salt thereof.
74. The pharmaceutical composition of any one of embodiments 1-53, wherein the second agent is 20-300 mg of sertraline or a pharmaceutically acceptable salt thereof.
75. The pharmaceutical composition of any one of embodiments 1-53, wherein the second agent is 10-150 mg of sertraline or a pharmaceutically acceptable salt thereof.
76. The pharmaceutical composition of any one of embodiments 1-53, wherein the second agent is 5-30 mg of vortioxetine or a pharmaceutically acceptable salt thereof.
77. The pharmaceutical composition of any one of embodiments 1-53, wherein the second agent is 2-15 mg of vortioxetine or a pharmaceutically acceptable salt thereof.
78. The pharmaceutical composition of any one of embodiments 1-53, wherein the second agent is 10-80 mg of vilazodone or a pharmaceutically acceptable salt thereof.
79. The pharmaceutical composition of any one of embodiments 1-53, wherein the second agent is 5-40 mg of vilazodone or a pharmaceutically acceptable salt thereof.
80. The pharmaceutical composition of any one of embodiments 1-79, wherein the composition comprises delayed release granules of the second agent.
81. The pharmaceutical composition of any one of embodiments 1-79, wherein the composition comprises immediate release granules of the first agent.
82. The pharmaceutical composition of any one of embodiments 1-79, wherein the composition is a bilayer tablet.
83. The pharmaceutical composition of any one of embodiments 1-79, wherein the composition is a trilayer tablet.
84. A method for treating a central nervous system disorder comprising administering an effective amount of a pharmaceutical composition of anyone of embodiments 1-83 to a patient in need thereof.
85. The method of embodiment 84, wherein the method decreases an undesired effect selected from nausea, vomiting, anxiety, dizziness, sedation, drunkenness, difficulty concentrating, and mild confusion as compared to the administration of the entactogenic compound alone.
86. The method of embodiment 84 or 85, wherein the central nervous system disorder is selected from: post-traumatic stress disorder, depression, dysthymia, anxiety, generalized anxiety, social anxiety, panic, adjustment disorder, feeding and eating disorders, binge behaviors, body dysmorphic syndromes, addiction, drug abuse or dependence disorders, substance use disorders, disruptive behavior disorders, impulse control disorders, gaming disorders, gambling disorders, memory loss, dementia of aging, attention deficit hyperactivity disorder, personality disorders, attachment disorders, autism and dissociative disorders.
87. The method of any one of embodiments 84-85, wherein the central nervous system disorder is post-traumatic stress disorder.
88. The method of any one of embodiments 84-85, wherein the central nervous system disorder is adjustment disorder.
89. The method of any one of embodiments 84-85, wherein the central nervous system disorder is generalized anxiety.
90. The method of any one of embodiments 84-85, wherein the central nervous system disorder is social anxiety.
91. The method of any one of embodiments 84-85, wherein the central nervous system disorder is depression.
92. The method of any one of embodiments 84-85, wherein the central nervous system disorder is a substance use disorder.
93. The method of any one of embodiments 84-85, wherein the central nervous system disorder is an attachment disorder.
94. The method of any one of embodiments 84-85, wherein the central nervous system disorder is schizophrenia.
95. The method of any one of embodiments 84-85, wherein the central nervous system disorder is an eating disorder.
96. The method of embodiment 95 wherein the eating disorder is bulimia.
97. The method of embodiment 95 wherein the eating disorder is binge eating.
98. The method of embodiment 95 wherein the eating disorder is anorexia.
99. The method of any one of embodiments 84-98, wherein the pharmaceutical composition is administered in a clinical setting.
100. The method of any one of embodiments 84-98, wherein the pharmaceutical composition is administered in an at-home setting.
101. The method of any one of embodiments 84-98, wherein the pharmaceutical composition is administered during a psychotherapy session.
102. The method of any one of embodiments 84-98, wherein the pharmaceutical composition is administered during a counseling session.
103. A pharmaceutical composition of any one of embodiments 1-83 for treating a central nervous system disorder.
104. The pharmaceutical composition of embodiment 103, wherein the central nervous system disorder is selected from: post-traumatic stress disorder, depression, dysthymia, anxiety, generalized anxiety, social anxiety, panic, adjustment disorder, feeding and eating disorders, binge behaviors, body dysmorphic syndromes, addiction, drug abuse or dependence disorders, substance use disorders, disruptive behavior disorders, impulse control disorders, gaming disorders, gambling disorders, memory loss, dementia of aging, attention deficit hyperactivity disorder, personality disorders, attachment disorders, autism and dissociative disorders.
105. The pharmaceutical composition of embodiment 103, wherein the central nervous system disorder is post-traumatic stress disorder.
106. The pharmaceutical composition of embodiment 103, wherein the central nervous system disorder is adjustment disorder.
107. The pharmaceutical composition of embodiment 103, wherein the central nervous system disorder is generalized anxiety.
108. The pharmaceutical composition of embodiment 103, wherein the central nervous system disorder is social anxiety.
109. The pharmaceutical composition of embodiment 103, wherein the central nervous system disorder is depression.
110. The pharmaceutical composition of embodiment 103, wherein the central nervous system disorder is a substance use disorder.
111. The pharmaceutical composition of embodiment 103, wherein the central nervous system disorder is an attachment disorder.
112. The pharmaceutical composition of embodiment 103, wherein the central nervous system disorder is schizophrenia.
113. The pharmaceutical composition of embodiment 103, wherein the central nervous system disorder is an eating disorder.
114. The pharmaceutical composition of embodiment 113, wherein the eating disorder is bulimia.
115. The pharmaceutical composition of embodiment 113, wherein the eating disorder is binge eating.
116. The pharmaceutical composition of embodiment 113, wherein the eating disorder is anorexia.
117. Use of a pharmaceutical composition of any one of embodiments 1-83 for treating a central nervous system disorder.
118. Use of a pharmaceutical composition of any one of embodiments 1-83 in the manufacture of a medicament for treating a central nervous system disorder.
119. The use of embodiment 117 or 118, wherein an undesired effect selected from nausea, vomiting, anxiety, dizziness, sedation, drunkenness, difficulty concentrating, and mild confusion is decreased as compared to the administration of the entactogenic compound alone.
120. The use of embodiment 117 or 118, wherein the central nervous system disorder is selected from: post-traumatic stress disorder, depression, dysthymia, anxiety, generalized anxiety, social anxiety, panic, adjustment disorder, feeding and eating disorders, binge behaviors, body dysmorphic syndromes, addiction, drug abuse or dependence disorders, substance use disorders, disruptive behavior disorders, impulse control disorders, gaming disorders, gambling disorders, memory loss, dementia of aging, attention deficit hyperactivity disorder, personality disorders, attachment disorders, autism and dissociative disorders.
121. The use of embodiment 117 or 118, wherein the central nervous system disorder is post-traumatic stress disorder.
122. The use of embodiment 117 or 118, wherein the central nervous system disorder is adjustment disorder.
123. The use of embodiment 117 or 118, wherein the central nervous system disorder is generalized anxiety.
124. The use of embodiment 117 or 118, wherein the central nervous system disorder is social anxiety.
125. The use of embodiment 117 or 118, wherein the central nervous system disorder is depression.
126. The use of embodiment 117 or 118, wherein the central nervous system disorder is a substance use disorder.
127. The use of embodiment 117 or 118, wherein the central nervous system disorder is an attachment disorder.
128. The use of embodiment 117 or 118, wherein the central nervous system disorder is schizophrenia.
129. The use of embodiment 117 or 118, wherein the central nervous system disorder is an eating disorder.
130. The use of embodiment 129 wherein the eating disorder is bulimia.
131. The use of embodiment 129 wherein the eating disorder is binge eating.
132. The use of embodiment 129 wherein the eating disorder is anorexia.

### Methods of the Present Invention

The current invention discloses unexpected benefits from specific methods of coadministration of an entactogen and a SSRI or SNRI.

In certain embodiments kinetic lags that are contemplated include 0.5 hour, 1 hour, 1.5 hours, 2 hours, 3 hours, 4 hours, 5, hours, 6 hours, 7 hours, or 8 hours (for example, but not limited to between 0.5 and 8 hours, between 1 and 8 hours, between 1 and 7 hours, 2 and 6 hours, 2 and 5 hours, or 2 and 4 hours).

In certain embodiments Tmax of at least one of the active agents is measured by in vivo methods, wherein Tmax generally refers to the time at which the maximum concentration of the drug is detected in plasma.

In certain other embodiments, wherein the SSRI or SNRI exhibits slower absorption (Tmax>3 hours) from the digestive tract to general circulation, Tmax is measured with USP apparatus.

In other embodiments, an entactogen and a SSRI or SNRI are co-administered so that the entactogen reaches at least 50%, 60%, 70%, 80%, 90%, or at least 100% of its plasma Cmax before the SSRI or SNRI.

The present invention provides methods and uses for the treatment of CNS disorders, including, but not limited to, mental disorders as described herein, including post-traumatic stress and adjustment disorders, comprising administering a combination of an entactogenic compound or a pharmaceutically acceptable salt or mixture of salts thereof and a SSRI or SNRI, or a pharmaceutically acceptable salt or mixture of salts thereof as described herein. These combinations display many pharmacological properties that are beneficial to their use as therapeutics and represent an improvement over existing therapeutics.

In addition to treating various diseases and disorders, the employed methods of modulating activity of the serotonergic system in particular can be used to improve CNS functioning in non-disease states, such as reducing neuroticism and psychological defensiveness, increasing openness to experience, increasing creativity, and aiding decision-making.

The following nonlimiting examples are relevant to any of the disorders, indications, methods of use or dosing regimens described herein. When a host is treated with an effective amount of an enantiomerically enriched mixture of enantiomers of a compound described herein that treatment can either be in the form of one enantiomer being the immediate release agent and the other being the delayed release agent or both enantiomers can be dosed together as an immediate release agent and followed by another agent described herein after a kinetic lag. In certain embodiments a host is treated with an effective amount of enantiomerically enriched mixture of entactogen in combination with a racemic or pure enantiomer of a SSRI or SNRI.

In certain aspects the invention provides a method described below:
1. A method of improving the therapeutic profile of an entactogenic agent, involving co-administration of a SSRI or SNRI formulated for a delayed release, that decrease negative side effects associated with entactogen administration.
2. The method of 1 where the kinetic lag is a difference in Tmax of the entactogen and SSRI or SNRI is about or between 0.5, 1, 1.5, 2, 3, 4, 5, 6, 7, or 8 hours (for example, but not limited to between 0.5 and 8 hours, between 1 and 8 hours, between 1 and 7 hours, 2 and 6 hours, 2 and 5 hours, or 2 and 4 hours).
3. The method of 1 where the kinetic lag is a difference in timing of the 50% Cmax of the entactogen and SSRI or SNRI is about or between 0.5, 1, 1.5, 2, 3, 4, 5, 6, 7, or 8 hours (for example, but not limited to between 0.5 and 8 hours, between 1 and 8 hours, between 1 and 7 hours, 2 and 6 hours, 2 and 5 hours, or 2 and 4 hours), as well as values in between these numbers.
4. The method of 1-3 where the entactogen and SSRI or SNRI are in the same dosage form and have different release profiles.
5. The method of 4 where the dosage forms are oral.
6. The method of 1-5 where the entactogen is MDMA and the SSRI is selected from citalopram, escitalopram, sertraline, fluoxetine, paroxetine, fluvoxamine, and vilazodone.
7. The method of 1-5 where the entactogen is MDMA and the SNRI is selected from duloxetine, milnacipran, levomilnacipran, venlafaxine, and desvenlafaxine.
8. The method of 1-5 where the entactogen is MDA and the SSRI is selected from citalopram, escitalopram, sertraline, fluoxetine, paroxetine, fluvoxamine, and vilazodone.
9. The method of 1-5 where the entactogen is MDA and the SNRI is selected from duloxetine, milnacipran, levomilnacipran, venlafaxine, and desvenlafaxine.
10. The method of 1-5 where the entactogen is MDE and the SSRI is selected from citalopram, escitalopram, sertraline, fluoxetine, paroxetine, fluvoxamine, and vilazodone.
11. The method of 1-5 where the entactogen is MDE and the SNRI is selected from duloxetine, milnacipran, levomilnacipran, venlafaxine, and desvenlafaxine.
12. The method of 1-5 where the entactogen is MBDB and the SSRI is selected from citalopram, escitalopram, sertraline, fluoxetine, paroxetine, fluvoxamine, and vilazodone.
13. The method of 1-5 where the entactogen is MBDB and the SNRI is selected from duloxetine, milnacipran, levomilnacipran, venlafaxine, and desvenlafaxine.
14. The method of 1-5 where the entactogen is MDAI and the SSRI is selected from citalopram, escitalopram, sertraline, fluoxetine, paroxetine, fluvoxamine, and vilazodone.
15. The method of 1-5 where the entactogen is MDAI and the SNRI is selected from duloxetine, milnacipran, levomilnacipran, venlafaxine, and desvenlafaxine.
16. The method of 1-5 where the entactogen is MDMAI and the SSRI is selected from citalopram, escitalopram, sertraline, fluoxetine, paroxetine, fluvoxamine, and vilazodone.
17. The method of 1-5 where the entactogen is MDMAI and the SNRI is selected from duloxetine, milnacipran, levomilnacipran, venlafaxine, and desvenlafaxine.
18. The method of 1-5 where the entactogen is MDAT and the SSRI is selected from citalopram, escitalopram, sertraline, fluoxetine, paroxetine, fluvoxamine, and vilazodone.
19. The method of 1-5 where the entactogen is MDAT and the SNRI is selected from duloxetine, milnacipran, levomilnacipran, venlafaxine, and desvenlafaxine.
20. The method of 1-5 where the entactogen is MDMAT and the SSRI is selected from citalopram, escitalopram, sertraline, fluoxetine, paroxetine, fluvoxamine, and vilazodone.
21. The method of 1-5 where the entactogen is MDMAT and the SNRI is selected from duloxetine, milnacipran, levomilnacipran, venlafaxine, and desvenlafaxine.
22. The method of 1-5 where the entactogen is BK-MDMA and the SSRI is selected from citalopram, escitalopram, sertraline, fluoxetine, paroxetine, fluvoxamine, and vilazodone.
23. The method of 1-5 where the entactogen is BK-MDMA and SNRI is selected from duloxetine, milnacipran, levomilnacipran, venlafaxine, and desvenlafaxine.
24. The method of 1-5 where the entactogen is BK-MDEA and the SSRI is selected from citalopram, escitalopram, sertraline, fluoxetine, paroxetine, fluvoxamine, and vilazodone.
25. The method of 1-5 where the entactogen is BK-MDEA and the SNRI is selected from duloxetine, milnacipran, levomilnacipran, venlafaxine, and desvenlafaxine.
26. The method of 1-5 where the entactogen is 5-APB and the SSRI is selected from citalopram, escitalopram, sertraline, fluoxetine, paroxetine, fluvoxamine, and vilazodone.
27. The method of 1-5 where the entactogen is 5-APB and the SNRI is selected from duloxetine, milnacipran, levomilnacipran, venlafaxine, and desvenlafaxine.
28. The method of 1-5 where the entactogen is 6-APB and the SSRI is selected from citalopram, escitalopram, sertraline, fluoxetine, paroxetine, fluvoxamine, and vilazodone.
29. The method of 1-5 where the entactogen is 6-APB and the SNRI is selected from duloxetine, milnacipran, levomilnacipran, venlafaxine, and desvenlafaxine.
30. The method of 1-5 where the entactogen is 5-MAPB and the SSRI is selected from citalopram, escitalopram, sertraline, fluoxetine, paroxetine, fluvoxamine, and vilazodone.
31. The method of 1-5 where the entactogen is 5-MAPB and the SNRI is selected from duloxetine, milnacipran, levomilnacipran, venlafaxine, and desvenlafaxine.
32. The method of 1-5 where the entactogen is 6-MAPB and the SSRI is selected from citalopram, escitalopram, sertraline, fluoxetine, paroxetine, fluvoxamine, and vilazodone.
33. The method of 1-5 where the entactogen is 6-MAPB and the SNRI is selected from duloxetine, milnacipran, levomilnacipran, venlafaxine, and desvenlafaxine.
34. The method of 8-33 where the entactogen is enantiomerically enriched.
35. The method of 1-34 where the decreased negative effects are any of nausea, vomiting, anxiety, dizziness, sedation, drunkenness, difficulty concentrating, and mild confusion.
36. The method of 1-34 where the desired effects are decreased neuroticism, increased authenticity, and positive mood.
37. The method of 1-36 wherein the formulation is used for a one-dose psychotherapy.
38. The method of 1-36 wherein the formulation is used for a two-dose psychotherapy.

This invention also provides the administration of an effective amount of an entactogen, for example MDMA and an SSRI or a pharmaceutically acceptable salt or composition to a host, typically a human, to treat a maladaptive response to perceived psychological threats. In one embodiment, MDMA and the SSRI or a pharmaceutically acceptable salt or composition is administered in the context of psychotherapy. In one embodiment, MDMA and the SSRI or a pharmaceutically acceptable salt or composition is administered as a stand-alone treatment.

This invention also provides the administration of an effective amount of an entactogen, for example MDMA and the SNRI or a pharmaceutically acceptable salt or composition to a host, typically a human, to treat a maladaptive response to perceived psychological threats. In one embodiment, MDMA and the SNRI or a pharmaceutically acceptable salt or composition is administered in the context of psychotherapy. In one embodiment, MDMA and the SNRI or a pharmaceutically acceptable salt or composition is administered as a stand-alone treatment.

In certain embodiments, a host, for example a human, is treated with an effective amount of a formulation comprising an SSRI or SNRI and an enantiomerically enriched mixture of an entactogen described herein, or a pharmaceutically acceptable salt or mixed salt, isotopic derivative, or prodrug thereof, wherein the percent of S enantiomer is greater than about 99 percent.

In certain embodiments, a host, for example a human, is treated with an effective amount of a formulation comprising an SSRI or SNRI and an enantiomerically enriched mixture of an entactogen described herein, or a pharmaceutically acceptable salt or mixed salt, isotopic derivative, or prodrug thereof, wherein the percent of S enantiomer is greater than about 95 percent.

In certain embodiments, a host, for example a human, is treated with an effective amount of a formulation comprising an SSRI or SNRI and an enantiomerically enriched mixture of an entactogen described herein, or a pharmaceutically acceptable salt or mixed salt, isotopic derivative, or prodrug thereof, wherein the percent of S enantiomer is greater than about 90 percent.

In certain embodiments, a host, for example a human, is treated with an effective amount of a formulation comprising an SSRI or SNRI and an enantiomerically enriched mixture of an entactogen described herein, or a pharmaceutically acceptable salt or mixed salt, isotopic derivative, or prodrug thereof, wherein the percent of S enantiomer is greater than about 85 percent.

In certain embodiments, a host, for example a human, is treated with an effective amount of a formulation comprising an SSRI or SNRI and an enantiomerically enriched mixture of an entactogen described herein, or a pharmaceutically acceptable salt or mixed salt, isotopic derivative, or prodrug thereof, wherein the percent of S enantiomer is greater than about 80 percent.

In certain embodiments, a host, for example a human, is treated with an effective amount of a formulation comprising an SSRI or SNRI and an enantiomerically enriched mixture of an entactogen described herein, or a pharmaceutically acceptable salt or mixed salt, isotopic derivative, or prodrug thereof, wherein the percent of S enantiomer is greater than about 75 percent.

In certain embodiments, a host, for example a human, is treated with an effective amount of a formulation comprising an SSRI or SNRI and an enantiomerically enriched mixture of an entactogen described herein, or a pharmaceutically acceptable salt or mixed salt, isotopic derivative, or prodrug thereof, wherein the percent of S enantiomer is greater than about 70 percent.

In certain embodiments, a host, for example a human, is treated with an effective amount of a formulation comprising an SSRI or SNRI and an enantiomerically enriched mixture of an entactogen described herein, or a pharmaceutically acceptable salt or mixed salt, isotopic derivative, or prodrug thereof, wherein the percent of S enantiomer is greater than about 65 percent.

In certain embodiments, a host, for example a human, is treated with an effective amount of a formulation comprising an SSRI or SNRI and an enantiomerically enriched mixture of an entactogen described herein, or a pharmaceutically acceptable salt or mixed salt, isotopic derivative, or prodrug thereof, wherein the percent of S enantiomer is greater than about 60 percent.

In certain embodiments, a host, for example a human, is treated with an effective amount of a formulation comprising an SSRI or SNRI and an enantiomerically enriched mixture of an entactogen described herein, or a pharmaceutically acceptable salt or mixed salt, isotopic derivative, or prodrug thereof, wherein the percent of S enantiomer is greater than about 55 percent.

### Exemplary Formulations for Use in the Present Invention

i. Entactogen formulated for immediate release: 20-400 mg of pure or enantiomerically enriched R-MDMA, S-MDMA, or racemic MDMA, and a SSRI formulated for delayed release: 5-40 mg of citalopram;
ii. Entactogen formulated for immediate release: 20-400 mg of pure or enantiomerically enriched R-MDMA, S-MDMA, or racemic MDMA, and a SSRI formulated for delayed release: 5-30 mg of escitalopram;
iii. Entactogen formulated for immediate release: 20-400 mg of pure or enantiomerically enriched R-MDMA, S-MDMA, or racemic MDMA, and a SSRI formulated for delayed release: 10-80 mg of fluoxetine;
iv. Entactogen formulated for immediate release: 20-400 mg of pure or enantiomerically enriched R-MDMA, S-MDMA, or racemic MDMA, and a SSRI formulated for delayed release: 10-60 mg of paroxetine;
v. Entactogen formulated for immediate release: 20-400 mg of pure or enantiomerically enriched R-MDMA, S-MDMA, or racemic MDMA, and a SSRI formulated for delayed release: 20-300 mg of sertraline;
vi. Entactogen formulated for immediate release: 20-400 mg of pure or enantiomerically enriched R-MDMA, S-MDMA, or racemic MDMA, and a SSRI formulated for delayed release: 20-300 mg of fluvoxamine;
vii. Entactogen formulated for immediate release: 20-400 mg of pure or enantiomerically enriched R-MDMA, S-MDMA, or racemic MDMA, and a SSRI formulated for delayed release: 10-80 mg of vilazodone;
viii. Entactogen formulated for immediate release: 20-400 mg of pure or enantiomerically enriched R-MDMA, S-MDMA, or racemic MDMA, and a SSRI formulated for delayed release: 5-30 mg of vortioxetine;
ix. Entactogen formulated for immediate release: 20-400 mg of pure or enantiomerically enriched R-MDMA, S-MDMA, or racemic MDMA, and a SNRI formulated for delayed release: 10-120 mg of duloxetine;
x. Entactogen formulated for immediate release: 20-400 mg of pure or enantiomerically enriched R-MDMA, S-MDMA, or racemic MDMA, and a SNRI formulated for delayed release: 5-300 mg of milnacipran;
xi. Entactogen formulated for immediate release: 20-400 mg of pure or enantiomerically enriched R-MDMA, S-MDMA, or racemic MDMA, and a SNRI formulated for delayed release: 10-120 mg of levomilnacipran;
xii. Entactogen formulated for immediate release: 20-400 mg of pure or enantiomerically enriched R-MDMA, S-MDMA, or racemic MDMA, and a SNRI formulated for delayed release: 15-375 mg of venlafaxine;
xiii. Entactogen formulated for immediate release: 20-400 mg of pure or enantiomerically enriched R-MDMA, S-MDMA, or racemic MDMA, and a SNRI formulated for delayed release: 10-400 mg of desvenlafaxine;
xiv. Entactogen formulated for immediate release: 20-300 mg of pure or enantiomerically enriched R-MDA, S-MDA, or racemic MDA, and a SSRI formulated for delayed release: 5-40 mg of citalopram;
xv. Entactogen formulated for immediate release: 20-300 mg of pure or enantiomerically enriched R-MDA, S-MDA, or racemic MDA, and a SSRI formulated for delayed release: 5-30 mg of escitalopram;
xvi. Entactogen formulated for immediate release: 20-300 mg of pure or enantiomerically enriched R-MDA, S-MDA, or racemic MDA, and a SSRI formulated for delayed release: 10-80 mg of fluoxetine;
xvii. Entactogen formulated for immediate release: 20-300 mg of pure or enantiomerically enriched R-MDA, S-MDA, or racemic MDA, and a SSRI formulated for delayed release: 10-60 mg of paroxetine;
xviii. Entactogen formulated for immediate release: 20-300 mg of pure or enantiomerically enriched R-MDA, S-MDA, or racemic MDA, and a SSRI formulated for delayed release: 20-300 mg of sertraline;
xix. Entactogen formulated for immediate release: 20-300 mg of pure or enantiomerically enriched R-MDA, S-MDA, or racemic MDA, and a SSRI formulated for delayed release: 20-300 mg of fluvoxamine;
xx. Entactogen formulated for immediate release: 20-300 mg of pure or enantiomerically enriched R-MDA, S-MDA, or racemic MDA, and a SSRI formulated for delayed release: 10-80 mg of vilazodone;
xxi. Entactogen formulated for immediate release: 20-300 mg of pure or enantiomerically enriched R-MDA, S-MDA, or racemic MDA, and a SSRI formulated for delayed release: 5-30 mg of vortioxetine;
xxii. Entactogen formulated for immediate release: 20-300 mg of pure or enantiomerically enriched R-MDA, S-MDA, or racemic MDA, and a SNRI formulated for delayed release: 10-120 mg of duloxetine; and
xxiii. Entactogen formulated for immediate release: 20-300 mg of pure or enantiomerically enriched R-MDA, S-MDA, or racemic MDA, and a SNRI formulated for delayed release: 5-300 mg of milnacipran.
xxiv. Entactogen formulated for immediate release: 20-300 mg of pure or enantiomerically enriched R-MDA, S-MDA, or racemic MDA, and a SNRI formulated for delayed release: 10-120 mg of levomilnacipran;
xxv. Entactogen formulated for immediate release: 20-300 mg of pure or enantiomerically enriched R-MDA, S-MDA, or racemic MDA, and a SNRI formulated for delayed release: 15-375 mg of venlafaxine;
xxvi. Entactogen formulated for immediate release: 20-300 mg of pure or enantiomerically enriched R-MDA, S-MDA, or racemic MDA, and a SNRI formulated for delayed release: 10-400 mg of desvenlafaxine;
xxvii. Entactogen formulated for immediate release: 20-500 mg of pure or enantiomerically enriched R-MDE, S-MDE, or racemic MDE, and a SSRI formulated for delayed release: 5-40 mg of citalopram;
xxviii. Entactogen formulated for immediate release: 20-500 mg of pure or enantiomerically enriched R-MDE, S-MDE, or racemic MDE, and a SSRI formulated for delayed release: 5-30 mg of escitalopram;
xxix. Entactogen formulated for immediate release: 20-500 mg of pure or enantiomerically enriched R-MDE, S-MDE, or racemic MDE, and a SSRI formulated for delayed release: 10-80 mg of fluoxetine;
xxx. Entactogen formulated for immediate release: 20-500 mg of pure or enantiomerically enriched R-MDE, S-MDE, or racemic MDE, and a SSRI formulated for delayed release: 10-60 mg of paroxetine;
xxxi. Entactogen formulated for immediate release: 20-500 mg of pure or enantiomerically enriched R-MDE, S-MDE, or racemic MDE, and a SSRI formulated for delayed release: 20-300 mg of sertraline;
xxxii. Entactogen formulated for immediate release: 20-500 mg of pure or enantiomerically enriched R-MDE, S-MDE, or racemic MDE, and a SSRI formulated for delayed release: 20-300 mg of fluvoxamine;
xxxiii. Entactogen formulated for immediate release: 20-500 mg of pure or enantiomerically enriched R-MDE, S-MDE, or racemic MDE, and a SSRI formulated for delayed release: 10-80 mg of vilazodone;
xxxiv. Entactogen formulated for immediate release: 20-500 mg of pure or enantiomerically enriched R-MDE, S-MDE, or racemic MDE, and a SSRI formulated for delayed release: 5-30 mg of vortioxetine;
xxxv. Entactogen formulated for immediate release: 20-500 mg of pure or enantiomerically enriched R-MDE, S-MDE, or racemic MDE, and a SNRI formulated for delayed release: 10-120 mg of duloxetine; and
xxxvi. Entactogen formulated for immediate release: 20-500 mg of pure or enantiomerically enriched R-MDE, S-MDE, or racemic MDE, and a SNRI formulated for delayed release: 5-300 mg of milnacipran.
xxxvii. Entactogen formulated for immediate release: 20-500 mg of pure or enantiomerically enriched R-MDE, S-MDE, or racemic MDE, and a SNRI formulated for delayed release: 10-120 mg of levomilnacipran;
xxxviii. Entactogen formulated for immediate release: 20-500 mg of pure or enantiomerically enriched R-MDE, S-MDE, or racemic MDE, and a SNRI formulated for delayed release: 15-375 mg of venlafaxine;
xxxix. Entactogen formulated for immediate release: 20-500 mg of pure or enantiomerically enriched R-MDE, S-MDE, or racemic MDE, and a SNRI formulated for delayed release: 10-400 mg of desvenlafaxine;
xl. Entactogen formulated for immediate release: 40-400 mg of pure or enantiomerically enriched R-MBDB, S-MBDB, or racemic MBDB, and a SSRI formulated for delayed release: 5-40 mg of citalopram;
xli. Entactogen formulated for immediate release: 40-400 mg of pure or enantiomerically enriched R-MBDB, S-MBDB, or racemic MBDB, and a SSRI formulated for delayed release: 5-30 mg of escitalopram;
xlii. Entactogen formulated for immediate release: 40-400 mg of pure or enantiomerically enriched R-MBDB, S-MBDB, or racemic MBDB, and a SSRI formulated for delayed release: 10-80 mg of fluoxetine;
xliii. Entactogen formulated for immediate release: 40-400 mg of pure or enantiomerically enriched R-MBDB, S-MBDB, or racemic MBDB, and a SSRI formulated for delayed release: 10-60 mg of paroxetine;
xliv. Entactogen formulated for immediate release: 40-400 mg of pure or enantiomerically enriched R-MBDB, S-MBDB, or racemic MBDB, and a SSRI formulated for delayed release: 20-300 mg of sertraline;
xlv. Entactogen formulated for immediate release: 40-400 mg of pure or enantiomerically enriched R-MBDB, S-MBDB, or racemic MBDB, and a SSRI formulated for delayed release: 20-300 mg of fluvoxamine;
xlvi. Entactogen formulated for immediate release: 40-400 mg of pure or enantiomerically enriched R-MBDB, S-MBDB, or racemic MBDB, and a SSRI formulated for delayed release: 10-80 mg of vilazodone;
xlvii. Entactogen formulated for immediate release: 40-400 mg of pure or enantiomerically enriched R-MBDB, S-MBDB, or racemic MBDB, and a SSRI formulated for delayed release: 5-30 mg of vortioxetine;
xlviii. Entactogen formulated for immediate release: 40-400 mg of pure or enantiomerically enriched R-MBDB, S-MBDB, or racemic MBDB, and a SNRI formulated for delayed release: 10-120 mg of duloxetine; and
xlix. Entactogen formulated for immediate release: 40-400 mg of pure or enantiomerically enriched R-MBDB, S-MBDB, or racemic MBDB, and a SNRI formulated for delayed release: 5-300 mg of milnacipran.
l. Entactogen formulated for immediate release: 40-400 mg of pure or enantiomerically enriched R-MBDB, S-MBDB, or racemic MBDB, and a SNRI formulated for delayed release: 10-120 mg of levomilnacipran;
li. Entactogen formulated for immediate release: 40-400 mg of pure or enantiomerically enriched R-MBDB, S-MBDB, or racemic MBDB, and a SNRI formulated for delayed release: 15-375 mg of venlafaxine;
lii. Entactogen formulated for immediate release: 40-400 mg of pure or enantiomerically enriched R-MBDB, S-MBDB, or racemic MBDB, and a SNRI formulated for delayed release: 10-400 mg of desvenlafaxine;
liii. Entactogen formulated for immediate release: 40-400 mg of pure or enantiomerically enriched R-BDB, S-BDB, or racemic BDB, and a SSRI formulated for delayed release: 5-40 mg of citalopram;
liv. Entactogen formulated for immediate release: 40-400 mg of pure or enantiomerically enriched R-BDB, S-BDB, or racemic BDB, and a SSRI formulated for delayed release: 5-30 mg of escitalopram;
lv. Entactogen formulated for immediate release: 40-400 mg of pure or enantiomerically enriched R-BDB, S-BDB, or racemic BDB, and a SSRI formulated for delayed release: 10-80 mg of fluoxetine;
lvi. Entactogen formulated for immediate release: 40-400 mg of pure or enantiomerically enriched R-BDB, S-BDB, or racemic BDB, and a SSRI formulated for delayed release: 10-60 mg of paroxetine;
lvii. Entactogen formulated for immediate release: 40-400 mg of pure or enantiomerically enriched R-BDB, S-BDB, or racemic BDB, and a SSRI formulated for delayed release: 20-300 mg of sertraline;
lviii. Entactogen formulated for immediate release: 40-400 mg of pure or enantiomerically enriched R-BDB, S-BDB, or racemic BDB, and a SSRI formulated for delayed release: 20-300 mg of fluvoxamine;
lix. Entactogen formulated for immediate release: 40-400 mg of pure or enantiomerically enriched R-BDB, S-BDB, or racemic BDB, and a SSRI formulated for delayed release: 10-80 mg of vilazodone;
lx. Entactogen formulated for immediate release: 40-400 mg of pure or enantiomerically enriched R-BDB, S-BDB, or racemic BDB, and a SSRI formulated for delayed release: 5-30 mg of vortioxetine;
lxi. Entactogen formulated for immediate release: 40-400 mg of pure or enantiomerically enriched R-BDB, S-BDB, or racemic BDB, and a SNRI formulated for delayed release: 10-120 mg of duloxetine; and
lxii. Entactogen formulated for immediate release: 40-400 mg of pure or enantiomerically enriched R-BDB, S-BDB, or racemic BDB, and a SNRI formulated for delayed release: 5-300 mg of milnacipran.
lxiii. Entactogen formulated for immediate release: 40-400 mg of pure or enantiomerically enriched R-BDB, S-BDB, or racemic BDB, and a SNRI formulated for delayed release: 10-120 mg of levomilnacipran;
lxiv. Entactogen formulated for immediate release: 40-400 mg of pure or enantiomerically enriched R-BDB, S-BDB, or racemic BDB, and a SNRI formulated for delayed release: 15-375 mg of venlafaxine;
lxv. Entactogen formulated for immediate release: 40-400 mg of pure or enantiomerically enriched R-BDB, S-BDB, or racemic BDB, and a SNRI formulated for delayed release: 10-400 mg of desvenlafaxine;
lxvi. Entactogen formulated for immediate release: 20-400 mg of MDAI, and a SSRI formulated for delayed release: 5-40 mg of citalopram;
lxvii. Entactogen formulated for immediate release: 20-400 mg of MDAI, and a SSRI formulated for delayed release: 5-30 mg of escitalopram;
lxviii. Entactogen formulated for immediate release: 20-400 mg of MDAI, and a SSRI formulated for delayed release: 10-80 mg of fluoxetine;
lxix. Entactogen formulated for immediate release: 20-400 mg of MDAI, and a SSRI formulated for delayed release: 10-60 mg of paroxetine;
lxx. Entactogen formulated for immediate release: 20-400 mg of MDAI, and a SSRI formulated for delayed release: 20-300 mg of sertraline;
lxxi. Entactogen formulated for immediate release: 20-400 mg of MDAI, and a SSRI formulated for delayed release: 20-300 mg of fluvoxamine;
lxxii. Entactogen formulated for immediate release: 20-400 mg of MDAI, and a SSRI formulated for delayed release: 10-80 mg of vilazodone;
lxxiii. Entactogen formulated for immediate release: 20-400 mg of MDAI, and a SSRI formulated for delayed release: 5-30 mg of vortioxetine;
lxxiv. Entactogen formulated for immediate release: 20-400 mg of MDAI, and a SNRI formulated for delayed release: 10-120 mg of duloxetine;
lxxv. Entactogen formulated for immediate release: 20-400 mg of MDAI, and a SNRI formulated for delayed release: 5-300 mg of milnacipran;
lxxvi. Entactogen formulated for immediate release: 20-400 mg of MDAI, and a SNRI formulated for delayed release: 10-120 mg of levomilnacipran;
lxxvii. Entactogen formulated for immediate release: 20-400 mg of MDAI, and a SNRI formulated for delayed release: 15-375 mg of venlafaxine;
lxxviii. Entactogen formulated for immediate release: 20-400 mg of MDAI, and a SNRI formulated for delayed release: 10-400 mg of desvenlafaxine;
lxxix. Entactogen formulated for immediate release: 20-400 mg of MDMAI, and a SSRI formulated for delayed release: 5-40 mg of citalopram;
lxxx. Entactogen formulated for immediate release: 20-400 mg of MDMAI, and a SSRI formulated for delayed release: 5-30 mg of escitalopram;
lxxxi. Entactogen formulated for immediate release: 20-400 mg of MDMAI, and a SSRI formulated for delayed release: 10-80 mg of fluoxetine;
lxxxii. Entactogen formulated for immediate release: 20-400 mg of MDMAI, and a SSRI formulated for delayed release: 10-60 mg of paroxetine;
lxxxiii. Entactogen formulated for immediate release: 20-400 mg of MDMAI, and a SSRI formulated for delayed release: 20-300 mg of sertraline;
lxxxiv. Entactogen formulated for immediate release: 20-400 mg of MDMAI, and a SSRI formulated for delayed release: 20-300 mg of fluvoxamine;
lxxxv. Entactogen formulated for immediate release: 20-400 mg of MDMAI, and a SSRI formulated for delayed release: 10-80 mg of vilazodone;
lxxxvi. Entactogen formulated for immediate release: 20-400 mg of MDMAI, and a SSRI formulated for delayed release: 5-30 mg of vortioxetine;
lxxxvii. Entactogen formulated for immediate release: 20-400 mg of MDMAI, and a SNRI formulated for delayed release: 10-120 mg of duloxetine;
lxxxviii. Entactogen formulated for immediate release: 20-400 mg of MDMAI, and a SNRI formulated for delayed release: 5-300 mg of milnacipran;
lxxxix. Entactogen formulated for immediate release: 20-400 mg of MDMAI, and a SNRI formulated for delayed release: 10-120 mg of levomilnacipran;
xc. Entactogen formulated for immediate release: 20-400 mg of MDMAI, and a SNRI formulated for delayed release: 15-375 mg of venlafaxine;
xci. Entactogen formulated for immediate release: 20-400 mg of MDMAI, and a SNRI formulated for delayed release: 10-400 mg of desvenlafaxine;
xcii. Entactogen formulated for immediate release: 20-600 mg of pure or enantiomerically enriched R-MDAT, S-MDAT, or racemic MDAT, and a SSRI formulated for delayed release: 5-40 mg of citalopram;
xciii. Entactogen formulated for immediate release: 20-600 mg of pure or enantiomerically enriched R-MDAT, S-MDAT, or racemic MDAT, and a SSRI formulated for delayed release: 5-30 mg of escitalopram;
xciv. Entactogen formulated for immediate release: 20-600 mg of pure or enantiomerically enriched R-MDAT, S-MDAT, or racemic MDAT, and a SSRI formulated for delayed release: 10-80 mg of fluoxetine;
xcv. Entactogen formulated for immediate release: 20-600 mg of pure or enantiomerically enriched R-MDAT, S-MDAT, or racemic MDAT, and a SSRI formulated for delayed release: 10-60 mg of paroxetine;
xcvi. Entactogen formulated for immediate release: 20-600 mg of pure or enantiomerically enriched R-MDAT, S-MDAT, or racemic MDAT, and a SSRI formulated for delayed release: 20-300 mg of sertraline;
xcvii. Entactogen formulated for immediate release: 20-600 mg of pure or enantiomerically enriched R-MDAT, S-MDAT, or racemic MDAT, and a SSRI formulated for delayed release: 20-300 mg of fluvoxamine;
xcviii. Entactogen formulated for immediate release: 20-600 mg of pure or enantiomerically enriched R-MDAT, S-MDAT, or racemic MDAT, and a SSRI formulated for delayed release: 10-80 mg of vilazodone;
xcix. Entactogen formulated for immediate release: 20-600 mg of pure or enantiomerically enriched R-MDAT, S-MDAT, or racemic MDAT, and a SSRI formulated for delayed release: 5-30 mg of vortioxetine;
c. Entactogen formulated for immediate release: 20-600 mg of pure or enantiomerically enriched R-MDAT, S-MDAT, or racemic MDAT, and a SNRI formulated for delayed release: 10-120 mg of duloxetine; and
ci. Entactogen formulated for immediate release: 20-600 mg of pure or enantiomerically enriched R-MDAT, S-MDAT, or racemic MDAT, and a SNRI formulated for delayed release: 5-300 mg of milnacipran.
cii. Entactogen formulated for immediate release: 20-600 mg of pure or enantiomerically enriched R-MDAT, S-MDAT, or racemic MDAT, and a SNRI formulated for delayed release: 10-120 mg of levomilnacipran;
ciii. Entactogen formulated for immediate release: 20-600 mg of pure or enantiomerically enriched R-MDAT, S-MDAT, or racemic MDAT, and a SNRI formulated for delayed release: 15-375 mg of venlafaxine;
civ. Entactogen formulated for immediate release: 20-600 mg of pure or enantiomerically enriched R-MDAT, S-MDAT, or racemic MDAT, and a SNRI formulated for delayed release: 10-400 mg of desvenlafaxine;
cv. Entactogen formulated for immediate release: 20-600 mg of pure or enantiomerically enriched R-MDMAT, S-MDMAT, or racemic MDMAT, and a SSRI formulated for delayed release: 5-40 mg of citalopram;
cvi. Entactogen formulated for immediate release: 20-600 mg of pure or enantiomerically enriched R-MDMAT, S-MDMAT, or racemic MDMAT, and a SSRI formulated for delayed release: 5-30 mg of escitalopram;
cvii. Entactogen formulated for immediate release: 20-600 mg of pure or enantiomerically enriched R-MDMAT, S-MDMAT, or racemic MDMAT, and a SSRI formulated for delayed release: 10-80 mg of fluoxetine;
cviii. Entactogen formulated for immediate release: 20-600 mg of pure or enantiomerically enriched R-MDMAT, S-MDMAT, or racemic MDMAT, and a SSRI formulated for delayed release: 10-60 mg of paroxetine;
cix. Entactogen formulated for immediate release: 20-600 mg of pure or enantiomerically enriched R-MDMAT, S-MDMAT, or racemic MDMAT, and a SSRI formulated for delayed release: 20-300 mg of sertraline;
cx. Entactogen formulated for immediate release: 20-600 mg of pure or enantiomerically enriched R-MDMAT, S-MDMAT, or racemic MDMAT, and a SSRI formulated for delayed release: 20-300 mg of fluvoxamine;
cxi. Entactogen formulated for immediate release: 20-600 mg of pure or enantiomerically enriched R-MDMAT, S-MDMAT, or racemic MDMAT, and a SSRI formulated for delayed release: 10-80 mg of vilazodone;
cxii. Entactogen formulated for immediate release: 20-600 mg of pure or enantiomerically enriched R-MDMAT, S-MDMAT, or racemic MDMAT, and a SSRI formulated for delayed release: 5-30 mg of vortioxetine;
cxiii. Entactogen formulated for immediate release: 20-600 mg of pure or enantiomerically enriched R-MDMAT, S-MDMAT, or racemic MDMAT, and a SNRI formulated for delayed release: 10-120 mg of duloxetine; and
cxiv. Entactogen formulated for immediate release: 20-600 mg of pure or enantiomerically enriched R-MDMAT, S-MDMAT, or racemic MDMAT, and a SNRI formulated for delayed release: 5-300 mg of milnacipran.
cxv. Entactogen formulated for immediate release: 20-600 mg of pure or enantiomerically enriched R-MDMAT, S-MDMAT, or racemic MDMAT, and a SNRI formulated for delayed release: 10-120 mg of levomilnacipran;
cxvi. Entactogen formulated for immediate release: 20-600 mg of pure or enantiomerically enriched R-MDMAT, S-MDMAT, or racemic MDMAT, and a SNRI formulated for delayed release: 15-375 mg of venlafaxine;
cxvii. Entactogen formulated for immediate release: 20-600 mg of pure or enantiomerically enriched R-MDMAT, S-MDMAT, or racemic MDMAT, and a SNRI formulated for delayed release: 10-400 mg of desvenlafaxine;
cxviii. Entactogen formulated for immediate release: 40-500 mg of pure or enantiomerically enriched R-BK-MDMA, S-BK-MDMA, or racemic BK-MDMA, and a SSRI formulated for delayed release: 5-40 mg of citalopram;
cxix. Entactogen formulated for immediate release: 40-500 mg of pure or enantiomerically enriched R-BK-MDMA, S-BK-MDMA, or racemic BK-MDMA, and a SSRI formulated for delayed release: 5-30 mg of escitalopram;
cxx. Entactogen formulated for immediate release: 40-500 mg of pure or enantiomerically enriched R-BK-MDMA, S-BK-MDMA, or racemic BK-MDMA, and a SSRI formulated for delayed release: 10-80 mg of fluoxetine;
cxxi. Entactogen formulated for immediate release: 40-500 mg of pure or enantiomerically enriched R-BK-MDMA, S-BK-MDMA, or racemic BK-MDMA, and a SSRI formulated for delayed release: 10-60 mg of paroxetine;
cxxii. Entactogen formulated for immediate release: 40-500 mg of pure or enantiomerically enriched R-BK-MDMA, S-BK-MDMA, or racemic BK-MDMA, and a SSRI formulated for delayed release: 20-300 mg of sertraline;
cxxiii. Entactogen formulated for immediate release: 40-500 mg of pure or enantiomerically enriched R-BK-MDMA, S-BK-MDMA, or racemic BK-MDMA, and a SSRI formulated for delayed release: 20-300 mg of fluvoxamine;
cxxiv. Entactogen formulated for immediate release: 40-500 mg of pure or enantiomerically enriched R-BK-MDMA, S-BK-MDMA, or racemic BK-MDMA, and a SSRI formulated for delayed release: 10-80 mg of vilazodone;
cxxv. Entactogen formulated for immediate release: 40-500 mg of pure or enantiomerically enriched R-BK-MDMA, S-BK-MDMA, or racemic BK-MDMA, and a SSRI formulated for delayed release: 5-30 mg of vortioxetine;
cxxvi. Entactogen formulated for immediate release: 40-500 mg of pure or enantiomerically enriched R-BK-MDMA, S-BK-MDMA, or racemic BK-MDMA, and a SNRI formulated for delayed release: 10-120 mg of duloxetine; and
cxxvii. Entactogen formulated for immediate release: 40-500 mg of pure or enantiomerically enriched R-BK-MDMA, S-BK-MDMA, or racemic BK-MDMA, and a SNRI formulated for delayed release: 5-300 mg of milnacipran.
cxxviii. Entactogen formulated for immediate release: 40-500 mg of pure or enantiomerically enriched R-BK-MDMA, S-BK-MDMA, or racemic BK-MDMA, and a SNRI formulated for delayed release: 10-120 mg of levomilnacipran;
cxxix. Entactogen formulated for immediate release: 40-500 mg of pure or enantiomerically enriched R-BK-MDMA, S-BK-MDMA, or racemic BK-MDMA, and a SNRI formulated for delayed release: 15-375 mg of venlafaxine;
cxxx. Entactogen formulated for immediate release: 40-500 mg of pure or enantiomerically enriched R-BK-MDMA, S-BK-MDMA, or racemic BK-MDMA, and a SNRI formulated for delayed release: 10-400 mg of desvenlafaxine;
cxxxi. Entactogen formulated for immediate release: 40-500 mg of pure or enantiomerically enriched R-BK-MDEA, S-BK-MDEA, or racemic BK-MDEA, and a SSRI formulated for delayed release: 5-40 mg of citalopram;
cxxxii. Entactogen formulated for immediate release: 40-500 mg of pure or enantiomerically enriched R-BK-MDEA, S-BK-MDEA, or racemic BK-MDEA, and a SSRI formulated for delayed release: 5-30 mg of escitalopram;
cxxxiii. Entactogen formulated for immediate release: 40-500 mg of pure or enantiomerically enriched R-BK-MDEA, S-BK-MDEA, or racemic BK-MDEA, and a SSRI formulated for delayed release: 10-80 mg of fluoxetine;
cxxxiv. Entactogen formulated for immediate release: 40-500 mg of pure or enantiomerically enriched R-BK-MDEA, S-BK-MDEA, or racemic BK-MDEA, and a SSRI formulated for delayed release: 10-60 mg of paroxetine;
cxxxv. Entactogen formulated for immediate release: 40-500 mg of pure or enantiomerically enriched R-BK-MDEA, S-BK-MDEA, or racemic BK-MDEA, and a SSRI formulated for delayed release: 20-300 mg of sertraline;
cxxxvi. Entactogen formulated for immediate release: 40-500 mg of pure or enantiomerically enriched R-BK-MDEA, S-BK-MDEA, or racemic BK-MDEA, and a SSRI formulated for delayed release: 20-300 mg of fluvoxamine;
cxxxvii. Entactogen formulated for immediate release: 40-500 mg of pure or enantiomerically enriched R-BK-MDEA, S-BK-MDEA, or racemic BK-MDEA, and a SSRI formulated for delayed release: 10-80 mg of vilazodone;
cxxxviii. Entactogen formulated for immediate release: 40-500 mg of pure or enantiomerically enriched R-BK-MDEA, S-BK-MDEA, or racemic BK-MDEA, and a SSRI formulated for delayed release: 5-30 mg of vortioxetine;
cxxxix. Entactogen formulated for immediate release: 40-500 mg of pure or enantiomerically enriched R-BK-MDEA, S-BK-MDEA, or racemic BK-MDEA, and a SNRI formulated for delayed release: 10-120 mg of duloxetine; and
cxl. Entactogen formulated for immediate release: 40-500 mg of pure or enantiomerically enriched R-BK-MDEA, S-BK-MDEA, or racemic BK-MDEA, and a SNRI formulated for delayed release: 5-300 mg of milnacipran.
cxli. Entactogen formulated for immediate release: 40-500 mg of pure or enantiomerically enriched R-BK-MDEA, S-BK-MDEA, or racemic BK-MDEA, and a SNRI formulated for delayed release: 10-120 mg of levomilnacipran;
cxlii. Entactogen formulated for immediate release: 40-500 mg of pure or enantiomerically enriched R-BK-MDEA, S-BK-MDEA, or racemic BK-MDEA, and a SNRI formulated for delayed release: 15-375 mg of venlafaxine;
cxliii. Entactogen formulated for immediate release: 40-500 mg of pure or enantiomerically enriched R-BK-MDEA, S-BK-MDEA, or racemic BK-MDEA, and a SNRI formulated for delayed release: 10-400 mg of desvenlafaxine;
cxliv. Entactogen formulated for immediate release: 10-200 mg of pure or enantiomerically enriched R-5-APB, S-5-APB, or racemic 5-APB, and a SSRI formulated for delayed release: 5-40 mg of citalopram;
cxlv. Entactogen formulated for immediate release: 10-200 mg of pure or enantiomerically enriched R-5-APB, S-5-APB, or racemic 5-APB, and a SSRI formulated for delayed release: 5-30 mg of escitalopram;
cxlvi. Entactogen formulated for immediate release: 10-200 mg of pure or enantiomerically enriched R-5-APB, S-5-APB, or racemic 5-APB, and a SSRI formulated for delayed release: 10-80 mg of fluoxetine;
cxlvii. Entactogen formulated for immediate release: 10-200 mg of pure or enantiomerically enriched R-5-APB, S-5-APB, or racemic 5-APB, and a SSRI formulated for delayed release: 10-60 mg of paroxetine;
cxlviii. Entactogen formulated for immediate release: 10-200 mg of pure or enantiomerically enriched R-5-APB, S-5-APB, or racemic 5-APB, and a SSRI formulated for delayed release: 20-300 mg of sertraline;
cxlix. Entactogen formulated for immediate release: 10-200 mg of pure or enantiomerically enriched R-5-APB, S-5-APB, or racemic 5-APB, and a SSRI formulated for delayed release: 20-300 mg of fluvoxamine;
cl. Entactogen formulated for immediate release: 10-200 mg of pure or enantiomerically enriched R-5-APB, S-5-APB, or racemic 5-APB, and a SSRI formulated for delayed release: 10-80 mg of vilazodone;
cli. Entactogen formulated for immediate release: 10-200 mg of pure or enantiomerically enriched R-5-APB, S-5-APB, or racemic 5-APB, and a SSRI formulated for delayed release: 5-30 mg of vortioxetine;
clii. Entactogen formulated for immediate release: 10-200 mg of pure or enantiomerically enriched R-5-APB, S-5-APB, or racemic 5-APB, and a SNRI formulated for delayed release: 10-120 mg of duloxetine; and
cliii. Entactogen formulated for immediate release: 10-200 mg of pure or enantiomerically enriched R-5-APB, S-5-APB, or racemic 5-APB, and a SNRI formulated for delayed release: 5-300 mg of milnacipran.
cliv. Entactogen formulated for immediate release: 10-200 mg of pure or enantiomerically enriched R-5-APB, S-5-APB, or racemic 5-APB, and a SNRI formulated for delayed release: 10-120 mg of levomilnacipran;
clv. Entactogen formulated for immediate release: 10-200 mg of pure or enantiomerically enriched R-5-APB, S-5-APB, or racemic 5-APB, and a SNRI formulated for delayed release: 15-375 mg of venlafaxine;
clvi. Entactogen formulated for immediate release: 10-200 mg of pure or enantiomerically enriched R-5-APB, S-5-APB, or racemic 5-APB, and a SNRI formulated for delayed release: 10-400 mg of desvenlafaxine;
clvii. Entactogen formulated for immediate release: 10-200 mg of pure or enantiomerically enriched R-6-APB, S-6-APB, or racemic 6-APB, and a SSRI formulated for delayed release: 5-40 mg of citalopram;
clviii. Entactogen formulated for immediate release: 10-200 mg of pure or enantiomerically enriched R-6-APB, S-6-APB, or racemic 6-APB, and a SSRI formulated for delayed release: 5-30 mg of escitalopram;
clix. Entactogen formulated for immediate release: 10-200 mg of pure or enantiomerically enriched R-6-APB, S-6-APB, or racemic 6-APB, and a SSRI formulated for delayed release: 10-80 mg of fluoxetine;
clx. Entactogen formulated for immediate release: 10-200 mg of pure or enantiomerically enriched R-6-APB, S-6-APB, or racemic 6-APB, and a SSRI formulated for delayed release: 10-60 mg of paroxetine;
clxi. Entactogen formulated for immediate release: 10-200 mg of pure or enantiomerically enriched R-6-APB, S-6-APB, or racemic 6-APB, and a SSRI formulated for delayed release: 20-300 mg of sertraline;
clxii. Entactogen formulated for immediate release: 10-200 mg of pure or enantiomerically enriched R-6-APB, S-6-APB, or racemic 6-APB, and a SSRI formulated for delayed release: 20-300 mg of fluvoxamine;
clxiii. Entactogen formulated for immediate release: 10-200 mg of pure or enantiomerically enriched R-6-APB, S-6-APB, or racemic 6-APB, and a SSRI formulated for delayed release: 10-80 mg of vilazodone;
clxiv. Entactogen formulated for immediate release: 10-200 mg of pure or enantiomerically enriched R-6-APB, S-6-APB, or racemic 6-APB, and a SSRI formulated for delayed release: 5-30 mg of vortioxetine;
clxv. Entactogen formulated for immediate release: 10-200 mg of pure or enantiomerically enriched R-6-APB, S-6-APB, or racemic 6-APB, and a SNRI formulated for delayed release: 10-120 mg of duloxetine; and
clxvi. Entactogen formulated for immediate release: 10-200 mg of pure or enantiomerically enriched R-6-APB, S-6-APB, or racemic 6-APB, and a SNRI formulated for delayed release: 5-300 mg of milnacipran.
clxvii. Entactogen formulated for immediate release: 10-200 mg of pure or enantiomerically enriched R-6-APB, S-6-APB, or racemic 6-APB, and a SNRI formulated for delayed release: 10-120 mg of levomilnacipran;
clxviii. Entactogen formulated for immediate release: 10-200 mg of pure or enantiomerically enriched R-6-APB, S-6-APB, or racemic 6-APB, and a SNRI formulated for delayed release: 15-375 mg of venlafaxine;
clxix. Entactogen formulated for immediate release: 10-200 mg of pure or enantiomerically enriched R-6-APB, S-6-APB, or racemic 6-APB, and a SNRI formulated for delayed release: 10-400 mg of desvenlafaxine;
clxx. Entactogen formulated for immediate release: 10-200 mg of pure or enantiomerically enriched R-5-MAPB, S-5-MAPB, or racemic 5-MAPB, and a SSRI formulated for delayed release: 5-40 mg of citalopram;
clxxi. Entactogen formulated for immediate release: 10-200 mg of pure or enantiomerically enriched R-5-MAPB, S-5-MAPB, or racemic 5-MAPB, and a SSRI formulated for delayed release: 5-30 mg of escitalopram;
clxxii. Entactogen formulated for immediate release: 10-200 mg of pure or enantiomerically enriched R-5-MAPB, S-5-MAPB, or racemic 5-MAPB, and a SSRI formulated for delayed release: 10-80 mg of fluoxetine;
clxxiii. Entactogen formulated for immediate release: 10-200 mg of pure or enantiomerically enriched R-5-MAPB, S-5-MAPB, or racemic 5-MAPB, and a SSRI formulated for delayed release: 10-60 mg of paroxetine;
clxxiv. Entactogen formulated for immediate release: 10-200 mg of pure or enantiomerically enriched R-5-MAPB, S-5-MAPB, or racemic 5-MAPB, and a SSRI formulated for delayed release: 20-300 mg of sertraline;
clxxv. Entactogen formulated for immediate release: 10-200 mg of pure or enantiomerically enriched R-5-MAPB, S-5-MAPB, or racemic 5-MAPB, and a SSRI formulated for delayed release: 20-300 mg of fluvoxamine;
clxxvi. Entactogen formulated for immediate release: 10-200 mg of pure or enantiomerically enriched R-5-MAPB, S-5-MAPB, or racemic 5-MAPB, and a SSRI formulated for delayed release: 10-80 mg of vilazodone;
clxxvii. Entactogen formulated for immediate release: 10-200 mg of pure or enantiomerically enriched R-5-MAPB, S-5-MAPB, or racemic 5-MAPB, and a SSRI formulated for delayed release: 5-30 mg of vortioxetine;
clxxviii. Entactogen formulated for immediate release: 10-200 mg of pure or enantiomerically enriched R-5-MAPB, S-5-MAPB, or racemic 5-MAPB, and a SNRI formulated for delayed release: 10-120 mg of duloxetine; and
clxxix. Entactogen formulated for immediate release: 10-200 mg of pure or enantiomerically enriched R-5-MAPB, S-5-MAPB, or racemic 5-MAPB, and a SNRI formulated for delayed release: 5-300 mg of milnacipran.
clxxx. Entactogen formulated for immediate release: 10-200 mg of pure or enantiomerically enriched R-5-MAPB, S-5-MAPB, or racemic 5-MAPB, and a SNRI formulated for delayed release: 10-120 mg of levomilnacipran;
clxxxi. Entactogen formulated for immediate release: 10-200 mg of pure or enantiomerically enriched R-5-MAPB, S-5-MAPB, or racemic 5-MAPB, and a SNRI formulated for delayed release: 15-375 mg of venlafaxine;
clxxxii. Entactogen formulated for immediate release: 10-200 mg of pure or enantiomerically enriched R-5-MAPB, S-5-MAPB, or racemic 5-MAPB, and a SNRI formulated for delayed release: 10-400 mg of desvenlafaxine;
clxxxiii. Entactogen formulated for immediate release: 10-200 mg of pure or enantiomerically enriched R-6-MAPB, S-6-MAPB, or racemic 6-MAPB, and a SSRI formulated for delayed release: 5-40 mg of citalopram;
clxxxiv. Entactogen formulated for immediate release: 10-200 mg of pure or enantiomerically enriched R-6-MAPB, S-6-MAPB, or racemic 6-MAPB, and a SSRI formulated for delayed release: 5-30 mg of escitalopram;
clxxxv. Entactogen formulated for immediate release: 10-200 mg of pure or enantiomerically enriched R-6-MAPB, S-6-MAPB, or racemic 6-MAPB, and a SSRI formulated for delayed release: 10-80 mg of fluoxetine;
clxxxvi. Entactogen formulated for immediate release: 10-200 mg of pure or enantiomerically enriched R-6-MAPB, S-6-MAPB, or racemic 6-MAPB, and a SSRI formulated for delayed release: 10-60 mg of paroxetine;
clxxxvii. Entactogen formulated for immediate release: 10-200 mg of pure or enantiomerically enriched R-6-MAPB, S-6-MAPB, or racemic 6-MAPB, and a SSRI formulated for delayed release: 20-300 mg of sertraline;
clxxxviii. Entactogen formulated for immediate release: 10-200 mg of pure or enantiomerically enriched R-6-MAPB, S-6-MAPB, or racemic 6-MAPB, and a SSRI formulated for delayed release: 20-300 mg of fluvoxamine;
clxxxix. Entactogen formulated for immediate release: 10-200 mg of pure or enantiomerically enriched R-6-MAPB, S-6-MAPB, or racemic 6-MAPB, and a SSRI formulated for delayed release: 10-80 mg of vilazodone;
cxc. Entactogen formulated for immediate release: 10-200 mg of pure or enantiomerically enriched R-6-MAPB, S-6-MAPB, or racemic 6-MAPB, and a SSRI formulated for delayed release: 5-30 mg of vortioxetine;
cxci. Entactogen formulated for immediate release: 10-200 mg of pure or enantiomerically enriched R-6-MAPB, S-6-MAPB, or racemic 6-MAPB, and a SNRI formulated for delayed release: 10-120 mg of duloxetine; and
cxcii. Entactogen formulated for immediate release: 10-200 mg of pure or enantiomerically enriched R-6-MAPB, S-6-MAPB, or racemic 6-MAPB, and a SNRI formulated for delayed release: 5-300 mg of milnacipran.
cxciii. Entactogen formulated for immediate release: 10-200 mg of pure or enantiomerically enriched R-6-MAPB, S-6-MAPB, or racemic 6-MAPB, and a SNRI formulated for delayed release: 10-120 mg of levomilnacipran;
cxciv. Entactogen formulated for immediate release: 10-200 mg of pure or enantiomerically enriched R-6-MAPB, S-6-MAPB, or racemic 6-MAPB, and a SNRI formulated for delayed release: 15-375 mg of venlafaxine; and
cxcv. Entactogen formulated for immediate release: 10-200 mg of pure or enantiomerically enriched R-6-MAPB, S-6-MAPB, or racemic 6-MAPB, and a SNRI formulated for delayed release: 10-400 mg of desvenlafaxine.
cxcvi. Entactogen formulated for immediate release: 5-200 mg of pure or enantiomerically enriched R-BK-5-MAPB, S-BK-5-MAPB, or racemic BK-5-MAPB, and a SSRI formulated for delayed release: 5-40 mg of citalopram;
cxcvii. Entactogen formulated for immediate release: 5-200 mg of pure or enantiomerically enriched R-BK-5-MAPB, S-BK-5-MAPB, or racemic BK-5-MAPB, and a SSRI formulated for delayed release: 5-30 mg of escitalopram;
cxcviii. Entactogen formulated for immediate release: 5-200 mg of pure or enantiomerically enriched R-BK-5-MAPB, S-BK-5-MAPB, or racemic BK-5-MAPB, and a SSRI formulated for delayed release: 10-80 mg of fluoxetine;
cxcix. Entactogen formulated for immediate release: 5-200 mg of pure or enantiomerically enriched R-BK-5-MAPB, S-BK-5-MAPB, or racemic BK-5-MAPB, and a SSRI formulated for delayed release: 10-60 mg of paroxetine;
cc. Entactogen formulated for immediate release: 5-200 mg of pure or enantiomerically enriched R-BK-5-MAPB, S-BK-5-MAPB, or racemic BK-5-MAPB, and a SSRI formulated for delayed release: 20-300 mg of sertraline;
cci. Entactogen formulated for immediate release: 5-200 mg of pure or enantiomerically enriched R-BK-5-MAPB, S-BK-5-MAPB, or racemic BK-5-MAPB, and a SSRI formulated for delayed release: 20-300 mg of fluvoxamine;
ccii. Entactogen formulated for immediate release: 5-200 mg of pure or enantiomerically enriched R-BK-5-MAPB, S-BK-5-MAPB, or racemic BK-5-MAPB, and a SSRI formulated for delayed release: 10-80 mg of vilazodone;
cciii. Entactogen formulated for immediate release: 5-200 mg of pure or enantiomerically enriched R-BK-5-MAPB, S-BK-5-MAPB, or racemic BK-5-MAPB, and a SSRI formulated for delayed release: 5-30 mg of vortioxetine;
cciv. Entactogen formulated for immediate release: 5-200 mg of pure or enantiomerically enriched R-BK-5-MAPB, S-BK-5-MAPB, or racemic BK-5-MAPB, and a SNRI formulated for delayed release: 10-120 mg of duloxetine; and
ccv. Entactogen formulated for immediate release: 5-200 mg of pure or enantiomerically enriched R-BK-5-MAPB, S-BK-5-MAPB, or racemic BK-5-MAPB, and a SNRI formulated for delayed release: 5-300 mg of milnacipran.
ccvi. Entactogen formulated for immediate release: 5-200 mg of pure or enantiomerically enriched R-BK-5-MAPB, S-BK-5-MAPB, or racemic BK-5-MAPB, and a SNRI formulated for delayed release: 10-120 mg of levomilnacipran;
ccvii. Entactogen formulated for immediate release: 5-200 mg of pure or enantiomerically enriched R-BK-5-MAPB, S-BK-5-MAPB, or racemic BK-5-MAPB, and a SNRI formulated for delayed release: 15-375 mg of venlafaxine; and
ccviii. Entactogen formulated for immediate release: 5-200 mg of pure or enantiomerically enriched R-BK-5-MAPB, S-BK-5-MAPB, or racemic BK-5-MAPB, and a SNRI formulated for delayed release: 10-400 mg of desvenlafaxine.
ccix. Entactogen formulated for immediate release: 5-200 mg of pure or enantiomerically enriched R-BK-5-EAPB, S-BK-5-EAPB, or racemic BK-5-EAPB, and a SSRI formulated for delayed release: 5-40 mg of citalopram;
ccx. Entactogen formulated for immediate release: 5-200 mg of pure or enantiomerically enriched R-BK-5-EAPB, S-BK-5-EAPB, or racemic BK-5-EAPB, and a SSRI formulated for delayed release: 5-30 mg of escitalopram;
ccxi. Entactogen formulated for immediate release: 5-200 mg of pure or enantiomerically enriched R-BK-5-EAPB, S-BK-5-EAPB, or racemic BK-5-EAPB, and a SSRI formulated for delayed release: 10-80 mg of fluoxetine;
ccxii. Entactogen formulated for immediate release: 5-200 mg of pure or enantiomerically enriched R-BK-5-EAPB, S-BK-5-EAPB, or racemic BK-5-EAPB, and a SSRI formulated for delayed release: 10-60 mg of paroxetine;
ccxiii. Entactogen formulated for immediate release: 5-200 mg of pure or enantiomerically enriched R-BK-5-EAPB, S-BK-5-EAPB, or racemic BK-5-EAPB, and a SSRI formulated for delayed release: 20-300 mg of sertraline;
ccxiv. Entactogen formulated for immediate release: 5-200 mg of pure or enantiomerically enriched R-BK-5-EAPB, S-BK-5-EAPB, or racemic BK-5-EAPB, and a SSRI formulated for delayed release: 20-300 mg of fluvoxamine;
ccxv. Entactogen formulated for immediate release: 5-200 mg of pure or enantiomerically enriched R-BK-5-EAPB, S-BK-5-EAPB, or racemic BK-5-EAPB, and a SSRI formulated for delayed release: 10-80 mg of vilazodone;
ccxvi. Entactogen formulated for immediate release: 5-200 mg of pure or enantiomerically enriched R-BK-5-EAPB, S-BK-5-EAPB, or racemic BK-5-EAPB, and a SSRI formulated for delayed release: 5-30 mg of vortioxetine;
ccxvii. Entactogen formulated for immediate release: 5-200 mg of pure or enantiomerically enriched R-BK-5-EAPB, S-BK-5-EAPB, or racemic BK-5-EAPB, and a SNRI formulated for delayed release: 10-120 mg of duloxetine; and
ccxviii. Entactogen formulated for immediate release: 5-200 mg of pure or enantiomerically enriched R-BK-5-EAPB, S-BK-5-EAPB, or racemic BK-5-EAPB, and a SNRI formulated for delayed release: 5-300 mg of milnacipran.
ccxix. Entactogen formulated for immediate release: 5-200 mg of pure or enantiomerically enriched R-BK-5-EAPB, S-BK-5-EAPB, or racemic BK-5-EAPB, and a SNRI formulated for delayed release: 10-120 mg of levomilnacipran;
ccxx. Entactogen formulated for immediate release: 5-200 mg of pure or enantiomerically enriched R-BK-5-EAPB, S-BK-5-EAPB, or racemic BK-5-EAPB, and a SNRI formulated for delayed release: 15-375 mg of venlafaxine; and
ccxxi. Entactogen formulated for immediate release: 5-200 mg of pure or enantiomerically enriched R-BK-5-EAPB, S-BK-5-EAPB, or racemic BK-5-EAPB, and a SNRI formulated for delayed release: 10-400 mg of desvenlafaxine.

Additional non-limiting exemplary formulations of the present invention include
i. Entactogen formulated for immediate release: 10-200 mg of pure or enantiomerically enriched R-MDMA, S-MDMA, or racemic MDMA, and a SSRI formulated for delayed release: 2-20 mg of citalopram;
ii. Entactogen formulated for immediate release: 10-200 mg of pure or enantiomerically enriched R-MDMA, S-MDMA, or racemic MDMA, and a SSRI formulated for delayed release: 2-15 mg of escitalopram;
iii. Entactogen formulated for immediate release: 10-200 mg of pure or enantiomerically enriched R-MDMA, S-MDMA, or racemic MDMA, and a SSRI formulated for delayed release: 5-40 mg of fluoxetine;
iv. Entactogen formulated for immediate release: 10-200 mg of pure or enantiomerically enriched R-MDMA, S-MDMA, or racemic MDMA, and a SSRI formulated for delayed release: 5-30 mg of paroxetine;
v. Entactogen formulated for immediate release: 10-200 mg of pure or enantiomerically enriched R-MDMA, S-MDMA, or racemic MDMA, and a SSRI formulated for delayed release: 10-150 mg of sertraline;
vi. Entactogen formulated for immediate release: 10-200 mg of pure or enantiomerically enriched R-MDMA, S-MDMA, or racemic MDMA, and a SSRI formulated for delayed release: 10-150 mg of fluvoxamine;
vii. Entactogen formulated for immediate release: 10-200 mg of pure or enantiomerically enriched R-MDMA, S-MDMA, or racemic MDMA, and a SSRI formulated for delayed release: 10-80 mg of vilazodone;
viii. Entactogen formulated for immediate release: 10-200 mg of pure or enantiomerically enriched R-MDMA, S-MDMA, or racemic MDMA, and a SSRI formulated for delayed release: 2-15 mg of vortioxetine;
ix. Entactogen formulated for immediate release: 10-200 mg of pure or enantiomerically enriched R-MDMA, S-MDMA, or racemic MDMA, and a SNRI formulated for delayed release: 5-60 mg of duloxetine;
x. Entactogen formulated for immediate release: 10-200 mg of pure or enantiomerically enriched R-MDMA, S-MDMA, or racemic MDMA, and a SNRI formulated for delayed release: 2-150 mg of milnacipran;
xi. Entactogen formulated for immediate release: 10-200 mg of pure or enantiomerically enriched R-MDMA, S-MDMA, or racemic MDMA, and a SNRI formulated for delayed release: 5-60 mg of levomilnacipran;
xii. Entactogen formulated for immediate release: 10-200 mg of pure or enantiomerically enriched R-MDMA, S-MDMA, or racemic MDMA, and a SNRI formulated for delayed release: 5-200 mg of venlafaxine;
xiii. Entactogen formulated for immediate release: 10-200 mg of pure or enantiomerically enriched R-MDMA, S-MDMA, or racemic MDMA, and a SNRI formulated for delayed release: 5-200 mg of desvenlafaxine;
xiv. Entactogen formulated for immediate release: 10-150 mg of pure or enantiomerically enriched R-MDA, S-MDA, or racemic MDA, and a SSRI formulated for delayed release: 5-40 mg of citalopram;
xv. Entactogen formulated for immediate release: 10-150 mg of pure or enantiomerically enriched R-MDA, S-MDA, or racemic MDA, and a SSRI formulated for delayed release: 2-15 mg of escitalopram;
xvi. Entactogen formulated for immediate release: 10-150 mg of pure or enantiomerically enriched R-MDA, S-MDA, or racemic MDA, and a SSRI formulated for delayed release: 10-80 mg of fluoxetine;
xvii. Entactogen formulated for immediate release: 10-150 mg of pure or enantiomerically enriched R-MDA, S-MDA, or racemic MDA, and a SSRI formulated for delayed release: 5-30 mg of paroxetine;
xviii. Entactogen formulated for immediate release: 10-150 mg of pure or enantiomerically enriched R-MDA, S-MDA, or racemic MDA, and a SSRI formulated for delayed release: 10-150 mg of sertraline;
xix. Entactogen formulated for immediate release: 10-150 mg of pure or enantiomerically enriched R-MDA, S-MDA, or racemic MDA, and a SSRI formulated for delayed release: 10-150 mg of fluvoxamine;
xx. Entactogen formulated for immediate release: 10-150 mg of pure or enantiomerically enriched R-MDA, S-MDA, or racemic MDA, and a SSRI formulated for delayed release: 5-40 mg of vilazodone;
xxi. Entactogen formulated for immediate release: 10-150 mg of pure or enantiomerically enriched R-MDA, S-MDA, or racemic MDA, and a SSRI formulated for delayed release: 2-15 mg of vortioxetine;
xxii. Entactogen formulated for immediate release: 10-150 mg of pure or enantiomerically enriched R-MDA, S-MDA, or racemic MDA, and a SNRI formulated for delayed release: 5-60 mg of duloxetine; and
xxiii. Entactogen formulated for immediate release: 10-150 mg of pure or enantiomerically enriched R-MDA, S-MDA, or racemic MDA, and a SNRI formulated for delayed release: 2-150 mg of milnacipran.
xxiv. Entactogen formulated for immediate release: 10-150 mg of pure or enantiomerically enriched R-MDA, S-MDA, or racemic MDA, and a SNRI formulated for delayed release: 5-60 mg of levomilnacipran;
xxv. Entactogen formulated for immediate release: 10-150 mg of pure or enantiomerically enriched R-MDA, S-MDA, or racemic MDA, and a SNRI formulated for delayed release: 5-200 mg of venlafaxine;
xxvi. Entactogen formulated for immediate release: 10-150 mg of pure or enantiomerically enriched R-MDA, S-MDA, or racemic MDA, and a SNRI formulated for delayed release: 5-200 mg of desvenlafaxine;
xxvii. Entactogen formulated for immediate release: 10-250 mg of pure or enantiomerically enriched R-MDE, S-MDE, or racemic MDE, and a SSRI formulated for delayed release: 2-20 mg of citalopram;
xxviii. Entactogen formulated for immediate release: 10-250 mg of pure or enantiomerically enriched R-MDE, S-MDE, or racemic MDE, and a SSRI formulated for delayed release: 2-15 mg of escitalopram;
xxix. Entactogen formulated for immediate release: 10-250 mg of pure or enantiomerically enriched R-MDE, S-MDE, or racemic MDE, and a SSRI formulated for delayed release: 5-40 mg of fluoxetine;
xxx. Entactogen formulated for immediate release: 10-250 mg of pure or enantiomerically enriched R-MDE, S-MDE, or racemic MDE, and a SSRI formulated for delayed release: 5-30 mg of paroxetine;
xxxi. Entactogen formulated for immediate release: 10-250 mg of pure or enantiomerically enriched R-MDE, S-MDE, or racemic MDE, and a SSRI formulated for delayed release: 10-150 mg of sertraline;
xxxii. Entactogen formulated for immediate release: 10-250 mg of pure or enantiomerically enriched R-MDE, S-MDE, or racemic MDE, and a SSRI formulated for delayed release: 10-150 mg of fluvoxamine;
xxxiii. Entactogen formulated for immediate release: 10-250 mg of pure or enantiomerically enriched R-MDE, S-MDE, or racemic MDE, and a SSRI formulated for delayed release: 5-40 mg of vilazodone;
xxxiv. Entactogen formulated for immediate release: 10-250 mg of pure or enantiomerically enriched R-MDE, S-MDE, or racemic MDE, and a SSRI formulated for delayed release: 2-15 mg of vortioxetine;
xxxv. Entactogen formulated for immediate release: 10-250 mg of pure or enantiomerically enriched R-MDE, S-MDE, or racemic MDE, and a SNRI formulated for delayed release: 2-60 mg of duloxetine; and
xxxvi. Entactogen formulated for immediate release: 10-250 mg of pure or enantiomerically enriched R-MDE, S-MDE, or racemic MDE, and a SNRI formulated for delayed release: 2-150 mg of milnacipran.
xxxvii. Entactogen formulated for immediate release: 10-250 mg of pure or enantiomerically enriched R-MDE, S-MDE, or racemic MDE, and a SNRI formulated for delayed release: 5-60 mg of levomilnacipran;
xxxviii. Entactogen formulated for immediate release: 10-250 mg of pure or enantiomerically enriched R-MDE, S-MDE, or racemic MDE, and a SNRI formulated for delayed release: 5-200 mg of venlafaxine;
xxxix. Entactogen formulated for immediate release: 10-250 mg of pure or enantiomerically enriched R-MDE, S-MDE, or racemic MDE, and a SNRI formulated for delayed release: 5-200 mg of desvenlafaxine;
xl. Entactogen formulated for immediate release: 20-200 mg of pure or enantiomerically enriched R-MBDB, S-MBDB, or racemic MBDB, and a SSRI formulated for delayed release: 2-20 mg of citalopram;
xli. Entactogen formulated for immediate release: 20-200 mg of pure or enantiomerically enriched R-MBDB, S-MBDB, or racemic MBDB, and a SSRI formulated for delayed release: 2-15 mg of escitalopram;
xlii. Entactogen formulated for immediate release: 20-200 mg of pure or enantiomerically enriched R-MBDB, S-MBDB, or racemic MBDB, and a SSRI formulated for delayed release: 5-40 mg of fluoxetine;
xliii. Entactogen formulated for immediate release: 20-200 mg of pure or enantiomerically enriched R-MBDB, S-MBDB, or racemic MBDB, and a SSRI formulated for delayed release: 5-30 mg of paroxetine;
xliv. Entactogen formulated for immediate release: 20-200 mg of pure or enantiomerically enriched R-MBDB, S-MBDB, or racemic MBDB, and a SSRI formulated for delayed release: 10-150 mg of sertraline;
xlv. Entactogen formulated for immediate release: 20-200 mg of pure or enantiomerically enriched R-MBDB, S-MBDB, or racemic MBDB, and a SSRI formulated for delayed release: 10-150 mg of fluvoxamine;
xlvi. Entactogen formulated for immediate release: 20-200 mg of pure or enantiomerically enriched R-MBDB, S-MBDB, or racemic MBDB, and a SSRI formulated for delayed release: 5-40 mg of vilazodone;
xlvii. Entactogen formulated for immediate release: 20-200 mg of pure or enantiomerically enriched R-MBDB, S-MBDB, or racemic MBDB, and a SSRI formulated for delayed release: 2-15 mg of vortioxetine;
xlviii. Entactogen formulated for immediate release: 20-200 mg of pure or enantiomerically enriched R-MBDB, S-MBDB, or racemic MBDB, and a SNRI formulated for delayed release: 5-60 mg of duloxetine; and
xlix. Entactogen formulated for immediate release: 20-200 mg of pure or enantiomerically enriched R-MBDB, S-MBDB, or racemic MBDB, and a SNRI formulated for delayed release: 2-150 mg of milnacipran.
l. Entactogen formulated for immediate release: 20-200 mg of pure or enantiomerically enriched R-MBDB, S-MBDB, or racemic MBDB, and a SNRI formulated for delayed release: 5-60 mg of levomilnacipran;
li. Entactogen formulated for immediate release: 20-200 mg of pure or enantiomerically enriched R-MBDB, S-MBDB, or racemic MBDB, and a SNRI formulated for delayed release: 5-200 mg of venlafaxine;
lii. Entactogen formulated for immediate release: 20-200 mg of pure or enantiomerically enriched R-MBDB, S-MBDB, or racemic MBDB, and a SNRI formulated for delayed release: 5-200 mg of desvenlafaxine;
liii. Entactogen formulated for immediate release: 20-200 mg of pure or enantiomerically enriched R-BDB, S-BDB, or racemic BDB, and a SSRI formulated for delayed release: 2-20 mg of citalopram;
liv. Entactogen formulated for immediate release: 20-200 mg of pure or enantiomerically enriched R-BDB, S-BDB, or racemic BDB, and a SSRI formulated for delayed release: 2-15 mg of escitalopram;
lv. Entactogen formulated for immediate release: 20-200 mg of pure or enantiomerically enriched R-BDB, S-BDB, or racemic BDB, and a SSRI formulated for delayed release: 5-40 mg of fluoxetine;
lvi. Entactogen formulated for immediate release: 20-200 mg of pure or enantiomerically enriched R-BDB, S-BDB, or racemic BDB, and a SSRI formulated for delayed release: 5-30 mg of paroxetine;
lvii. Entactogen formulated for immediate release: 20-200 mg of pure or enantiomerically enriched R-BDB, S-BDB, or racemic BDB, and a SSRI formulated for delayed release: 10-150 mg of sertraline;
lviii. Entactogen formulated for immediate release: 20-200 mg of pure or enantiomerically enriched R-BDB, S-BDB, or racemic BDB, and a SSRI formulated for delayed release: 10-150 mg of fluvoxamine;
lix. Entactogen formulated for immediate release: 20-200 mg of pure or enantiomerically enriched R-BDB, S-BDB, or racemic BDB, and a SSRI formulated for delayed release: 5-40 mg of vilazodone;
lx. Entactogen formulated for immediate release: 20-200 mg of pure or enantiomerically enriched R-BDB, S-BDB, or racemic BDB, and a SSRI formulated for delayed release: 2-15 mg of vortioxetine;
lxi. Entactogen formulated for immediate release: 20-200 mg of pure or enantiomerically enriched R-BDB, S-BDB, or racemic BDB, and a SNRI formulated for delayed release: 5-60 mg of duloxetine; and lxii. Entactogen formulated for immediate release: 20-200 mg of pure or enantiomerically enriched R-BDB, S-BDB, or racemic BDB, and a SNRI formulated for delayed release: 2-150 mg of milnacipran. lxiii. Entactogen formulated for immediate release: 20-200 mg of pure or enantiomerically enriched R-BDB, S-BDB, or racemic BDB, and a SNRI formulated for delayed release: 5-60 mg of levomilnacipran;
lxiv. Entactogen formulated for immediate release: 20-200 mg of pure or enantiomerically enriched R-BDB, S-BDB, or racemic BDB, and a SNRI formulated for delayed release: 5-200 mg of venlafaxine;
lxv. Entactogen formulated for immediate release: 20-200 mg of pure or enantiomerically enriched R-BDB, S-BDB, or racemic BDB, and a SNRI formulated for delayed release: 5-200 mg of desvenlafaxine;
lxvi. Entactogen formulated for immediate release: 10-200 mg of MDAI, and a SSRI formulated for delayed release: 2-20 mg of citalopram;
lxvii. Entactogen formulated for immediate release: 10-200 mg of MDAI, and a SSRI formulated for delayed release: 2-15 mg of escitalopram;
lxviii. Entactogen formulated for immediate release: 10-200 mg of MDAI, and a SSRI formulated for delayed release: 5-40 mg of fluoxetine;
lxix. Entactogen formulated for immediate release: 10-200 mg of MDAI, and a SSRI formulated for delayed release: 5-30 mg of paroxetine;
lxx. Entactogen formulated for immediate release: 10-200 mg of MDAI, and a SSRI formulated for delayed release: 10-150 mg of sertraline;
lxxi. Entactogen formulated for immediate release: 10-200 mg of MDAI, and a SSRI formulated for delayed release: 10-150 mg of fluvoxamine;
lxxii. Entactogen formulated for immediate release: 10-200 mg of MDAI, and a SSRI formulated for delayed release: 5-40 mg of vilazodone;
lxxiii. Entactogen formulated for immediate release: 10-200 mg of MDAI, and a SSRI formulated for delayed release: 2-15 mg of vortioxetine;
lxxiv. Entactogen formulated for immediate release: 10-200 mg of MDAI, and a SNRI formulated for delayed release: 5-60 mg of duloxetine;
lxxv. Entactogen formulated for immediate release: 10-200 mg of MDAI, and a SNRI formulated for delayed release: 2-150 mg of milnacipran;
lxxvi. Entactogen formulated for immediate release: 10-200 mg of MDAI, and a SNRI formulated for delayed release: 5-60 mg of levomilnacipran;
lxxvii. Entactogen formulated for immediate release: 10-200 mg of MDAI, and a SNRI formulated for delayed release: 5-200 mg of venlafaxine;
lxxviii. Entactogen formulated for immediate release: 10-200 mg of MDAI, and a SNRI formulated for delayed release: 5-200 mg of desvenlafaxine;
lxxix. Entactogen formulated for immediate release: 10-200 mg of MDMAI, and a SSRI formulated for delayed release: 2-20 mg of citalopram;
lxxx. Entactogen formulated for immediate release: 10-200 mg of MDMAI, and a SSRI formulated for delayed release: 2-15 mg of escitalopram;
lxxxi. Entactogen formulated for immediate release: 10-200 mg of MDMAI, and a SSRI formulated for delayed release: 5-40 mg of fluoxetine;
lxxxii. Entactogen formulated for immediate release: 10-200 mg of MDMAI, and a SSRI formulated for delayed release: 5-30 mg of paroxetine;
lxxxiii. Entactogen formulated for immediate release: 10-200 mg of MDMAI, and a SSRI formulated for delayed release: 10-150 mg of sertraline;
lxxxiv. Entactogen formulated for immediate release: 10-200 mg of MDMAI, and a SSRI formulated for delayed release: 10-150 mg of fluvoxamine;
lxxxv. Entactogen formulated for immediate release: 10-200 mg of MDMAI, and a SSRI formulated for delayed release: 5-40 mg of vilazodone;
lxxxvi. Entactogen formulated for immediate release: 10-200 mg of MDMAI, and a SSRI formulated for delayed release: 2-15 mg of vortioxetine;
lxxxvii. Entactogen formulated for immediate release: 10-200 mg of MDMAI, and a SNRI formulated for delayed release: 5-60 mg of duloxetine;
lxxxviii. Entactogen formulated for immediate release: 10-200 mg of MDMAI, and a SNRI formulated for delayed release: 2-150 mg of milnacipran;
lxxxix. Entactogen formulated for immediate release: 10-200 mg of MDMAI, and a SNRI formulated for delayed release: 5-60 mg of levomilnacipran;
xc. Entactogen formulated for immediate release: 10-200 mg of MDMAI, and a SNRI formulated for delayed release: 5-200 mg of venlafaxine;
xci. Entactogen formulated for immediate release: 10-200 mg of MDMAI, and a SNRI formulated for delayed release: 5-200 mg of desvenlafaxine;
xcii. Entactogen formulated for immediate release: 10-300 mg of pure or enantiomerically enriched R-MDAT, S-MDAT, or racemic MDAT, and a SSRI formulated for delayed release: 2-20 mg of citalopram;
xciii. Entactogen formulated for immediate release: 10-300 mg of pure or enantiomerically enriched R-MDAT, S-MDAT, or racemic MDAT, and a SSRI formulated for delayed release: 2-15 mg of escitalopram;
xciv. Entactogen formulated for immediate release: 10-300 mg of pure or enantiomerically enriched R-MDAT, S-MDAT, or racemic MDAT, and a SSRI formulated for delayed release: 5-40 mg of fluoxetine;
xcv. Entactogen formulated for immediate release: 10-300 mg of pure or enantiomerically enriched R-MDAT, S-MDAT, or racemic MDAT, and a SSRI formulated for delayed release: 5-30 mg of paroxetine;
xcvi. Entactogen formulated for immediate release: 10-300 mg of pure or enantiomerically enriched R-MDAT, S-MDAT, or racemic MDAT, and a SSRI formulated for delayed release: 10-150 mg of sertraline;
xcvii. Entactogen formulated for immediate release: 10-300 mg of pure or enantiomerically enriched R-MDAT, S-MDAT, or racemic MDAT, and a SSRI formulated for delayed release: 10-150 mg of fluvoxamine;
xcviii. Entactogen formulated for immediate release: 10-300 mg of pure or enantiomerically enriched R-MDAT, S-MDAT, or racemic MDAT, and a SSRI formulated for delayed release: 5-40 mg of vilazodone;
xcix. Entactogen formulated for immediate release: 10-300 mg of pure or enantiomerically enriched R-MDAT, S-MDAT, or racemic MDAT, and a SSRI formulated for delayed release: 2-15 mg of vortioxetine;
c. Entactogen formulated for immediate release: 10-300 mg of pure or enantiomerically enriched R-MDAT, S-MDAT, or racemic MDAT, and a SNRI formulated for delayed release: 5-60 mg of duloxetine; and
ci. Entactogen formulated for immediate release: 10-300 mg of pure or enantiomerically enriched R-MDAT, S-MDAT, or racemic MDAT, and a SNRI formulated for delayed release: 2-150 mg of milnacipran.
cii. Entactogen formulated for immediate release: 10-300 mg of pure or enantiomerically enriched R-MDAT, S-MDAT, or racemic MDAT, and a SNRI formulated for delayed release: 5-60 mg of levomilnacipran;
ciii. Entactogen formulated for immediate release: 10-300 mg of pure or enantiomerically enriched R-MDAT, S-MDAT, or racemic MDAT, and a SNRI formulated for delayed release: 5-200 mg of venlafaxine;
civ. Entactogen formulated for immediate release: 10-300 mg of pure or enantiomerically enriched R-MDAT, S-MDAT, or racemic MDAT, and a SNRI formulated for delayed release: 5-200 mg of desvenlafaxine;
cv. Entactogen formulated for immediate release: 10-300 mg of pure or enantiomerically enriched R-MDMAT, S-MDMAT, or racemic MDMAT, and a SSRI formulated for delayed release: 2-20 mg of citalopram;
cvi. Entactogen formulated for immediate release: 10-300 mg of pure or enantiomerically enriched R-MDMAT, S-MDMAT, or racemic MDMAT, and a SSRI formulated for delayed release: 2-15 mg of escitalopram;
cvii. Entactogen formulated for immediate release: 10-300 mg of pure or enantiomerically enriched R-MDMAT, S-MDMAT, or racemic MDMAT, and a SSRI formulated for delayed release: 5-40 mg of fluoxetine;
cviii. Entactogen formulated for immediate release: 10-300 mg of pure or enantiomerically enriched R-MDMAT, S-MDMAT, or racemic MDMAT, and a SSRI formulated for delayed release: 5-30 mg of paroxetine;
cix. Entactogen formulated for immediate release: 10-300 mg of pure or enantiomerically enriched R-MDMAT, S-MDMAT, or racemic MDMAT, and a SSRI formulated for delayed release: 10-150 mg of sertraline;
cx. Entactogen formulated for immediate release: 10-300 mg of pure or enantiomerically enriched R-MDMAT, S-MDMAT, or racemic MDMAT, and a SSRI formulated for delayed release: 10-150 mg of fluvoxamine;
cxi. Entactogen formulated for immediate release: 10-300 mg of pure or enantiomerically enriched R-MDMAT, S-MDMAT, or racemic MDMAT, and a SSRI formulated for delayed release: 5-40 mg of vilazodone;
cxii. Entactogen formulated for immediate release: 10-300 mg of pure or enantiomerically enriched R-MDMAT, S-MDMAT, or racemic MDMAT, and a SSRI formulated for delayed release: 2-15 mg of vortioxetine;
cxiii. Entactogen formulated for immediate release: 10-300 mg of pure or enantiomerically enriched R-MDMAT, S-MDMAT, or racemic MDMAT, and a SNRI formulated for delayed release: 5-60 mg of duloxetine; and
cxiv. Entactogen formulated for immediate release: 10-300 mg of pure or enantiomerically enriched R-MDMAT, S-MDMAT, or racemic MDMAT, and a SNRI formulated for delayed release: 2-150 mg of milnacipran.
cxv. Entactogen formulated for immediate release: 10-300 mg of pure or enantiomerically enriched R-MDMAT, S-MDMAT, or racemic MDMAT, and a SNRI formulated for delayed release: 5-60 mg of levomilnacipran;
cxvi. Entactogen formulated for immediate release: 10-300 mg of pure or enantiomerically enriched R-MDMAT, S-MDMAT, or racemic MDMAT, and a SNRI formulated for delayed release: 5-200 mg of venlafaxine;
cxvii. Entactogen formulated for immediate release: 10-300 mg of pure or enantiomerically enriched R-MDMAT, S-MDMAT, or racemic MDMAT, and a SNRI formulated for delayed release: 5-200 mg of desvenlafaxine;
cxviii. Entactogen formulated for immediate release: 20-250 mg of pure or enantiomerically enriched R-BK-MDMA, S-BK-MDMA, or racemic BK-MDMA, and a SSRI formulated for delayed release: 2-20 mg of citalopram;
cxix. Entactogen formulated for immediate release: 20-250 mg of pure or enantiomerically enriched R-BK-MDMA, S-BK-MDMA, or racemic BK-MDMA, and a SSRI formulated for delayed release: 2-15 mg of escitalopram;
cxx. Entactogen formulated for immediate release: 20-250 mg of pure or enantiomerically enriched R-BK-MDMA, S-BK-MDMA, or racemic BK-MDMA, and a SSRI formulated for delayed release: 5-40 mg of fluoxetine;
cxxi. Entactogen formulated for immediate release: 20-250 mg of pure or enantiomerically enriched R-BK-MDMA, S-BK-MDMA, or racemic BK-MDMA, and a SSRI formulated for delayed release: 5-30 mg of paroxetine;
cxxii. Entactogen formulated for immediate release: 20-250 mg of pure or enantiomerically enriched R-BK-MDMA, S-BK-MDMA, or racemic BK-MDMA, and a SSRI formulated for delayed release: 10-150 mg of sertraline;
cxxiii. Entactogen formulated for immediate release: 20-250 mg of pure or enantiomerically enriched R-BK-MDMA, S-BK-MDMA, or racemic BK-MDMA, and a SSRI formulated for delayed release: 10-150 mg of fluvoxamine;
cxxiv. Entactogen formulated for immediate release: 20-250 mg of pure or enantiomerically enriched R-BK-MDMA, S-BK-MDMA, or racemic BK-MDMA, and a SSRI formulated for delayed release: 5-40 mg of vilazodone;
cxxv. Entactogen formulated for immediate release: 20-250 mg of pure or enantiomerically enriched R-BK-MDMA, S-BK-MDMA, or racemic BK-MDMA, and a SSRI formulated for delayed release: 2-15 mg of vortioxetine;
cxxvi. Entactogen formulated for immediate release: 20-250 mg of pure or enantiomerically enriched R-BK-MDMA, S-BK-MDMA, or racemic BK-MDMA, and a SNRI formulated for delayed release: 5-60 mg of duloxetine; and
cxxvii. Entactogen formulated for immediate release: 20-250 mg of pure or enantiomerically enriched R-BK-MDMA, S-BK-MDMA, or racemic BK-MDMA, and a SNRI formulated for delayed release: 2-150 mg of milnacipran.
cxxviii. Entactogen formulated for immediate release: 20-250 mg of pure or enantiomerically enriched R-BK-MDMA, S-BK-MDMA, or racemic BK-MDMA, and a SNRI formulated for delayed release: 5-60 mg of levomilnacipran;
cxxix. Entactogen formulated for immediate release: 20-250 mg of pure or enantiomerically enriched R-BK-MDMA, S-BK-MDMA, or racemic BK-MDMA, and a SNRI formulated for delayed release: 5-200 mg of venlafaxine;
cxxx. Entactogen formulated for immediate release: 20-250 mg of pure or enantiomerically enriched R-BK-MDMA, S-BK-MDMA, or racemic BK-MDMA, and a SNRI formulated for delayed release: 5-200 mg of desvenlafaxine;
cxxxi. Entactogen formulated for immediate release: 20-250 mg of pure or enantiomerically enriched R-BK-MDEA, S-BK-MDEA, or racemic BK-MDEA, and a SSRI formulated for delayed release: 2-20 mg of citalopram;
cxxxii. Entactogen formulated for immediate release: 20-250 mg of pure or enantiomerically enriched R-BK-MDEA, S-BK-MDEA, or racemic BK-MDEA, and a SSRI formulated for delayed release: 2-15 mg of escitalopram;
cxxxiii. Entactogen formulated for immediate release: 20-250 mg of pure or enantiomerically enriched R-BK-MDEA, S-BK-MDEA, or racemic BK-MDEA, and a SSRI formulated for delayed release: 5-40 mg of fluoxetine;
cxxxiv. Entactogen formulated for immediate release: 20-250 mg of pure or enantiomerically enriched R-BK-MDEA, S-BK-MDEA, or racemic BK-MDEA, and a SSRI formulated for delayed release: 5-30 mg of paroxetine;
cxxxv. Entactogen formulated for immediate release: 20-250 mg of pure or enantiomerically enriched R-BK-MDEA, S-BK-MDEA, or racemic BK-MDEA, and a SSRI formulated for delayed release: 10-150 mg of sertraline;
cxxxvi. Entactogen formulated for immediate release: 20-250 mg of pure or enantiomerically enriched R-BK-MDEA, S-BK-MDEA, or racemic BK-MDEA, and a SSRI formulated for delayed release: 10-150 mg of fluvoxamine;
cxxxvii. Entactogen formulated for immediate release: 20-250 mg of pure or enantiomerically enriched R-BK-MDEA, S-BK-MDEA, or racemic BK-MDEA, and a SSRI formulated for delayed release: 5-40 mg of vilazodone;
cxxxviii. Entactogen formulated for immediate release: 20-250 mg of pure or enantiomerically enriched R-BK-MDEA, S-BK-MDEA, or racemic BK-MDEA, and a SSRI formulated for delayed release: 2-15 mg of vortioxetine;
cxxxix. Entactogen formulated for immediate release: 20-250 mg of pure or enantiomerically enriched R-BK-MDEA, S-BK-MDEA, or racemic BK-MDEA, and a SNRI formulated for delayed release: 5-60 mg of duloxetine; and
cxl. Entactogen formulated for immediate release: 20-250 mg of pure or enantiomerically enriched R-BK-MDEA, S-BK-MDEA, or racemic BK-MDEA, and a SNRI formulated for delayed release: 2-150 mg of milnacipran.
cxli. Entactogen formulated for immediate release: 20-250 mg of pure or enantiomerically enriched R-BK-MDEA, S-BK-MDEA, or racemic BK-MDEA, and a SNRI formulated for delayed release: 5-60 mg of levomilnacipran;
cxlii. Entactogen formulated for immediate release: 20-250 mg of pure or enantiomerically enriched R-BK-MDEA, S-BK-MDEA, or racemic BK-MDEA, and a SNRI formulated for delayed release: 5-200 mg of venlafaxine;
cxliii. Entactogen formulated for immediate release: 20-250 mg of pure or enantiomerically enriched R-BK-MDEA, S-BK-MDEA, or racemic BK-MDEA, and a SNRI formulated for delayed release: 5-200 mg of desvenlafaxine;
cxliv. Entactogen formulated for immediate release: 5-100 mg of pure or enantiomerically enriched R-5-APB, S-5-APB, or racemic 5-APB, and a SSRI formulated for delayed release: 2-20 mg of citalopram;
cxlv. Entactogen formulated for immediate release: 5-100 mg of pure or enantiomerically enriched R-5-APB, S-5-APB, or racemic 5-APB, and a SSRI formulated for delayed release: 2-15 mg of escitalopram;
cxlvi. Entactogen formulated for immediate release: 5-150 mg of pure or enantiomerically enriched R-5-APB, S-5-APB, or racemic 5-APB, and a SSRI formulated for delayed release: 5-40 mg of fluoxetine;
cxlvii. Entactogen formulated for immediate release: 5-100 mg of pure or enantiomerically enriched R-5-APB, S-5-APB, or racemic 5-APB, and a SSRI formulated for delayed release: 5-30 mg of paroxetine;
cxlviii. Entactogen formulated for immediate release: 5-100 mg of pure or enantiomerically enriched R-5-APB, S-5-APB, or racemic 5-APB, and a SSRI formulated for delayed release: 10-150 mg of sertraline;
cxlix. Entactogen formulated for immediate release: 5-100 mg of pure or enantiomerically enriched R-5-APB, S-5-APB, or racemic 5-APB, and a SSRI formulated for delayed release: 10-150 mg of fluvoxamine;
cl. Entactogen formulated for immediate release: 5-150 mg of pure or enantiomerically enriched R-5-APB, S-5-APB, or racemic 5-APB, and a SSRI formulated for delayed release: 5-40 mg of vilazodone;
cli. Entactogen formulated for immediate release: 5-100 mg of pure or enantiomerically enriched R-5-APB, S-5-APB, or racemic 5-APB, and a SSRI formulated for delayed release: 5-30 mg of vortioxetine;
clii. Entactogen formulated for immediate release: 5-100 mg of pure or enantiomerically enriched R-5-APB, S-5-APB, or racemic 5-APB, and a SNRI formulated for delayed release: 5-60 mg of duloxetine; and
cliii. Entactogen formulated for immediate release: 5-100 mg of pure or enantiomerically enriched R-5-APB, S-5-APB, or racemic 5-APB, and a SNRI formulated for delayed release: 2-150 mg of milnacipran.
cliv. Entactogen formulated for immediate release: 5-100 mg of pure or enantiomerically enriched R-5-APB, S-5-APB, or racemic 5-APB, and a SNRI formulated for delayed release: 5-60 mg of levomilnacipran;
clv. Entactogen formulated for immediate release: 5-100 mg of pure or enantiomerically enriched R-5-APB, S-5-APB, or racemic 5-APB, and a SNRI formulated for delayed release: 5-200 mg of venlafaxine;
clvi. Entactogen formulated for immediate release: 5-100 mg of pure or enantiomerically enriched R-5-APB, S-5-APB, or racemic 5-APB, and a SNRI formulated for delayed release: 5-200 mg of desvenlafaxine;
clvii. Entactogen formulated for immediate release: 5-100 mg of pure or enantiomerically enriched R-6-APB, S-6-APB, or racemic 6-APB, and a SSRI formulated for delayed release: 2-20 mg of citalopram;
clviii. Entactogen formulated for immediate release: 5-100 mg of pure or enantiomerically enriched R-6-APB, S-6-APB, or racemic 6-APB, and a SSRI formulated for delayed release: 2-15 mg of escitalopram;
clix. Entactogen formulated for immediate release: 5-100 mg of pure or enantiomerically enriched R-6-APB, S-6-APB, or racemic 6-APB, and a SSRI formulated for delayed release: 5-40 mg of fluoxetine;
clx. Entactogen formulated for immediate release: 5-100 mg of pure or enantiomerically enriched R-6-APB, S-6-APB, or racemic 6-APB, and a SSRI formulated for delayed release: 5-30 mg of paroxetine;
clxi. Entactogen formulated for immediate release: 5-100 mg of pure or enantiomerically enriched R-6-APB, S-6-APB, or racemic 6-APB, and a SSRI formulated for delayed release: 10-150 mg of sertraline;
clxii. Entactogen formulated for immediate release: 5-100 mg of pure or enantiomerically enriched R-6-APB, S-6-APB, or racemic 6-APB, and a SSRI formulated for delayed release: 10-150 mg of fluvoxamine;
clxiii. Entactogen formulated for immediate release: 5-100 mg of pure or enantiomerically enriched R-6-APB, S-6-APB, or racemic 6-APB, and a SSRI formulated for delayed release: 5-40 mg of vilazodone;
clxiv. Entactogen formulated for immediate release: 5-100 mg of pure or enantiomerically enriched R-6-APB, S-6-APB, or racemic 6-APB, and a SSRI formulated for delayed release: 2-15 mg of vortioxetine;
clxv. Entactogen formulated for immediate release: 5-100 mg of pure or enantiomerically enriched R-6-APB, S-6-APB, or racemic 6-APB, and a SNRI formulated for delayed release: 5-60 mg of duloxetine; and
clxvi. Entactogen formulated for immediate release: 5-100 mg of pure or enantiomerically enriched R-6-APB, S-6-APB, or racemic 6-APB, and a SNRI formulated for delayed release: 2-150 mg of milnacipran.
clxvii. Entactogen formulated for immediate release: 5-100 mg of pure or enantiomerically enriched R-6-APB, S-6-APB, or racemic 6-APB, and a SNRI formulated for delayed release: 5-60 mg of levomilnacipran;
clxviii. Entactogen formulated for immediate release: 5-100 mg of pure or enantiomerically enriched R-6-APB, S-6-APB, or racemic 6-APB, and a SNRI formulated for delayed release: 5-200 mg of venlafaxine;
clxix. Entactogen formulated for immediate release: 5-150 mg of pure or enantiomerically enriched R-6-APB, S-6-APB, or racemic 6-APB, and a SNRI formulated for delayed release: 5-200 mg of desvenlafaxine;
clxx. Entactogen formulated for immediate release: 5-100 mg of pure or enantiomerically enriched R-5-MAPB, S-5-MAPB, or racemic 5-MAPB, and a SSRI formulated for delayed release: 2-20 mg of citalopram;
clxxi. Entactogen formulated for immediate release: 5-100 mg of pure or enantiomerically enriched R-5-MAPB, S-5-MAPB, or racemic 5-MAPB, and a SSRI formulated for delayed release: 2-15 mg of escitalopram;
clxxii. Entactogen formulated for immediate release: 5-100 mg of pure or enantiomerically enriched R-5-MAPB, S-5-MAPB, or racemic 5-MAPB, and a SSRI formulated for delayed release: 5-40 mg of fluoxetine;
clxxiii. Entactogen formulated for immediate release: 5-100 mg of pure or enantiomerically enriched R-5-MAPB, S-5-MAPB, or racemic 5-MAPB, and a SSRI formulated for delayed release: 5-30 mg of paroxetine;
clxxiv. Entactogen formulated for immediate release: 5-100 mg of pure or enantiomerically enriched R-5-MAPB, S-5-MAPB, or racemic 5-MAPB, and a SSRI formulated for delayed release: 10-150 mg of sertraline;
clxxv. Entactogen formulated for immediate release: 5-100 mg of pure or enantiomerically enriched R-5-MAPB, S-5-MAPB, or racemic 5-MAPB, and a SSRI formulated for delayed release: 10-150 mg of fluvoxamine;
clxxvi. Entactogen formulated for immediate release: 5-100 mg of pure or enantiomerically enriched R-5-MAPB, S-5-MAPB, or racemic 5-MAPB, and a SSRI formulated for delayed release: 5-40 mg of vilazodone;
clxxvii. Entactogen formulated for immediate release: 5-100 mg of pure or enantiomerically enriched R-5-MAPB, S-5-MAPB, or racemic 5-MAPB, and a SSRI formulated for delayed release: 2-15 mg of vortioxetine;
clxxviii. Entactogen formulated for immediate release: 5-100 mg of pure or enantiomerically enriched R-5-MAPB, S-5-MAPB, or racemic 5-MAPB, and a SNRI formulated for delayed release: 5-60 mg of duloxetine; and
clxxix. Entactogen formulated for immediate release: 5-100 mg of pure or enantiomerically enriched R-5-MAPB, S-5-MAPB, or racemic 5-MAPB, and a SNRI formulated for delayed release: 2-150 mg of milnacipran.
clxxx. Entactogen formulated for immediate release: 5-100 mg of pure or enantiomerically enriched R-5-MAPB, S-5-MAPB, or racemic 5-MAPB, and a SNRI formulated for delayed release: 5-60 mg of levomilnacipran;
clxxxi. Entactogen formulated for immediate release: 5-100 mg of pure or enantiomerically enriched R-5-MAPB, S-5-MAPB, or racemic 5-MAPB, and a SNRI formulated for delayed release: 5-200 mg of venlafaxine;
clxxxii. Entactogen formulated for immediate release: 5-100 mg of pure or enantiomerically enriched R-5-MAPB, S-5-MAPB, or racemic 5-MAPB, and a SNRI formulated for delayed release: 5-200 mg of desvenlafaxine;
clxxxiii. Entactogen formulated for immediate release: 5-100 mg of pure or enantiomerically enriched R-6-MAPB, S-6-MAPB, or racemic 6-MAPB, and a SSRI formulated for delayed release: 2-20 mg of citalopram;
clxxxiv. Entactogen formulated for immediate release: 5-100 mg of pure or enantiomerically enriched R-6-MAPB, S-6-MAPB, or racemic 6-MAPB, and a SSRI formulated for delayed release: 2-15 mg of escitalopram;
clxxxv. Entactogen formulated for immediate release: 5-100 mg of pure or enantiomerically enriched R-6-MAPB, S-6-MAPB, or racemic 6-MAPB, and a SSRI formulated for delayed release: 5-40 mg of fluoxetine;
clxxxvi. Entactogen formulated for immediate release: 5-100 mg of pure or enantiomerically enriched R-6-MAPB, S-6-MAPB, or racemic 6-MAPB, and a SSRI formulated for delayed release: 5-30 mg of paroxetine;
clxxxvii. Entactogen formulated for immediate release: 5-100 mg of pure or enantiomerically enriched R-6-MAPB, S-6-MAPB, or racemic 6-MAPB, and a SSRI formulated for delayed release: 10-150 mg of sertraline;
clxxxviii. Entactogen formulated for immediate release: 5-100 mg of pure or enantiomerically enriched R-6-MAPB, S-6-MAPB, or racemic 6-MAPB, and a SSRI formulated for delayed release: 10-150 mg of fluvoxamine;
clxxxix. Entactogen formulated for immediate release: 5-100 mg of pure or enantiomerically enriched R-6-MAPB, S-6-MAPB, or racemic 6-MAPB, and a SSRI formulated for delayed release: 5-40 mg of vilazodone;
cxc. Entactogen formulated for immediate release: 5-100 mg of pure or enantiomerically enriched R-6-MAPB, S-6-MAPB, or racemic 6-MAPB, and a SSRI formulated for delayed release: 2-15 mg of vortioxetine;
cxci. Entactogen formulated for immediate release: 5-100 mg of pure or enantiomerically enriched R-6-MAPB, S-6-MAPB, or racemic 6-MAPB, and a SNRI formulated for delayed release: 5-60 mg of duloxetine; and
cxcii. Entactogen formulated for immediate release: 5-100 mg of pure or enantiomerically enriched R-6-MAPB, S-6-MAPB, or racemic 6-MAPB, and a SNRI formulated for delayed release: 2-150 mg of milnacipran.
cxciii. Entactogen formulated for immediate release: 5-100 mg of pure or enantiomerically enriched R-6-MAPB, S-6-MAPB, or racemic 6-MAPB, and a SNRI formulated for delayed release: 5-60 mg of levomilnacipran;
cxciv. Entactogen formulated for immediate release: 5-100 mg of pure or enantiomerically enriched R-6-MAPB, S-6-MAPB, or racemic 6-MAPB, and a SNRI formulated for delayed release: 5-200 mg of venlafaxine; and
cxcv. Entactogen formulated for immediate release: 5-100 mg of pure or enantiomerically enriched R-6-MAPB, S-6-MAPB, or racemic 6-MAPB, and a SNRI formulated for delayed release: 5-200 mg of desvenlafaxine.
cxcvi. Entactogen formulated for immediate release: 2-100 mg of pure or enantiomerically enriched R-BK-5-MAPB, S-BK-5-MAPB, or racemic BK-5-MAPB, and a SSRI formulated for delayed release: 2-20 mg of citalopram;
cxcvii. Entactogen formulated for immediate release: 2-100 mg of pure or enantiomerically enriched R-BK-5-MAPB, S-BK-5-MAPB, or racemic BK-5-MAPB, and a SSRI formulated for delayed release: 2-15 mg of escitalopram;
cxcviii. Entactogen formulated for immediate release: 2-100 mg of pure or enantiomerically enriched R-BK-5-MAPB, S-BK-5-MAPB, or racemic BK-5-MAPB, and a SSRI formulated for delayed release: 5-40 mg of fluoxetine;
cxcix. Entactogen formulated for immediate release: 2-100 mg of pure or enantiomerically enriched R-BK-5-MAPB, S-BK-5-MAPB, or racemic BK-5-MAPB, and a SSRI formulated for delayed release: 5-30 mg of paroxetine;
cc. Entactogen formulated for immediate release: 2-100 mg of pure or enantiomerically enriched R-BK-5-MAPB, S-BK-5-MAPB, or racemic BK-5-MAPB, and a SSRI formulated for delayed release: 10-150 mg of sertraline;
cci. Entactogen formulated for immediate release: 2-100 mg of pure or enantiomerically enriched R-BK-5-MAPB, S-BK-5-MAPB, or racemic BK-5-MAPB, and a SSRI formulated for delayed release: 10-150 mg of fluvoxamine;
ccii. Entactogen formulated for immediate release: 2-100 mg of pure or enantiomerically enriched R-BK-5-MAPB, S-BK-5-MAPB, or racemic BK-5-MAPB, and a SSRI formulated for delayed release: 5-40 mg of vilazodone;
cciii. Entactogen formulated for immediate release: 2-100 mg of pure or enantiomerically enriched R-BK-5-MAPB, S-BK-5-MAPB, or racemic BK-5-MAPB, and a SSRI formulated for delayed release: 2-15 mg of vortioxetine;
cciv. Entactogen formulated for immediate release: 2-100 mg of pure or enantiomerically enriched R-BK-5-MAPB, S-BK-5-MAPB, or racemic BK-5-MAPB, and a SNRI formulated for delayed release: 5-60 mg of duloxetine; and
ccv. Entactogen formulated for immediate release: 2-100 mg of pure or enantiomerically enriched R-BK-5-MAPB, S-BK-5-MAPB, or racemic BK-5-MAPB, and a SNRI formulated for delayed release: 2-150 mg of milnacipran.
ccvi. Entactogen formulated for immediate release: 2-100 mg of pure or enantiomerically enriched R-BK-5-MAPB, S-BK-5-MAPB, or racemic BK-5-MAPB, and a SNRI formulated for delayed release: 5-60 mg of levomilnacipran;
ccvii. Entactogen formulated for immediate release: 2-100 mg of pure or enantiomerically enriched R-BK-5-MAPB, S-BK-5-MAPB, or racemic BK-5-MAPB, and a SNRI formulated for delayed release: 5-200 mg of venlafaxine; and
ccviii. Entactogen formulated for immediate release: 2-100 mg of pure or enantiomerically enriched R-BK-5-MAPB, S-BK-5-MAPB, or racemic BK-5-MAPB, and a SNRI formulated for delayed release: 5-200 mg of desvenlafaxine.
ccix. Entactogen formulated for immediate release: 2-100 mg of pure or enantiomerically enriched R-BK-5-EAPB, S-BK-5-EAPB, or racemic BK-5-EAPB, and a SSRI formulated for delayed release: 2-20 mg of citalopram;
ccx. Entactogen formulated for immediate release: 2-100 mg of pure or enantiomerically enriched R-BK-5-EAPB, S-BK-5-EAPB, or racemic BK-5-EAPB, and a SSRI formulated for delayed release: 2-15 mg of escitalopram;
ccxi. Entactogen formulated for immediate release: 2-100 mg of pure or enantiomerically enriched R-BK-5-EAPB, S-BK-5-EAPB, or racemic BK-5-EAPB, and a SSRI formulated for delayed release: 5-40 mg of fluoxetine;
ccxii. Entactogen formulated for immediate release: 2-100 mg of pure or enantiomerically enriched R-BK-5-EAPB, S-BK-5-EAPB, or racemic BK-5-EAPB, and a SSRI formulated for delayed release: 5-30 mg of paroxetine;
ccxiii. Entactogen formulated for immediate release: 2-100 mg of pure or enantiomerically enriched R-BK-5-EAPB, S-BK-5-EAPB, or racemic BK-5-EAPB, and a SSRI formulated for delayed release: 10-150 mg of sertraline;
ccxiv. Entactogen formulated for immediate release: 2-100 mg of pure or enantiomerically enriched R-BK-5-EAPB, S-BK-5-EAPB, or racemic BK-5-EAPB, and a SSRI formulated for delayed release: 10-150 mg of fluvoxamine;
ccxv. Entactogen formulated for immediate release: 2-100 mg of pure or enantiomerically enriched R-BK-5-EAPB, S-BK-5-EAPB, or racemic BK-5-EAPB, and a SSRI formulated for delayed release: 5-40 mg of vilazodone;
ccxvi. Entactogen formulated for immediate release: 2-100 mg of pure or enantiomerically enriched R-BK-5-EAPB, S-BK-5-EAPB, or racemic BK-5-EAPB, and a SSRI formulated for delayed release: 2-15 mg of vortioxetine;
ccxvii. Entactogen formulated for immediate release: 5-200 mg of pure or enantiomerically enriched R-BK-5-EAPB, S-BK-5-EAPB, or racemic BK-5-EAPB, and a SNRI formulated for delayed release: 5-60 mg of duloxetine; and
ccxviii. Entactogen formulated for immediate release: 2-100 mg of pure or enantiomerically enriched R-BK-5-EAPB, S-BK-5-EAPB, or racemic BK-5-EAPB, and a SNRI formulated for delayed release: 2-150 mg of milnacipran.
ccxix. Entactogen formulated for immediate release: 2-100 mg of pure or enantiomerically enriched R-BK-5-EAPB, S-BK-5-EAPB, or racemic BK-5-EAPB, and a SNRI formulated for delayed release: 5-60 mg of levomilnacipran;
ccxx. Entactogen formulated for immediate release: 2-100 mg of pure or enantiomerically enriched R-BK-5-EAPB, S-BK-5-EAPB, or racemic BK-5-EAPB, and a SNRI formulated for delayed release: 5-200 mg of venlafaxine; and
ccxxi. Entactogen formulated for immediate release: 5-200 mg of pure or enantiomerically enriched R-BK-5-EAPB, S-BK-5-EAPB, or racemic BK-5-EAPB, and a SNRI formulated for delayed release: 5-200 mg of desvenlafaxine.

### Exemplary Disorders

A pharmaceutical composition of the present invention can be used in methods for treating a variety of diseases or disorders linked to inadequate functioning of neurotransmission in the CNS of mammals. Included among such disorders are depression, dysthymia, anxiety and phobia disorders (including generalized anxiety, social anxiety, panic, post-traumatic stress and adjustment disorders), feeding and eating disorders (including binge eating, bulimia, and anorexia nervosa), other binge behaviors, body dysmorphic syndromes, alcoholism, tobacco abuse, drug abuse or dependence disorders, disruptive behavior disorders, impulse control disorders, gaming disorders, gambling disorders, memory loss, dementia of aging, attention deficit hyperactivity disorder, personality disorders (including antisocial, avoidant, borderline, histrionic, narcissistic, obsessive compulsive, paranoid, schizoid and schizotypal personality disorders), attachment disorders, autism, and dissociative disorders. Also included among such disorders are primary or secondary headaches. Also included among such disorders are seizure disorders, such as epilepsy disorders.

In addition to treating various diseases and disorders, the employed methods of modulating activity of the serotonergic system in particular can be used to improve CNS functioning in non-disease states, such as reducing neuroticism and psychological defensiveness, increasing openness to experience, increasing creativity, and aiding decision-making. These methods can employ a pharmaceutical composition of the present invention comprising an entactogen either as a racemate, an individual enantiomer, an enantiomerically enriched mixture, or with deuterium-substitution, or more than one of these in combination. When referring to entactogens for use in combinations herein, the terms accordingly should be understood to refer not only to the racemates of those structures, but also to single enantiomers, enantiomerically enriched mixtures, and structures with deuterium-substitution(s) or other modifications, as the context indicates and supports.

In certain aspects, the entactogenic properties of certain combinations can be improved by administering an enantiomerically enriched entactogen that has an abundance of one enantiomer over the other, or for some of the entactogens described herein, a substantially pure enantiomer (or diastereomer, where relevant). It has been discovered that certain entactogens in enantiomerically enriched form act differently from the racemate on various 5-HT receptors, dopamine receptors, nicotinic acetylcholine receptors, and norepinephrine receptors, producing variable effects, and that those effects can be selected for based on desired outcome for the patient. This could not be predicted in advance given the complexity of the neurotransmitter system.

This invention also provides the use of a pharmaceutical composition for the manufacture of a medicament for the treatment of maladaptive response to perceived psychological threats. Additionally, this invention provides a pharmaceutical formulation adapted for the treatment of maladaptive response to perceived psychological threats containing a combination described herein; or a pharmaceutically acceptable salt or salt mixture thereof. Furthermore, this invention includes a method for the treatment of maladaptive response to perceived psychological threats that comprises administering an effective amount of a pharmaceutical composition of the present invention, given either in the context of psychotherapy or as a stand-alone treatment.

In certain aspects an entactogen for use in a pharmaceutical composition of the present invention has improved biological properties compared to MDMA and can be used to advantageously treat a disorder described herein. For example, in certain embodiments an entactogen in a pharmaceutical composition of the present invention has decreased formation of phase I or phase II metabolites. This, in turn, can lead to less formation of reactive oxygen species or other forms of metabolites and oxidative stress. In certain aspects the pharmaceutical composition of the present invention causes less acute or lasting decreases in markers of monoaminergic neuron functioning, such as decreased serotonin or serotonin transporter than MDMA alone. In certain embodiments pharmaceutical composition of the present invention has a slower or smaller tolerance formation compared to MDMA alone or fewer side effects compared to MDMA alone.

### Methods to Treat Headache Disorders

In certain embodiments, a method of treating a patient with primary or secondary headaches is provided, comprising administering an effective amount of a pharmaceutical composition or combination of the present invention. While administration of such a composition typically occurs as needed (i.e., at the onset of headache or prodromal syndrome), in some cases, administration may be monthly, weekly, daily, twice daily, or a similar interval to achieve adequate symptom relief. Because some headache disorders have cyclical or other patterns to their occurrence, it is contemplated that medication could be taken using a personalized schedule that is based on use of an algorithm to predict the onset of headache. Administration may be oral, but other routes including buccal, sublingual, inhaled, and other parenteral routes are contemplated. In some embodiments, routes with fast onset of therapeutic effects are considered advantageous.

As used herein, primary headaches include, but are not limited to migraine, migraine signs and symptoms without cephalgia, tension-type headaches, cluster headaches and other trigeminal autonomic cephalalgias, new daily persistent headache, hypnic headaches, stabbing headaches, and other primary headache disorders. Secondary headaches referred to herein can refer to those due to trauma or injury, cranial or cervical vascular disorder, non-vascular intracranial disorder, headaches due to substance use or substance withdrawal, and other secondary headaches.

In certain embodiments a pharmaceutical composition of the present invention is used to treat a migraine, headache, or cluster headache. Non-limiting examples of migraines include migraine without aura, migraine with aura, chronic migraine, abdominal migraine, acephalgic migraine, silent migraine, migraine with brainstem aura, hemiplegic migraine, retinal migraine, and status migrainosus.

Improvement in headache disorders is typically indicated by improvements (lessening of severity or frequency) in pain, nausea, photophobia, and phonophobia and the accompanying disruption of normal activities (Loder and Burch 2012. Cephalalgia, 32(3), pp. 179-182; Vingen et al. 1998. Cephalalgia, 18(5), pp. 250-256; Sauro et al. 2010. Headache: The Journal of Head and Face Pain, 50(3), pp. 383-395). As such, it is anticipated that the methods of treatment disclosed within will result in improvements of one or more of these signs and symptoms.

### Methods to Treat Seizure Disorders

In certain embodiments, a method of treating a patient with a seizure disorder is provided, comprising administering an effective amount of a pharmaceutical composition or combination described herein. While administration of such a composition typically occurs daily or twice daily, in some cases, administration may be as needed (i.e., at the onset of a prodromal syndrome). Because some seizure disorders have cyclical or other patterns to their occurrence, it is contemplated that medication could be taken using a personalized schedule that is based on use of an algorithm to predict the onset of seizures. Administration may be oral, but other routes including buccal, sublingual, inhaled, and other parenteral routes are contemplated. In some embodiments, routes with fast onset of therapeutic effects are considered advantageous.

As used herein, seizure disorders include, but are not limited to focal aware seizures, focal impaired awareness seizures, bilateral tonic-clonic seizures, absence seizures, atyptical absence seizures, tonic-clonic seizures, atonic seizures, clonic seizures, tonic seizures, myoclonic seizures, gelastic seizures, and dacrystic seizures. In certain embodiments a pharmaceutical composition is used to treat epilepsy. In certain embodiments, the form of epilepsy is severe myoclonic epilepsy of infancy.

Improvement in seizure disorders is understood to mean a decrease in frequency or severity (or both frequency and severity), which can be assessed with both self-report and objective measures (e.g., EEG, including wearable devices) (Cramer and French 2001. Epilepsia, 42(1), pp. 119-129; Karoly, Goldenholz, and Cook 2018. Current opinion in neurology, 31(2), pp. 162-168). As such, it is anticipated that the methods of treatment disclosed within will result in improvements of one or more of these signs and symptoms.

### Methods to Treat Alcohol Use Disorders

In certain embodiments, a method of treating a patient with an alcohol use disorder is provided, comprising administering an effective amount of a pharmaceutical composition or combination described herein. In certain embodiments administration of the composition occurs daily or twice daily. In other embodiments administration may be as needed.

As used herein, alcohol use disorders include, but are not limited to alcohol abuse, alcohol dependence, and alcohol addiction. In certain embodiments a pharmaceutical composition is used to treat mild alcohol use disorders. In certain embodiments a pharmaceutical composition is used to treat moderate alcohol use disorders. In certain embodiments a pharmaceutical composition is used to treat severe alcohol use disorders.

### Non-Limiting Examples of Pharmacotherapeutic Counseling Use

In certain aspects psychotherapy, cognitive enhancement, or life coaching conducted with a pharmaceutical composition or combination described herein is employed as an adjunct (hereafter, “pharmacotherapy” or “pharmacotherapy counseling”) is typically conducted in widely spaced sessions with one, two, or rarely three or more administrations of an entactogen per session. These sessions can be as frequent as weekly but are more often approximately monthly or even less frequently. In most cases, a small number of pharmacotherapy counseling sessions, on the order of one to three, is needed for the patient to experience significant clinical progress, as indicated, for example, by a reduction in signs and symptoms of mental distress, by improvement in functioning in some domain of life, by arrival at a satisfactory solution to some problem, or by increased feelings of closeness to and understanding of some other person. In some embodiments, the psychotherapy, cognitive enhancement, or life coaching is conducted with an effective amount of a pharmaceutical composition, wherein the entactogen is present in an enantiomerically enriched mixture or a pharmaceutically acceptable salt thereof.

Use of a Composition or Combination of the Present Invention in Conjunction with Conventional Psychotherapy or Coaching

In certain embodiments, the use of a composition or combination of the present invention as pharmacotherapy is integrated into the patient's ongoing psychotherapy or coaching (hereafter abbreviated as “psychotherapy”). If a patient in need of the pharmacotherapy is not in ongoing psychotherapy, then psychotherapy may be initiated and the pharmacotherapy added later, after the prescribing physician and treating psychotherapist, physician, coach, member of the clergy, or other similar professional or someone acting under the supervision of such a professional (hereafter, “therapist”) agree that the pharmacotherapy is indicated and that there have been sufficient meetings between the patient and therapist to establish an effective therapeutic alliance.

If the patient is not experienced with the pharmacotherapy, a conversation typically occurs in which the therapist or other members of the therapy team addresses the patient's questions and concerns about the medicine and familiarizes the patient with the logistics of pharmacotherapy-assisted session. The therapist describes the kinds of experience that can be expected during the pharmacotherapy session. Optionally, parts of this conversation employ written, recorded, or interactive digital explanations, as might be used in the informed consent process in a clinical trial. The therapist may additionally make commitments to support the participant's healthcare and wellness process. In turn, the patient may be asked to make commitments of their own (such as not to hurt themselves or others and to abstain from contraindicated medicines or drugs for an adequate period before and after the pharmacotherapy).

The composition or combination of the invention (or alternately herein for convenience, the “medicine”) is administered shortly before or during a scheduled psychotherapy session, with timing optionally selected so that therapeutic effects begin by the time the psychotherapy session begins. Either shortly before or after administration of the medicine, it is common for the therapist to provide some reminder of their mutual commitments and expected events during the session.

The psychotherapy session is carried out by the therapist, who, optionally, may be remote and in communication with the patient using a communication means suitable for telehealth or telemedicine, such as a phone, video, or other remote two-way communication method. Optionally, video or other monitoring of the patient's response or behavior is used to document or measure the session. The therapist uses their clinical judgment and available data to adjust the session to the needs of the patient. Many therapists view their responsibility as being to facilitate rather than direct the patient's experience. This may sometimes involve silent empathic listening, while other times it may include more active support to help the patient arrive at new perspectives on their life.

It is anticipated that the therapeutic effects of the medicine will allow the patient to make more rapid therapeutic progress than would normally be possible. These effects include decreased neuroticism and increased feelings of authenticity. Patients are often able to calmly contemplate actual or possible experiences that would normally be upsetting or even overwhelming. This can facilitate decision making and creativity in addition to mental wellness.

Optionally, the prescribing physician may allow a second or even third administration of the medicine or another psychotherapeutic agent in order to extend the therapeutic effects. Optionally, a pharmaceutical preparation with modified release is employed to make this unnecessary.

Because the duration of the scheduled psychotherapy session may be shorter than the therapeutic effects of the medicine, the therapist may suggest to the patient activities to support further psychotherapeutic progress after the psychotherapy session has ended. Alternatively, the therapist may continue to work with the patient until the therapeutic effects of the medicine have become clinically minimal.

In a subsequent non-pharmacological psychotherapy session, the therapist and patient will typically discuss the patient's experiences from the pharmacotherapy session and the therapist will often aid the patient in recalling the therapeutic effects and help them to incorporate the experiences into their everyday lives.

Pharmacotherapy sessions may be repeated as needed, based on the judgment of the treating physician and therapy team regarding the needs of the patient.

### Use of a Composition or Combination of the Present Invention Outside of Conventional Psychotherapy

In some embodiments, a composition or combination of the present invention is administered outside of conventional psychotherapy. This example method is a broader, more flexible approach to pharmacotherapy that is not centered on supervision by a therapist. These pharmacotherapy sessions can take place in many different quiet and safe settings, including the patient's home. The setting is typically chosen to offer a quiet setting, with minimal disruptions, where the patient feels psychologically safe and emotionally relaxed. The setting may be the patient's home but may alternatively be a clinic, retreat center, or hotel room.

In an alternative embodiment, the medicine is taken by the patient regularly to maintain therapeutic concentrations of the active compound in the blood. In another alternative embodiment, the medicine is taken, as needed, for defined psychotherapy sessions.

Optionally, a checklist may be followed to prepare the immediate environment to minimize distractions and maximize therapeutic or decision-making benefits. This checklist can include items such as silencing phones and other communications devices, cleaning and tidying the environment, preparing light refreshments, preparing playlists of appropriate music, and pre-arranging end-of-session transportation if the patient is not undergoing pharmacotherapy at home.

Before the pharmacotherapy session, there may be an initial determination of the therapeutic or other life-related goals (for example, decision-making, increasing creativity, or simply appreciation of life) that will be a focus of the session. These goals can optionally be determined in advance with support from a therapist.

Optionally, the therapist may help the patient select stimuli, such as photographs, videos, augmented or virtual reality scenes, or small objects such as personal possessions, that will help focus the patient's attention on the goals of the session or on the patient's broader life journey. As examples that are intended to be illustrative and not restrictive, these stimuli can include photographs of the patient from when they were young, which can increase self-compassion, or can include stimuli relating to traumatic events or phobias experienced by the patient, which can help the patient reevaluate and change their response to such stimuli. Optionally, the patient selects these stimuli without assistance (e.g., without the involvement of the therapist) or does not employ any stimuli. Optionally, stimuli are selected in real time by the therapist or an algorithm based on the events of the session with the goal of maximizing benefits to the patient.

If the patient is not experienced with the pharmacotherapy, a conversation occurs in which the therapist addresses the patient's questions and concerns about the medicine and familiarizes the patient with the logistics of a pharmacotherapy-assisted session. The therapist describes the kinds of experience that can be expected during the pharmacotherapy-assisted session. Optionally, parts of this conversation employ written, recorded, or interactive digital explanations, as might be used in the informed consent process in a clinical trial. The therapist may additionally make commitments to support the participant's healthcare and wellness process. In turn, the patient may be asked to make commitments of their own (such as not to hurt themselves or others and to abstain from contraindicated medicines or drugs for an adequate period before and after the pharmacotherapy).

Selected session goals and any commitments or other agreements regarding conduct between the patient and therapy team are reviewed immediately before administration of the medicine. Depending on the pharmaceutical preparation and route of administration, the therapeutic effects of the medicine usually begin within one hour. Typical therapeutic effects include decreased neuroticism and increased feelings of authenticity. Patients are often able to calmly contemplate experiences or possible experiences that would normally be upsetting or even overwhelming. This can facilitate decision making and creativity in addition to mental wellness.

Optionally, sleep shades and earphones with music or soothing noise may be used to reduce distractions from the environment. Optionally, a virtual reality or immersive reality system may be used to provide stimuli that support the therapeutic process. Optionally, these stimuli are preselected; optionally, they are selected in real time by a person or an algorithm based on events in the session with the goal of maximizing benefits to the patient. Optionally, a therapist or other person well-known to the patient is present or available nearby or via phone, video, or other communication method in case the patient wishes to talk, however the patient may optionally undergo a session without the assistance of a therapist. Optionally, the patient may write or create artwork relevant to the selected session goals. Optionally, the patient may practice stretches or other beneficial body movements, such as yoga (“movement activity”).

Optionally, in other embodiments the patient may practice movement activity that includes more vigorous body movements, such as dance or other aerobic activity. Movement activity also may make use of exercise equipment such as a treadmill or bicycle.

In some additional embodiments, the patient may be presented with music, video, auditory messages, or other perceptual stimuli. Optionally, these stimuli may be adjusted based on the movements or other measurable aspects of the patient. Such adjustment may be done by the therapist with or without the aid of a computer, or by a computer alone in response to said patient aspects, including by an algorithm or artificial intelligence, and “computer” broadly meaning any electronic tool suitable for such purposes, whether worn or attached to a patient (e.g., watches, fitness trackers, “wearables,” and other personal devices; biosensors or medical sensors; medical devices), whether directly coupled or wired to a patient or wirelessly connected (and including desktop, laptop, and notebook computers; tablets, smartphones, and other mobile devices; and the like), and whether within the therapy room or remote (e.g., cloud-based systems).

For example, measurable aspects of a patient (e.g., facial expression, eye movements, respiration rate, pulse rate, skin color change, patient voice quality or content, patient responses to questions) from these tools may be individually transformed into scores on standardized scales by subtracting a typical value and then multiplying by a constant and these scores may be further multiplied by constants and added together to create an overall score that can optionally be transformed by multiplication with a link function, such as the logit function, to create an overall score. This score may be used to select or adjust stimuli such as selecting music with higher or lower beats-per-minute or with faster or slower notes, selecting images, audio, or videos with different emotionality or autobiographical meaning, or selecting activities for the patient to engage in (such as specific movements, journaling prompts, or meditation mantras).

It should be readily appreciated that a patient can participate in numerous therapeutically beneficial activities, where such participation follows or is in conjunction with the administration of a compound or composition of the invention, including writing about a preselected topic, engaging in yoga or other movement activity, meditating, creating art, viewing of photographs or videos or emotionally evocative objects, using a virtual reality or augmented reality system, talking with a person, and thinking about a preselected problem or topic, and it should be understood that such participation can occur with or without the participation or guidance of a therapist.

Optionally, the prescribing physician may allow a second or even third administration of the medicine or another psychotherapeutic agent in order to extend the therapeutic effects. Optionally, a pharmaceutical preparation with modified release is employed to make this unnecessary.

The patient typically remains in the immediate environment until the acute therapeutic effects of the medicine are clinically minimal, usually within eight hours. After this point, the session is considered finished.

The treatment plan will often include a follow-up session with a therapist. This follow-up session occurs after the pharmacotherapy session has ended, often the next day but sometimes several days later. In this session, the patient discusses their experiences from the pharmacotherapy session with the therapist, who can aid them in recalling the therapeutic effects and help them to incorporate the experiences into their everyday lives.

Pharmacotherapy sessions may be repeated as needed, based on the judgment of the treating physician and therapy team regarding the needs of the patient.

### Pharmaceutical Compositions and Salts

Pharmaceutical compositions comprise a pharmaceutically acceptable carrier, diluent, or excipient, and at least one active ingredient, and in the case of this invention, typically two active agents. “Pharmaceutically acceptable” as used in connection with an excipient, carrier, or diluent means an excipient, carrier, or diluent that is useful in preparing a pharmaceutical composition that is generally safe, non-toxic, and neither biologically nor otherwise undesirable for veterinary use and/or human pharmaceutical use. Such compositions are prepared in a manner well known in the pharmaceutical art and comprise at least one active compound. (See, for example, Remington, 2005, Remington: The science and practice of pharmacy, 21st ed., Lippincott Williams & Wilkins.)

A “pharmaceutically acceptable composition” thus refers to at least one entactogen (which may be a mixture of enantiomers) and an SSRI or SNRI as described herein and a pharmaceutically acceptable vehicle, excipient, diluent, or other carrier, in an effective amount to treat a host, typically a human. The composition may be layered or otherwise engineered to accomplish the differential timing of release as discussed in detail herein.

In making the compositions employed in the present invention the active agents are usually mixed with an excipient, diluted by an excipient, or enclosed within such a carrier which can be in the form of a capsule, sachet, paper or other container. When the excipient serves as a diluent, it can be a solid, semi-solid, or liquid material, which acts as a vehicle, carrier, or medium for the active ingredient. Thus, the compositions can be in the form of tablets (including orally disintegrating, swallowable, sublingual, buccal, and chewable tablets), pills, powders, lozenges, troches, oral films, thin strips, sachets, cachets, soft and hard gelatin capsules containing for example about 10% by weight of the active agents. Compositions may be formulated as immediate release, controlled release, sustained (extended) release or modified release formulations to achieve the goals of the present invention.

In preparing a formulation, it may be necessary to mill the active compound to provide the appropriate particle size prior to combining with the other ingredients. If the active compound is substantially insoluble, it ordinarily is milled to a particle size of less than 200 mesh. If the active compound is substantially water soluble, the particle size is normally adjusted by milling to provide a substantially uniform distribution in the formulation, for example, about 40 mesh.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup, and methyl cellulose. The formulations can additionally include: lubricating agents such as talc, magnesium stearate, and mineral oil; wetting agents; emulsifying and suspending agents; preserving agents such as methyl- and propylhydroxybenzoates; sweetening agents; and flavoring agents. The compositions of the invention can be formulated to provide quick, sustained or delayed release of the active ingredient after administration to the patient by employing procedures known in the art.

It will be understood that the amount of the compounds actually administered will be determined by a physician, in light of the relevant circumstances, including the condition to be treated, the chosen route of administration, the actual compound or compounds administered, the age, weight, and response of the individual patient, and the severity of the patient's symptoms, and therefore the above dosage ranges are not intended to limit the scope of the invention in any way.

Generally, the pharmaceutical compositions of the invention may be administered and dosed in accordance with good medical practice, taking into account the method and scheduling of administration, prior and concomitant medications and medical supplements, the clinical condition of the individual patient and the severity of the underlying disease, the patient's age, sex, body weight, and other such factors relevant to medical practitioners, and knowledge of the particular compound(s) used. Starting and maintenance dosage levels thus may differ from patient to patient, for individual patients across time, and for different pharmaceutical compositions, but shall be able to be determined with ordinary skill.

The formulations of the present invention are typically oral dosage forms, for example oral solid dosage forms. Oral solid dosage forms may include but are not limited to, tablets, capsules, caplets, powders, pellets, multiparticulates, beads, spheres and/or any combinations thereof. The oral solid dosage forms may be formulated as immediate release, controlled release, sustained (extended) release, delayed release or modified release formulations.

The oral solid dosage forms of the present invention may also contain pharmaceutically acceptable excipients such as fillers, diluents, lubricants, surfactants, glidants, binders, dispersing agents, suspending agents, disintegrants, viscosity-increasing agents, film-forming agents, granulation aid, flavoring agents, sweetener, coating agents, solubilizing agents, and combinations thereof.

In some embodiments, the solid dosage forms of the present invention may be in the form of a tablet (including a suspension tablet, a fast-melt tablet, a bite-disintegration tablet, a rapid-disintegration tablet, an effervescent tablet, or a caplet), a pill, a powder (including a sterile packaged powder, a dispensable powder, or an effervescent powder), a capsule (including both soft or hard capsules, for example, capsules made from animal-derived gelatin or plant-derived HPMC, or “sprinkle capsules”), solid dispersion, solid solution, bioerodible dosage form, controlled release formulations, pulsatile release dosage forms, multiparticulate dosage forms, pellets, or granule. In other embodiments, the pharmaceutical formulation is in the form of a powder. In still other embodiments, the pharmaceutical formulation is in the form of a tablet, including a fast-melt tablet. Additionally, the pharmaceutical formulations of the present invention may be administered as a single capsule or in multiple capsule dosage form. In some embodiments, the pharmaceutical formulation is administered in two, or three, or four, capsules or tablets.

The pharmaceutical solid dosage forms described herein comprises an entactogen in combination with an SSRI or SNRI described herein and one or more pharmaceutically acceptable additives such as a compatible carrier, binder, complexing agent, ionic dispersion modulator, filling agent, suspending agent, flavoring agent, sweetening agent, disintegrating agent, dispersing agent, surfactant, lubricant, colorant, diluent, solubilizer, moistening agent, plasticizer, stabilizer, penetration enhancer, wetting agent, anti-foaming agent, antioxidant, preservative, or one or more combinations thereof.

In still other aspects, using standard coating procedures, such as those described in Remington's Pharmaceutical Sciences, 20th Edition (2000), a film coating is provided around the entactogen and SSRI or SNRI (active agents) of the present invention formulation. In one embodiment, some or all of the active agents of the present invention particles are coated. In another embodiment, some or all of the active agents of the present invention particles are microencapsulated or nanoencapsulated. In yet another embodiment, some or all of the active agents of the present invention are amorphous material coated and/or microencapsulated with inert excipients. In still another embodiment, the active agents of the present invention particles are not encapsulated and are uncoated.

Suitable carriers for use in the solid dosage forms described herein include acacia, gelatin, colloidal silicon dioxide, calcium glycerophosphate, calcium lactate, maltodextrin, glycerin, magnesium silicate, sodium caseinate, soy lecithin, sodium chloride, tricalcium phosphate, dipotassium phosphate, sodium stearoyl lactylate, carrageenan, monoglyceride, diglyceride, pregelatinized starch, hydroxypropylmethylcellulose, hydroxypropylmethylcellulose acetate stearate, sucrose, microcrystalline cellulose, lactose, mannitol and the like.

Suitable filling agents for use in the solid dosage forms described herein include lactose, calcium carbonate, calcium phosphate, dibasic calcium phosphate, calcium sulfate, microcrystalline cellulose (for example, Avicel®, Avicel® PH101, Avicel® PH102, Avicel® PH105, etc.), cellulose powder, dextrose, dextrates, dextrose, dextran, starches, pregelatinized starch, hydroxypropylmethylcellulose (HPMC), hydroxypropylmethylcellulose phthalate, hydroxypropylmethylcellulose acetate stearate (HPMCAS), sucrose, xylitol, lactitol, mannitol, sorbitol, sodium chloride, polyethylene glycol, and the like.

If needed, suitable disintegrants for use in the solid dosage forms described herein include natural starch such as corn starch or potato starch, a pregelatinized starch such as National 1551 or Amijel®, or a sodium starch glycolate such as Promogel® or Explotab®, a cellulose such as a wood product, microcrystalline cellulose, for example, Avicel®, Avicel® PH101, Avicel® PH102, Avicel® PH105, Elcema® P100, Emcocel®, Vivacel®, Ming Tia®, and Solka-Floc®, Ac-Di-Sol, methylcellulose, croscarmellose, or a cross-linked cellulose, such as cross-linked sodium carboxymethylcellulose (Ac-Di-Sol®), cross-linked carboxymethylcellulose, or cross-linked croscarmellose, a cross-linked starch such as sodium starch glycolate, a cross-linked polymer such as crosspovidone, a cross-linked polyvinylpyrrolidone, alginate such as alginic acid or a salt of alginic acid such as sodium alginate, a clay such as Veegum® HV (magnesium aluminum silicate), a gum such as agar, guar, locust bean, Karaya, pectin, or tragacanth, sodium starch glycolate, bentonite, a natural sponge, a surfactant, a resin such as a cation-exchange resin, citrus pulp, sodium lauryl sulfate, sodium lauryl sulfate in combination starch, and the like.

Binders impart cohesiveness to solid oral dosage form formulations: for powder-filled capsule formulation, they aid in plug formation that can be filled into soft- or hard-shell capsules and in tablet formulation, binders ensure that the tablet remains intact after compression and help assure blend uniformity prior to a compression or fill step. Materials suitable for use as binders in the solid dosage forms described herein include carboxymethylcellulose, methylcellulose (for example, Methocel®), hydroxypropylmethylcellulose (for example, Hypromellose USP Pharmacoat-603, hydroxypropylmethylcellulose acetate stearate (Agoate HS-LF and HS), hydroxyethylcellulose, hydroxypropylcellulose (for example, Klucel®), ethylcellulose (for example, Ethocel®), and microcrystalline cellulose (for example, Avicel®), microcrystalline dextrose, amylose, magnesium aluminum silicate, polysaccharide acids, bentonites, gelatin, polyvinylpyrrolidone/vinyl acetate copolymer, crosspovidone, povidone, starch, pregelatinized starch, tragacanth, dextrin, a sugar, such as sucrose (for example, Dipac®), glucose, dextrose, molasses, mannitol, sorbitol, xylitol (for example, Xylitab®), lactose, a natural or synthetic gum such as acacia, tragacanth, ghatti gum, mucilage of isapol husks, starch, polyvinylpyrrolidone (for example, Povidone® CL, Kollidon® CL, Polyplasdone® XL-10, and Povidone® K-12), larch arabogalactan, Veegum®, polyethylene glycol, waxes, sodium alginate, and the like. In general, binder levels of 20-70% are typically used in powder-filled gelatin capsule formulations. Binder usage level in tablet formulations is a function of whether direct compression, wet granulation, roller compaction, or usage of other excipients such as fillers which itself can act as moderate binders are used. Formulators skilled in the art can determine the binder level for the formulations, but binder usage level of up to 70% in tablet formulations is common.

Suitable lubricants or glidants for use in the solid dosage forms described herein include stearic acid, calcium hydroxide, talc, corn starch, sodium stearyl fumarate, alkali-metal and alkaline earth metal salts, such as aluminum, calcium, magnesium, zinc, stearic acid, sodium stearates, magnesium stearate, zinc stearate, waxes, Stearowet®, boric acid, sodium benzoate, sodium acetate, sodium chloride, leucine, a polyethylene glycol or a methoxypolyethylene glycol such as Carbowax™, PEG 4000, PEG 5000, PEG 6000, propylene glycol, sodium oleate, glyceryl behenate, glyceryl palmitostearate, glyceryl benzoate, magnesium or sodium lauryl sulfate, and the like.

Suitable diluents for use in the solid dosage forms described herein include sugars (including lactose, sucrose, and dextrose), polysaccharides (including dextrates and maltodextrin), polyols (including mannitol, xylitol, and sorbitol), cyclodextrins and the like.

Non-water-soluble diluents are compounds typically used in the formulation of pharmaceuticals, such as calcium phosphate, calcium sulfate, starches, modified starches and microcrystalline cellulose, and micro cellulose (for example, having a density of about 0.45 g/cm³, for example Avicel®, powdered cellulose), and talc.

Suitable wetting agents for use in the solid dosage forms described herein include oleic acid, glyceryl monostearate, sorbitan monooleate, sorbitan monolaurate, triethanolamine oleate, polyoxyethylene sorbitan monooleate, polyoxyethylene sorbitan monolaurate, quaternary ammonium compounds (for example, Polyquat 10®), sodium oleate, sodium lauryl sulfate, magnesium stearate, sodium docusate, triacetin, vitamin E TPGS and the like. Wetting agents include surfactants.

Suitable surfactants for use in the solid dosage forms described herein include docusate and its pharmaceutically acceptable salts, sodium lauryl sulfate, sorbitan monooleate, polyoxyethylene sorbitan monooleate, polysorbates, poloxamers, bile salts, glyceryl monostearate, copolymers of ethylene oxide and propylene oxide, for example, Pluronic® (BASF), and the like.

Suitable suspending agents for use in the solid dosage forms described here include polyvinylpyrrolidone, for example, polyvinylpyrrolidone K12, polyvinylpyrrolidone K17, polyvinylpyrrolidone K25, or polyvinylpyrrolidone K30, polyethylene glycol, for example, the polyethylene glycol can have a molecular weight of about 300 to about 6000, or about 3350 to about 4000, or about 7000 to about 18000, vinylpyrrolidone/vinyl acetate copolymer (S630), sodium alginate, gums, such as, for example, gum tragacanth and gum acacia, guar gum, xanthans, including xanthan gum, sugars, cellulosic, such as, for example, sodium carboxymethylcellulose, methylcellulose, sodium carboxymethylcellulose, hydroxypropylmethylcellulose, hydroxyethylcellulose, polysorbate-80, polyethoxylated sorbitan monolaurate, polyethoxylated sorbitan monolaurate, povidone and the like.

Suitable antioxidants for use in the solid dosage forms described herein include, for example, butylated hydroxytoluene (BHT), butyl hydroxyanisole (BHA), sodium ascorbate, Vitamin E TPGS, ascorbic acid, sorbic acid and tocopherol.

Immediate-release components of the formulation may be prepared by combining superdisintegrants such as Croscarmellose sodium and different grades of microcrystalline cellulose in different ratios. To aid disintegration, sodium starch glycolate will be added.

The above-listed additives should be taken as merely examples and not limiting, of the types of additives that can be included in solid dosage forms of the present invention. The amounts of such additives can be readily determined by one skilled in the art, according to the particular properties desired.

The co-formulated tablets of the invention described here can be prepared by methods known in the art. Various methods for the preparation of the immediate release, modified release, controlled release, and extended-release dosage forms (for example, as matrix tablets, tablets having one or more modified, controlled, or extended-release layers, etc.) and the vehicles therein are well known in the art. Generally recognized compendia of methods include: Remington: The Science and Practice of Pharmacy, Alfonso R. Gennaro, Editor, 20th Edition, Lippincott Williams & Wilkins, Philadelphia, PA; and Sheth et al. (1980), Compressed tablets, in Pharmaceutical dosage forms, Vol. 1, edited by Lieberman and Lachtman, Dekker, NY.

In certain embodiments, solid dosage forms, for example tablets, effervescent tablets, and capsules, are prepared by mixing the active agents of the present invention particles with one or more pharmaceutical excipients to form a bulk blend composition. When referring to these bulk blend compositions as homogeneous, it is meant that the active agents of the present invention particles are dispersed evenly throughout the composition so that the composition may be readily subdivided into equally effective unit dosage forms, such as tablets, pills, and capsules. The individual unit dosages may also comprise film coatings, which disintegrate upon oral ingestion or upon contact with diluents. These the active agents of the present invention formulations can be manufactured by conventional pharmaceutical techniques.

Conventional pharmaceutical techniques for preparation of solid dosage forms include, for example, one or a combination of methods: (1) dry mixing, (2) direct compression, (3) milling, (4) dry or non-aqueous granulation, (5) wet granulation, or (6) fusion. See, for example, Lachman et al., Theory and Practice of Industrial Pharmacy (1986). Other methods include, for example, spray drying, pan coating, melt granulation, granulation, fluidized bed spray drying or coating (for example, Wurster coating), tangential coating, top spraying, tableting, extruding and the like.

Compressed tablets are solid dosage forms prepared by compacting the bulk blend of the active agents of the present invention formulations described above. In various embodiments, compressed tablets which are designed to dissolve in the mouth may comprise one or more flavoring agents. In other embodiments, the compressed tablets will comprise a film surrounding a final compressed tablet. In some embodiments, the film coating can provide a delayed release of the active agents of the present invention formulation. In other embodiments, the film coating aids in patient compliance (for example, Opadry® coatings or sugar coating). Film coatings comprising Opadry® typically range from about 1% to about 3% of the tablet weight. Film coatings for delayed-release usually comprise 2-6% of a tablet weight or 7-15% of a spray-layered bead weight. In other embodiments, the compressed tablets comprise one or more excipients.

A capsule may be prepared, for example, by placing the bulk blend of the active agents of the present invention formulation, described above, inside of a capsule. In some embodiments, the formulations of the present invention (non-aqueous suspensions and solutions) are placed in a soft gelatin capsule. In other embodiments, the formulations of the present invention are placed in standard gelatin capsules or non-gelatin capsules such as capsules comprising HPMC. In other embodiments, the formulations of the present invention are placed in a sprinkle capsule, wherein the capsule may be swallowed whole, or the capsule may be opened, and the contents sprinkled on food prior to eating. In some embodiments of the present invention, the therapeutic dose is split into multiple (for example, two, three, or four) capsules. In some embodiments, the entire dose of the active agents of the present invention is delivered in a capsule form.

In certain embodiments, ingredients (including or not including the active agents) of the invention are wet granulated. The individual steps in the wet granulation process of tablet preparation include milling and sieving of the ingredients, dry powder mixing, wet massing, granulation, drying, and final grinding. In various embodiments, the active agents of the present invention composition are added to the other excipients of the pharmaceutical formulation after they have been wet granulated. Alternatively, the ingredients may be subjected to dry granulation, for example, via compressing a powder mixture into a rough tablet or “slug” on a heavy-duty rotary tablet press. The slugs are then broken up into granular particles by a grinding operation, usually by passage through an oscillation granulator. The individual steps include mixing of the powders, compressing (slugging) and grinding (slug reduction or granulation). No wet binder or moisture is involved in any of the steps.

In some embodiments, the active agents of the present invention formulation are dry granulated with other excipients in the pharmaceutical formulation. In other embodiments, the active agents of the present invention formulation are added to other excipients of the pharmaceutical formulation after they have been dry granulated.

In other embodiments, the formulation of the present invention formulations described herein is a solid dispersion. Methods of producing such solid dispersions are known in the art and include U.S. Pat. Nos. 4,343,789; 5,340,591; 5,456,923; 5,700,485; 5,723,269; and U.S. Pub. No. 2004/0013734. In some embodiments, the solid dispersions of the invention comprise both amorphous and non-amorphous active agents of the present invention and can have enhanced bioavailability as compared to conventional active agents of the present invention formulations. In still other embodiments, the active agents of the present invention formulations described herein are solid solutions. Solid solutions incorporate a substance together with the active agent and other excipients such that heating the mixture results in the dissolution of the drug and the resulting composition is then cooled to provide a solid blend that can be further formulated or directly added to a capsule or compressed into a tablet. In certain embodiments, a fixed dose formulation is provided that uses a lower dose of active agents than a formulation provided herein. For example, in certain embodiments a half dose formulation is provided, for example, the fixed dose combination described in Example 2, wherein the entactogen granules are formulated with about 15 mg of entactogen, and SSRI or SNRI enteric beads are formulation with about 12 mg of SSRI or SNRI.

### Modified-Release Formulations

The pharmaceutical coformulation of the present invention, for example, an oral solid dosage form, may contain a suitable amount of modified-release agents (for example, delayed-release agents) to insure differential and delayed release of the SSRI or SNRI. The pharmaceutical solid oral dosage forms comprising the active agents of the present invention described herein can be alternatively formulated to provide a modified or controlled release of both of the active agents of the present invention, as long as there is a kinetic lag between the release of the entactogen and the release of the SSRI or SNRI. In some embodiments, the solid dosage forms described herein can be co-formulated differential delayed release dosage forms in an enteric-coating, i.e., as an oral dosage form of a pharmaceutical composition as described herein which uses an enteric coating to affect release of one or both of the components in the small intestine of the gastrointestinal tract. The enteric-coated dosage form may be a compressed or molded or extruded tablet/mold (coated or uncoated) containing granules, powder, pellets, beads or particles of the active ingredient and/or other composition components, which are themselves coated or uncoated. The enteric coated oral dosage form may also be a capsule (coated or uncoated) containing pellets, beads or granules of the solid carrier or the composition, which are themselves coated or uncoated.

In other embodiments, an entactogen and an SSRI or SNRI in the formulations described herein can be delivered using a pulsatile dosage form. Pulsatile dosage forms comprising the active agents of the present invention described herein may be administered using a variety of formulations known in the art. For example, such formulations include those described in U.S. Pat. Nos. 5,011,692; 5,017,381; 5,229,135; and 5,840,329. Other dosage forms suitable for use with the active agents of the present invention are described in, for example, U.S. Pat. Nos. 4,871,549; 5,260,068; 5,260,069; 5,508,040; 5,567,441; and 5,837,284.

In certain embodiments, the controlled release dosage form is pulsatile release solid oral dosage form comprising at least two groups of particles, each containing active agents of the present invention as described herein. The first group of particles provides a substantially immediate dose of the active agents of the present invention upon ingestion by a subject. The first group of particles can be either uncoated or comprise a coating and/or sealant. The second group of particles comprises coated particles, which may comprise from at least about 2% to about 75% or less, typically from at least about 2.5% to about 70% or less, or from at least about 40% to about 70% or less, by weight of the total dose of the active agents of the present invention in the formulation, in admixture with one or more binders.

In one embodiment, a coating for providing a delayed release is applied to a compound described herein or to a core containing a compound described herein.

The coating of one or both of the components, or the entire dosage form, may comprise a pharmaceutically acceptable ingredient in an amount sufficient, for example, to provide an extended release following ingestion before release of the active agent. Suitable coatings include one or more differentially degradable coatings such as, by way of example only, pH-sensitive coatings (enteric coatings) such as acrylic resins (for example, Eudragit® EPO, Eudragit® L30D-55, Eudragit® FS 30D Eudragit® L100-55, Eudragit® L100, Eudragit® S100, Eudragit® RD100, Eudragit® E100, Eudragit® L12.5, Eudragit® S12.5, and Eudragit® NE30D, Eudragit® NE 40D®) either alone or blended with cellulose derivatives, for example, ethylcellulose, or non-enteric coatings having variable thickness to provide differential release of the active agents of the present invention formulation.

Many other types of modified (e.g. delayed) release systems known to those of ordinary skill in the art and are suitable for use with the active agents of the present invention formulations described herein. Examples of such delivery systems include polymer-based systems, such as polylactic and polyglycolic acid, polyanhydrides and polycaprolactone, cellulose derivatives (for example, ethylcellulose), porous matrices, nonpolymer-based systems that are lipids, including sterols, such as cholesterol, cholesterol esters and fatty acids, or neutral fats, such as mono-, di- and triglycerides; hydrogel release systems; silastic systems; peptide-based systems; wax coatings, bioerodible dosage forms, compressed tablets using conventional binders and the like. See, for example, Liberman et al., Pharmaceutical Dosage Forms, 2 Ed., Vol. 1, pp. 209-214 (1990); Singh et al., Encyclopedia of Pharmaceutical Technology, 2nd Ed., pp. 751-753 (2002); U.S. Pat. Nos. 4,327,725; 4,624,848; 4,968,509; 5,461,140; 5,456,923, 5,516,527; 5,622,721, 5,686,105; 5,700,410; 5,977,175; 6,465,014 and 6,932,983.

In certain embodiments, the controlled release systems may comprise the modified (e.g. delayed) release material incorporated with the drug(s) into a matrix, whereas in other formulations, the controlled release material may be applied to a core containing the drug(s). In certain embodiments, one drug may be incorporated into the core while the other drug is incorporated into the coating. In some embodiments, materials include shellac, acrylic polymers, cellulosic derivatives, polyvinyl acetate phthalate, and mixtures thereof. In other embodiments, materials include Eudragit® series E, L, RL, RS, NE, L, L300, S, 100-55, cellulose acetate phthalate, Aquateric, cellulose acetate trimellitate, ethyl cellulose, hydroxypropylmethylcellulose phthalate, hydroxypropylmethylcellulose acetate succinate, polyvinyl acetate phthalate, and Cotteric.

The modified (e.g. delayed) release systems may use a hydrophilic polymer, including a water-swellable polymer (for example, a natural or synthetic gum). The hydrophilic polymer may be any pharmaceutically acceptable polymer which swells and expands in the presence of water to slowly release the active agents of the present invention. These polymers include polyethylene oxide, methylcellulose, hydroxypropyl cellulose, hydroxypropylmethylcellulose, and the like.

The performance of acrylic polymers (primarily their solubility in biological fluids) can vary based on the degree and type of substitution. Examples of suitable acrylic polymers which may be used in matrix formulations or coatings include methacrylic acid copolymers and ammonia methacrylate copolymers. The Eudragit series E, L, S, RL, RS and NE (Rohm Pharma) are available as solubilized in an organic solvent, aqueous dispersion, or dry powders. The Eudragit series RL, NE, and RS are insoluble in the gastrointestinal tract but are permeable and are used primarily for colonic targeting. The Eudragit series E dissolve in the stomach. The Eudragit series L, L-30D and S are insoluble in the stomach and dissolve in the intestine; Opadry Enteric is also insoluble in the stomach and dissolves in the intestine.

Examples of suitable cellulose derivatives for use in matrix formulations or coatings include ethyl cellulose; reaction mixtures of partial acetate esters of cellulose with phthalic anhydride. The performance can vary based on the degree and type of substitution. Cellulose acetate phthalate (CAP) dissolves in pH>6. Aquateric (FMC) is an aqueous-based system and is a spray-dried CAP psuedolatex with particles<1 μm. Other components in Aquateric can include pluronic, Tweens, and acetylated monoglycerides. Other suitable cellulose derivatives include cellulose acetate trimellitate (Eastman); methylcellulose (Pharmacoat, Methocel); hydroxypropylmethylcellulose phthalate (HPMCP); hydroxypropylmethylcellulose succinate (HPMCS); and hydroxypropylmethylcellulose acetate succinate (for example, AQOAT (Shin Etsu)). The performance can vary based on the degree and type of substitution. For example, HPMCP such as, HP-50, HP-55, HP-555, HP-55F grades are suitable. The performance can vary based on the degree and type of substitution. For example, suitable grades of hydroxypropylmethylcellulose acetate succinate include AS-LG (LF), which dissolves at pH 5, AS-MG (MF), which dissolves at pH 5.5, and AS-HG (1F), which dissolves at higher pH. These polymers are offered as granules or as fine powders for aqueous dispersions. Other suitable cellulose derivatives include hydroxypropylmethylcellulose.

In some embodiments, the coating may contain a plasticizer and possibly other coating excipients such as colorants, talc, and/or magnesium stearate, which are well known in the art. Suitable plasticizers include triethyl citrate (Citroflex 2), triacetin (glyceryl triacetate), acetyl triethyl citrate (Citroflec A2), Carbowax 400 (polyethylene glycol 400), diethyl phthalate, tributyl citrate, acetylated monoglycerides, glycerol, fatty acid esters, propylene glycol, and dibutyl phthalate. In particular, anionic carboxylic acrylic polymers usually will contain 10-25% by weight of a plasticizer, especially dibutyl phthalate, polyethylene glycol, triethyl citrate, and triacetin. Conventional coating techniques such as spray or pan coating are employed to apply coatings. The coating thickness must be sufficient to ensure that the oral dosage form remains intact until the desired site of topical delivery in the intestinal tract is reached.

Multilayer tablet delivery (for example, such as that used in the GeoMatrix™ technology) comprises a hydrophilic matrix core containing the active ingredient and one or two impermeable or semi-permeable polymeric coatings. This technology uses films or compressed polymeric barrier coatings on one or both sides of the core. The presence of polymeric coatings (for example, such as that used in the GeoMatrix™ technology) modifies the hydration/swelling rates of the core and reduces the surface area available for drug release. These partial coatings provide modulation of the drug dissolution profile: they reduce the release rate from the device and shift the typical time-dependent release rate toward constant release. This technology enables customized levels of controlled release of specific active agents and/or simultaneous release of two different active agents at different rates that can be achieved from a single tablet. The combination of layers, each with different rates of swelling, gelling and erosion, is used for the rate of drug release in the body. Exposure of the multilayer tablet as a result of the partial coating may affect the release and erosion rates, therefore, transformation of a multilayered tablet with exposure on all sides to the gastrointestinal fluids upon detachment of the barrier layer will be considered.

Multi-layered tablets containing combinations of immediate release and modified/extended release of two different active agents or dual release rate of the same drug in a single dosage form may be prepared by using hydrophilic and hydrophobic polymer matrices. Dual release repeat action multi-layered tablets may be prepared with an outer compression layer with an initial dose of rapidly disintegrating matrix in the stomach and a core inner layer tablet formulated with components that are insoluble in the gastric media but release efficiently in the intestinal environment.

In certain embodiments, the dosage form is a solid oral dosage form which is an immediate release dosage form whereby >80% of the active agents of the present invention are released within 1 or 2 hours after administration. In other embodiments, the invention provides an (for example, solid oral) dosage form that is a controlled release or pulsatile release dosage form. In such instances, the release may be, for example, 30 to 60% of the active agents of the present invention particles by weight are released from the dosage form within about 2 hours after administration and about 90% by weight of the active agents of the present invention released from the dosage form, for example, within about 4 hours after administration. In yet other embodiments, the dosage form includes at least one active agent in an immediate-release form and at least one active agent in the delayed-release form or sustained-release form. In yet other embodiments, the dosage form includes at least two active agents that are released at different rates as determined by in-vitro dissolution testing or via oral administration.

The various release dosage formulations discussed above, and others known to those skilled in the art can be characterized by their disintegration profile. A profile is characterized by the test conditions selected. Thus, the disintegration profile can be generated at a pre-selected apparatus type, shaft speed, temperature, volume, and pH of the dispersion media. Several disintegration profiles can be obtained. For example, a first disintegration profile can be measured at a pH level approximating that of the stomach (about pH 1.2); a second disintegration profile can be measured at a pH level approximating that of one point in the intestine or several pH levels approximating multiple points in the intestine (about 6.0 to about 7.5, more specifically, about 6.5 to 7.0). Another disintegration profile can be measured using distilled water. The release of formulations may also be characterized by their pharmacokinetic parameters, for example, Cmax, Tₘₐₓ, and AUC (0-τ).

In certain embodiments, the controlled, delayed or extended-release of one or more of the active agents of the fixed-dose combinations of the invention may be in the form of a capsule having a shell comprising the material of the rate-limiting membrane, including any of the coating materials previously discussed, and filled with the active agents of the present invention particles. A particular advantage of this configuration is that the capsule may be prepared independently of the active agent of the present invention particles; thus, process conditions that would adversely affect the drug can be used to prepare the capsule.

Alternatively, the formulation may comprise a capsule having a shell made of a porous or a pH-sensitive polymer made by a thermal forming process. Another alternative is a capsule shell in the form of an asymmetric membrane, i.e., a membrane that has a thin skin on one surface and most of whose thickness is constituted of a highly permeable porous material. The asymmetric membrane capsules may be prepared by a solvent exchange phase inversion, wherein a solution of polymer, coated on a capsule-shaped mold, is induced to phase separate by exchanging the solvent with a miscible non-solvent. In another embodiment, spray layered active agents of the present invention particles are filled in a capsule.

An exemplary process for manufacturing the spray layered active agents of the present invention is the fluidized bed spraying process. The active agents of the present invention suspensions or the active agents of the present invention complex suspensions described above may be sprayed onto sugar or microcrystalline cellulose (MCC) beads (20-35 mesh) with Wurster column insert at an inlet temperature of 50° C. to 60° C. and air temp of 30° C. to 50° C. A 15 to 20 wt % total solids content suspension containing 45 to 80 wt % the active agents of the present invention, 10 to 25 wt % hydroxymethylpropylcellulose, 0.25 to 2 wt % of SLS, 10 to 18 wt % of sucrose, 0.01 to 0.3 wt % simethicone emulsion (30% emulsion) and 0.3 to 10% NaCl, based on the total weight of the solid content of the suspension, are sprayed (bottom spray) onto the beads through 1.2 mm nozzles at 10 mL/min and 1.5 bar of pressure until a layering of 400 to 700% wt % is achieved as compared to initial beads weight. The resulting spray layered the active agents of the present invention particles, or the active agents of the present invention complex particles comprise about 30 to 70 wt % of the active agents of the present invention based on the total weight of the particles.

In one embodiment the capsule is a size 0 soft gelatin capsule. In one embodiment, the capsule is a swelling plug device. In another embodiment, the swelling plug device is further coated with cellulose acetate phthalate or copolymers of methacrylic acid and methylmethacrylate. In some embodiments, the capsule includes an effective amount of each of the active agents and and has a total weight of less than 800 mg (or less than 750 or 700 mg). The capsule may contain a plurality of the active agents of the present invention-containing beads, for example, spray layered beads. In some embodiments, the beads are 12-25% the active agents of the present invention by weight. In some embodiments, some or all of the active agents of the present invention containing beads are coated with a coating comprising 6 to 15% (or 8 to 12%) of the total bead weight. Optimization work typically involves lower loading levels, and the beads constitute 30 to 60% of the finished bead weight. The capsule may contain a granulated composition, wherein the granulated composition comprises the active agents of the present invention.

The capsule may provide pulsatile release of the active agents of the present invention oral dosage form. In one embodiment, the formulations comprise: (a) a first dosage unit comprising an entactogen described herein that is released substantially immediately following oral administration of the dosage form to a patient; (b) a second dosage unit comprising an SSRI or SNRI described herein that is released approximately 1 to 8 hours following administration of the dosage form to a patient, and more typically 3 to 7 or 8, or 4 to 7 or 8 hours after release of the entactogen.

For pulsatile release capsules containing beads, the beads can be coated with a coating comprising 6 to 15% (or 8 to 12%) of the total bead weight. In some embodiments, the coating is a coating that is insoluble at pH 1 to 2 and soluble at pH greater than 5.5. In other embodiments, the pulsatile release capsule contains a plurality of beads formulated for modified release and the at least one agent of the present invention is, for example, spray granulated for immediate release.

In some embodiments, the release of the active agents of the present invention particles can be modified with a modified release coating, such as an enteric coating using cellulose acetate phthalate or a sustained release coating comprising copolymers of methacrylic acid and methylmethacrylate. In some embodiments, the spray layered particles coated with the delayed and/or sustained release coatings can be filled in a modified release capsule in which both enteric-coated particles and immediate release particles of the present invention beads are filled into a soft gelatin capsule. Additional suitable excipients may also be filled with the coated particles in the capsule. The uncoated particles release the active agent of the present invention immediately upon administration while the coated particles do not release the active agent of the present invention until these particles reach the intestine. By controlling the ratios of the coated and uncoated particles, desirable pulsatile release profiles also may be obtained.

In certain embodiments, spray layered active agents of the present invention can be compressed into tablets with commonly used pharmaceutical excipients. Any appropriate apparatus for forming the coating can be used to make the enteric coated tablets, for example, fluidized bed coating using a Wurster column, powder layering in coating pans or rotary coaters; dry coating by double compression technique; tablet coating by film coating technique, and the like. See, for example, U.S. Pat. No. 5,322,655; Remington's Pharmaceutical Sciences Handbook: Chapter 90 “Coating of Pharmaceutical Dosage Forms,” 1990.

In other embodiments, the spray layered active agents of the present invention particles or spray layered active agents complex particles with enteric coatings described above and one or more excipients are dry blended and compressed into a mass, such as a tablet.

In certain embodiments, a pulsatile release of the active agent of the present invention formulation comprises a first dosage unit comprising a formulation made from the active agent of the present invention containing granules made from a spray drying or spray granulated procedure or a formulation made from the active agent of the present invention complex containing granules made from a spray drying or spray granulated procedure without enteric or sustained-release coatings and a second dosage unit comprising spray layered the active agent of the present invention particles or spray layered the active agent of the present invention complex particles with enteric or sustained-release coatings. In one embodiment, the active agent is wet or dry blended and compressed into a mass to make a pulsatile release tablet.

In certain embodiments, binding, lubricating and disintegrating agents are blended (wet or dry) to the spray layered active agent of the present invention to make a compressible blend. In one embodiment, the dosage unit containing a compound described herein and the dosage unit containing the other pharmacological agent are compressed separately and then compressed together to form a bilayer tablet. In yet another embodiment, the dosage unit containing the other pharmacological agent is in the form of an overcoat and completely covers the second dosage unit containing a compound described herein. In yet another embodiment, the dosage unit containing a compound described herein is in the form of an overcoat and completely covers the second dosage unit containing the other pharmacological agent.

In one embodiment, the dosage unit containing a compound of any of a compound described herein and the dosage unit containing the other pharmacological agent are compressed separately and then compressed together to form a bilayer tablet. In yet another embodiment, the dosage unit containing the other pharmacological agent is in the form of an overcoat and completely covers the second dosage unit containing a compound described herein. In yet another embodiment, the dosage unit containing a compound described herein is in the form of an overcoat and completely covers the second dosage unit containing the other pharmacological agent.

### Bilayer Tablets

In certain aspects the engineered formulation of the present invention is a bilayer tablet comprising the entactogen in the outer layer and the SSRI or SNRI in the inner layer.

In certain embodiments the outer layer comprises the entactogen and a water-soluble diluent. Non-limiting examples of water-soluble diluents include monosaccharides, for example glucose, oligosaccharides, for example sucrose or lactose, and sugar alcohols, for example sorbitol, mannitol, dulcitol, ribitol, or xylitol. In certain embodiments the outer layer comprises one or more disintegrants. Non-limiting examples of disintegrants include crospovidone, starch, maize starch, pregelatinized starch, hydroxypropyl starch sodium and/or calcium salts of carboxymethyl cellulose, cross-linked carboxymethylcellulose, polacrilin potassium, low substituted hydroxypropylcellulose, sodium and/or calcium alginate, docusate sodium, methylcellulose, agar, guar gum, chitosan, alginic acid and mixtures thereof, preferably from crospovidone, carboxymethylcellulose sodium, and croscarmellose sodium.

In certain embodiments the concentration of croscarmellose sodium or crospovidone in the outer layer is between about 1 to about 10, 15 or 20% by weight.

In certain embodiments the inner layer comprises an SSRI or SNRI and a gel forming polymer. In certain embodiments the gel forming polymer is selected from sodium carboxymethylcellulose anionic polysaccharides, anionic gums, polymers of acrylic acid, carbomers, crosslinked copolymers of acrylic acid and a hydrophobic C₀-C₃₀ alkyl acrylate co-monomers, and polycarbophils. In certain embodiments the gel forming polymer is selected from HPMC K100 LV, HPMC K100M and HPMC K4M.

In certain aspects the outer layer further comprises an extragranular and intragranular layer. In certain aspects the inner layer further comprises an extragranular and intragranular layer.

Non-limiting examples of procedures for preparing bilayer tablets for delayed release include those described in WO2023/001880, WO2019/212426, and Kopeček J. Adv Drug Deliv Rev. 2013 January; 65(1):49-59.

### Controlled Release Tri-Layer Tablet

In certain aspects the engineered formulation of the present invention is a trilayer tablet. In certain embodiments a tri-layer tablet is used for controlled release of a SSRI or SNRI following the release of an entactogen. The outer layer comprises an effective amount of entactogen or a pharmaceutically acceptable salt thereof, and additional pharmaceutically acceptable excipients, which is immediately released in gastric environment. The second layer comprises a protective coating which protects the inner most layer during upper gastric transit and starts to dissolve at the pH of the upper small intestine (approx. pH 5.5). In certain embodiments, the second layer is composed of Eudragit L30 D-55, Eudragit L-100, Eudragit S-100 and/or HPMC E-50LV. The inner layer comprises an effective amount of a SSRI or SNRI. Following the immediate release of the entactogen, the coating starts to swell and dissolve in duodenum releasing the SSRI or SNRI.

In certain embodiments, the SSRI or SNRI is formulated with delayed release excipients, for example, cellulose acetate, phthalate, hydroxylpropylmethylcellulose, hydroxypropylcellulose, methacrylic acid-ethyl acrylate, or other common methacrylate polymers to further delay the release.

### Controlled Release Dual Coat Tablet

In certain embodiments, a dual coat tablet is used for controlled release of an SSRI or SNRI following the release of an entactogen. The outer layer comprises an effective amount of entactogen or a pharmaceutically acceptable salt thereof, and additional pharmaceutically acceptable excipients, which is immediately released in gastric environment. The second layer comprises a protective coating which protects the inner most layer during upper gastric transit and starts to dissolve at the pH of the upper small intestine (approx. pH 5.5). In certain embodiments, the second layer is composed of Eudragit L30 D-55, Eudragit L-100, Eudragit S-100 and/or HPMC E-50LV. The third layer is composed of modified coating of the second layer, which is neutralized by addition of sodium hydroxide. The inner layer comprises an effective amount of a SSRI or SNRI. Following the immediate release of the entactogen, at pH 5.5, the second layer (outer coating) starts to swell and dissolve in duodenum. Once the outer layer is dissolved, intestinal fluid penetrates into the system and reaches the neutralized inner coating, which rapidly dissolves releasing the drug.

In certain embodiments, an SSRI or SNRI is formulated with delayed release excipients, for example, cellulose acetate, phthalate, hydroxypropylcellulose, methacrylic acid-ethyl acrylate, or other common methacrylate polymers to further delay the release.

Non-limiting examples of methods to prepare dual coat tablets include those described in Liu F et al. J Control Release. 2010 Oct. 15; 147(2):242-5 which exemplifies the use of DuoCoat technology.

### Chemically Controlled Delivery of SSRI or SNRI

In certain embodiments, the delayed release of the SSRI or SNRI is achieved by using a chemically controlled delivery technology. For example, in certain embodiments a bilayer tablet is formulated, wherein the outer layer comprises an effective amount of entactogen or a pharmaceutically acceptable salt thereof, and additional pharmaceutically acceptable excipients, and the inner layer comprises a polymer-SSRI/SNRI drug dispersion system or polymer-SSRI/SNRI drug conjugate system.

In certain embodiments, an SSRI or SNRI is uniformly dispersed/dissolved in a biodegradable polymer, wherein following the release of the entactogen from the outer layer, the inner layer biodegrades via bulk erosion or surface erosion, releasing the SSRI or SNRI.

In certain other embodiments, an SSRI or SNRI is chemically conjugated with polymer by covalent bonding or grafting to the polymer backbone, wherein following the release of the entactogen from the outer layer, the SSRI or SNRI releases through cleavage of polymer-SSRI/SNRI bonds under physiological pH.

In certain embodiments, a hydrogel delivery system is used, wherein an entactogen is formulated in a highly porous hydrogel, which allows free diffusion of the entactogen, and an SSRI/SNRI is formulated in a low porosity hydrogel, which requires network erosion for release.

In certain embodiments, an SSRI or SNRI is encapsulated in nanospheres, or nanocapsules (5 nm-1000 nm), and formulated with granules of entactogen to allow kinetic lag.

Non-limiting examples of methods to prepare formulations to chemically control the release of the SSRI or SNRI include those described in Kopeček J. Adv Drug Deliv Rev. 2013 January; 65(1):49-59.

### Nanocarriers and Hydrogels

In certain embodiments, a hydrogel delivery system is used, wherein an entactogen is formulated in highly porous hydrogel, which allows free diffusion of the entactogen, and an SSRI/SNRI is formulated in a low porosity hydrogel, which require network erosion for release.

In certain embodiments, an SSRI or SNRI is encapsulated in nanospheres, or nanocapsules (5 nm-1000 nm), and formulated with granules of entactogen to allow kinetic lag.

In certain embodiments, polymer or lipid nanoparticles are used to formulate an SSRI or SNRI for controlled release. In certain embodiments, multilayered nanoparticles are formulated with an entactogen and an SSRI or SNRI to achieve kinetic lag. In certain embodiments, nanoparticles are coated with antigen-recognized antibodies to improve target specificity. In certain embodiments, adjuvants are incorporated to control the release of an SSRI or SNRI from the nanoparticles.

In certain embodiments, an SSRI or SNRI is formulated as solid-lipid nanoparticles (50-1000 nm) composed of lipids that are solids at rt. SLNs of SSRI or SNRI are formulated in solid dosage form with an entactogen, wherein the entactogen is immediately released followed by a kinetic lag, after which the SSRI or SNRI is released as the SLNs biodegrade.

In certain embodiments, natural or synthetic nanofibers are used for controlled release of an SSRI or SNRI. In certain embodiments, swellable or biodegradable polymers are used for modified drug release. In certain embodiments, an entactogen is formulated in highly porous nanofibers to facilitate drug release, and an SSRI or SNRI is formulated in low porosity nanofibers to facilitate modified drug release.

### Particle Size

In certain aspects the entactogen, SSRI, or SNRI is formulated as a microparticle with an average particle size between 10 μm and 700 μm. Modification of particle size can change the rate at which the agent which is a microparticle is released and thus modify the kinetic lag between the entactogen and the SSRI or SNRI compound.

In some embodiments, the average particle size is from 50 μm to 450 μm. In some embodiments, the average particle size of the composition is from 50 μm to 100 μm, 100 μm to 150 μm, 150 μm to 200 μm, 200 μm to 250 μm, 250 μm to 300 μm, 350 μm to 400 μm, 400 μm to 450 μm, 50 μm to 150 μm, 150 μm to 250 μm, 250 μm to 450 μm, 200 μm to 450 μm, 100 μm to 300 μm, 300 μm to 450 μm, or 400 μm to 450 μm.

In some embodiments, the average particle size is from 50 μm to 400 μm. In some embodiments, the average particle size of the composition is from 50 μm to 100 μm, 100 μm to 150 μm, 150 μm to 200 μm, 200 μm to 250 μm, 250 μm to 300 μm, 350 μm to 400 μm, 50 μm to 150 μm, 150 μm to 250 μm, 250 μm to 400 μm, 200 μm to 400 μm, 100 μm to 300 μm, or 200 μm to 400 μm.

In some embodiments, the average particle size is from 400 μm to 450 μm. In some embodiments, the average particle size is 400 μm, 405 μm, 410 μm, 415 μm, 420 μm, 425 μm, 430 μm, 435 μm, 440 μm, 445 μm, or 450 μm.

In some embodiments, the average particle size is from about 415 μm to about 425 μm.

In certain embodiments the pharmaceutical formulation comprises microparticles of MDMA.

### Pharmaceutically Acceptable Salts

The compounds described herein, including enantiomerically enriched mixtures, can be administered if desired as a pharmaceutically acceptable salt or a salt mixture. A salt mixture may be useful to increase solubility of the active substances, to alter pharmacokinetics, or for controlled release or other objective. A salt mixture may comprise 2, 3, 4, 5, 6, or more pharmaceutically acceptable salts together to form a single composition.

The entactogens for use in the present invention are amines and thus basic, and therefore, react with inorganic and organic acids to form pharmaceutically acceptable acid addition salts. In some embodiments, the compounds of the present invention as free amines are oily and have decreased stability at room temperature. In this case it may be beneficial to convert the free amines to their pharmaceutically acceptable acid addition salts for ease of handling and administration because in some embodiments, the pharmaceutically acceptable salt is solid at room temperature.

Acids commonly employed to form such salts are inorganic acids such as hydrochloric acid, hydrobromic acid, hydroiodic acid, sulfuric acid, nitric acid, phosphoric acid, and the like, and organic acids, such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl) benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like. In one embodiment, the compounds of the present invention are administered as an oxalate salt. In one embodiment of the present invention, the compounds are administered as a phosphate salt.

Exemplary salts include, but are not limited to, 2-hydroxyethanesulfonate, 2-naphthalenesulfonate, 3-hydroxy-2-naphthoate, 3-phenylpropionate, acetate, adipate, alginate, amsonate, aspartate, benzenesulfonate, benzoate, besylate, bicarbonate, bisulfate, bitartrate, borate, butyrate, calcium edetate, camphorate, camphorsulfonate, camsylate, carbonate, citrate, clavulariate, cyclopentanepropionate, digluconate, dodecylsulfate, edetate, edisylate, estolate, esylate, ethanesulfonate, finnarate, gluceptate, glucoheptanoate, gluconate, glutamate, glycerophosphate, glycollylarsanilate, hemisulfate, heptanoate, hexafluorophosphate, hexanoate, hexylresorcinate, hydrabamine, hydrobromide, hydrochloride, hydroiodide, hydroxynaphthoate, iodide, sethionate, lactate, lactobionate, laurate, laurylsulphonate, malate, maleate, mandelate, mesylate, methanesulfonate, methylbromide, methylnitrate, methylsulfate, mucate, naphthylate, napsylate, nicotinate, nitrate, N-methylglucamine ammonium salt, oleate, oxalate, palmitate, pamoate, pantothenate, pectinate, persulfate, phosphate, phosphateldiphosphate, picrate, pivalate, polygalacturonate, propionate, p-toluenesulfonate, saccharate, salicylate, stearate, subacetate, succinate, sulfate, sulfosaliculate, suramate, tannate, tartrate, teoclate, thiocyanate, tosylate, triethiodide, undecanoate, and valerate salts, and the like.

Alternatively, exemplary salts include 2-hydroxyethanesulfonate, 2-naphthalenesulfonate, 2-napsylate, 3-hydroxy-2-naphthoate, 3-phenylpropionate, 4-acetamidobenzoate, acefyllinate, acetate, aceturate, adipate, alginate, aminosalicylate, ammonium, amsonate, ascorbate, aspartate, benzenesulfonate, benzoate, besylate, bicarbonate, bisulfate, bitartrate, borate, butyrate, calcium edetate, calcium, camphocarbonate, camphorate, camphorsulfonate, camsylate, carbonate, cholate, citrate, clavulariate, cyclopentanepropionate, cypionate, d-aspartate, d-camsylate, d-lactate, decanoate, dichloroacetate, digluconate, dodecylsulfate, edentate, edetate, edisylate, estolate, esylate, ethanesulfonate, ethyl sulfate, fumarate, furate, fusidate, galactarate (mucate), galacturonate, gallate, gentisate, gluceptate, glucoheptanoate, gluconate, glucuronate, glutamate, glutarate, glycerophosphate, glycolate, glycollylarsanilate, hemisulfate, heptanoate (enanthate), heptanoate, hexafluorophosphate, hexanoate, hexylresorcinate, hippurate, hybenzate, hydrabamine, hydrobromide, hydrobromide/bromide, hydrochloride, hydroiodide, hydroxide, hydroxybenzoate, hydroxynaphthoate, iodide, isethionate, isothionate, 1-aspartate, 1-camsylate, 1-lactate, lactate, lactobionate, laurate, laurylsulphonate, lithium, magnesium, malate, maleate, malonate, mandelate, meso-tartrate, mesylate, methanesulfonate, methylbromide, methylnitrate, methylsulfate, mucate, myristate, N-methylglucamine ammonium salt, napadisilate, naphthylate, napsylate, nicotinate, nitrate, octanoate, oleate, orotate, oxalate, p-toluenesulfonate, palmitate, pamoate, pantothenate, pectinate, persulfate, phenylpropionate, phosphate, phosphateldiphosphate, picrate, pivalate, polygalacturonate, potassium, propionate, pyrophosphate, saccharate, salicylate, salicylsulfate, sodium, stearate, subacetate, succinate, sulfate, sulfosaliculate, sulfosalicylate, suramate, tannate, tartrate, teoclate, terephthalate, thiocyanate, thiosalicylate, tosylate, tribrophenate, triethiodide, undecanoate, undecylenate, valerate, valproate, xinafoate, zinc, and the like. (See Berge et al. (1977) “Pharmaceutical Salts,” J. Pharm. Sci. 66:1-19.) Pharmaceutically acceptable salts include those employing a hydrochloride anion.

### EXAMPLES

### Example 1: Example Combinations

| | |
|---|---|
| Agent | PK/PD |

i.

| | |
|---|---|
| MDMA | Immediate release (an oral Tmax of |
| 10-180 mg | about 45 to about 120 minutes, for |
| | example about 45 to about 75 minutes, |
| | or earlier). |
| Citalopram | Delayed release. Starting at about 120 |
| 20-40 mg | minutes to about 360 minutes |

ii.

| | |
|---|---|
| MDAI | Immediate release (an oral Tmax of |
| 20-400 mg | about 45 to about 120 minutes, for |
| | example about 45 to about 75 minutes, |
| | or earlier). |
| Escitalopram | Delayed release. Delayed release. |
| 20-40 mg | Starting at about 120 minutes to about |
| | 360 minutes |

iii.

| | |
|---|---|
| MDMAI | Immediate release (an oral Tmax of |
| 20-400 mg | about 45 to about 120 minutes, for |
| | example about 45 to about 75 minutes, |
| | or earlier). |
| Fluoxetine | Delayed release. Delayed release. |
| 10-80 mg | Starting at about 120 minutes to about |
| | 360 minutes |

iv.

| | |
|---|---|
| MDMA | Immediate release (an oral Tmax of |
| 10-180 mg | about 45 to about 120 minutes, for |
| | example about 45 to about 75 minutes, |
| | or earlier). |
| Paroxetine | Delayed release. Delayed release. |
| 10-60 mg | Starting at about 120 minutes to about |
| | 360 minutes |

v.

| | |
|---|---|
| MDMA | Immediate release (an oral Tmax of |
| 10-180 mg | about 45 to about 120 minutes, for |
| | example about 45 to about 75 minutes, |
| | or earlier). |
| Milnacipran | Delayed release. Starting at about 120 |
| 10-100 mg | minutes to about 360 minutes |

vi.

| | |
|---|---|
| R-6-MBPB, | Immediate release (an oral Tmax of |
| S-6-MBPB, or | about 45 to about 120 minutes, for |
| racemic 6-MBPB | example about 45 to about 75 |
| 10-200 mg | minutes, or earlier). |
| Duloxetine | Delayed release. Starting at about |
| 20-60 mg | 120 minutes to about 360 minutes |

vii.

| | |
|---|---|
| R-MDA, S-MDA, | Immediate release (an oral Tmax of about |
| or racemic MDA | 45 to about 120 minutes, for example |
| 20-200 mg | about 45 to about 75 minutes, or earlier). |
| Citalopram | Delayed release. Starting at about 120 |
| 20-40 mg | minutes to about 360 minutes |

viii.

| | |
|---|---|
| R-MDMAT, | Immediate release (an oral Tmax of |
| S-MDMAT, or | about 45 to about 120 minutes, for |
| racemic MDMAT | example about 45 to about 75 |
| 10-150 mg | minutes, or earlier). |
| Escitalopram | Delayed release. Delayed release. |
| 20-40 mg | Starting at about 120 minutes to |
| | about 360 minutes |

ix.

| | |
|---|---|
| R-BK-MDMA, | Immediate release (an oral Tmax of |
| S-BK-MDMA, or | about 45 to about 120 minutes, for |
| racemic BK-MDMA | example about 45 to about 75 |
| 10-150 mg | minutes, or earlier). |
| Fluoxetine | Delayed release. Delayed release. |
| 20-40 mg | Starting at about 120 minutes to |
| | about 360 minutes |

x.

| | |
|---|---|
| R-BK-MDEA, | Immediate release (an oral Tmax |
| S-BK-MDEA, or | of about 45 to about 120 minutes, |
| racemic BK-MDEA | for example about 45 to about 75 |
| 5-300 mg | minutes, or earlier). |
| Paroxetine | Delayed release. Delayed release. |
| 10-40 mg | Starting at about 120 minutes to |
| | about 360 minutes |

xi.

| | |
|---|---|
| R-5-APB, | Immediate release (an oral Tmax of |
| S-5-APB, or | about 45 to about 120 minutes, for |
| racemic 5-APB | example about 45 to about 75 |
| 20-160 mg | minutes, or earlier). |
| Milnacipran | Delayed release. Starting at about |
| 10-100 mg | 120 minutes to about 360 minutes |

xii.

| | |
|---|---|
| R-MDA, S-MDA, | Immediate release (an oral Tmax of |
| or racemic MDA | about 45 to about 120 minutes, for |
| 20-200 mg | example about 45 to about 75 minutes, |
| | or earlier). |
| Duloxetine | Delayed release. Starting at about 120 |
| 20-60 mg | minutes to about 360 minutes |

xiii.

| | |
|---|---|
| R-6-APB, | Immediate release (an oral Tmax of |
| S-6-APB, or | about 45 to about 120 minutes, for |
| racemic 6-APB | example about 45 to about 75 |
| 20-160 mg | minutes, or earlier). |
| Citalopram | Delayed release. Starting at about |
| 20-40 mg | 120 minutes to about 360 minutes |

xiv.

| | |
|---|---|
| R-5-MAPB, | Immediate release (an oral Tmax of |
| S-5-MAPB, or | about 45 to about 120 minutes, for |
| racemic 5-MAPB | example about 45 to about 75 |
| 20-160 mg | minutes, or earlier). |
| Escitalopram | Delayed release. Delayed release. |
| 20-40 mg | Starting at about 120 minutes to |
| | about 360 minutes |

xv.

| | |
|---|---|
| R-6-MAPB, | Immediate release (an oral Tmax of |
| S-6-MAPB, or | about 45 to about 120 minutes, for |
| racemic 6-MAPB | example about 45 to about 75 |
| 20-160 mg | minutes, or earlier). |
| Fluoxetine | Delayed release. Delayed release. |
| 20-40 mg | Starting at about 120 minutes to |
| | about 360 minutes |

xvi.

| | |
|---|---|
| R-MDE, S-MDE, | Immediate release (an oral Tmax of |
| or racemic MDE | about 45 to about 120 minutes, for |
| 30-200 mg | example about 45 to about 75 minutes, |
| | or earlier). |
| Paroxetine | Delayed release. Delayed release. |
| 10-60 mg | Starting at about 120 minutes to about |
| | 360 minutes |

xvii.

| | |
|---|---|
| R-6-MAPB, | Immediate release (an oral Tmax of |
| S-6-MAPB, or | about 45 to about 120 minutes, for |
| racemic 6-MAPB | example about 45 to about 75 |
| 20-160 mg | minutes, or earlier). |
| Milnacipran | Delayed release. Starting at about |
| 10-100 mg | 120 minutes to about 360 minutes |

xviii.

| | |
|---|---|
| R-5-MBPB, | Immediate release (an oral Tmax of |
| S-5-MBPB, or | about 45 to about 120 minutes, for |
| racemic 5-MBPB | example about 45 to about 75 |
| 10-200 mg | minutes, or earlier). |
| Duloxetine | Delayed release. Starting at about |
| 20-60 mg | 120 minutes to about 360 minutes |

xix.

| | |
|---|---|
| R-BK-MDMA, | Immediate release (an oral Tmax of |
| S-BK-MDMA, or | about 45 to about 120 minutes, for |
| racemic BK-MDMA | example about 45 to about 75 |
| 10-150 mg | minutes, or earlier). |
| Citalopram | Delayed release. Starting at about |
| 20-40 mg | 120 minutes to about 360 minutes |

xx.

| | |
|---|---|
| R-MBDB, | Immediate release (an oral Tmax of |
| S-MBDB, or | about 45 to about 120 minutes, for |
| racemic MBDB | example about 45 to about 75 minutes, |
| 10-150 mg | or earlier). |
| Escitalopram | Delayed release. Delayed release. |
| 20-40 mg | Starting at about 120 minutes to about |
| | 360 minutes |

xxi.

| | |
|---|---|
| R-5-APB, | Immediate release (an oral Tmax of |
| S-5-APB, or | about 45 to about 120 minutes, for |
| racemic 5-APB | example about 45 to about 75 minutes, |
| 5-100 mg | or earlier). |
| Fluoxetine | Delayed release. Delayed release. |
| 20-40 mg | Starting at about 120 minutes to about |
| | 360 minutes |

xxii.

| | |
|---|---|
| R-BK-MDEA, | Immediate release (an oral Tmax of |
| S-BK-MDEA, or | about 45 to about 120 minutes, for |
| racemic BK-MDEA | example about 45 to about 75 |
| 10-150 mg | minutes, or earlier). |
| Paroxetine | Delayed release. Delayed release. |
| 10-40 mg | Starting at about 120 minutes to |
| | about 360 minutes |

xxiii.

| | |
|---|---|
| R-6-APB, | Immediate release (an oral Tmax of |
| S-6-APB, or | about 45 to about 120 minutes, for |
| racemic 6-APB | example about 45 to about 75 minutes, |
| 5-100 mg | or earlier). |
| Milnacipran | Delayed release. Starting at about 120 |
| 10-100 mg | minutes to about 360 minutes |

xxiv.

| | |
|---|---|
| R-MDA, S-MDA, or | Immediate release (an oral Tmax of |
| racemic MDA | about 45 to about 120 minutes, for |
| 10-150 mg | example about 45 to about 75 minutes, |
| | or earlier). |
| Duloxetine | Delayed release. Starting at about 120 |
| 20-60 mg | minutes to about 360 minutes |

xxv.

| | |
|---|---|
| R-BDB, S-BDB, or | Immediate release (an oral Tmax of |
| racemic BDB | about 45 to about 120 minutes, for |
| 10-150 mg | example about 45 to about 75 |
| | minutes, or earlier). |
| Citalopram | Delayed release. Starting at about |
| 20-40 mg | 120 minutes to about 360 minutes |

xxvi.

| | |
|---|---|
| R-MDE, S-MDE, or | Immediate release (an oral Tmax of |
| racemic MDE | about 45 to about 120 minutes, for |
| 30-200 mg | example about 45 to about 75 minutes, |
| | or earlier). |
| Escitalopram | Delayed release. Delayed release. |
| 20-40 mg | Starting at about 120 minutes to about |
| | 360 minutes |

xxvii.

| | |
|---|---|
| R-BDB, S-BDB, or | Immediate release (an oral Tmax of about |
| racemic BDB | 45 to about 120 minutes, for example about |
| 10-150 mg | 45 to about 75 minutes, or earlier). |
| Fluoxetine | Delayed release. Delayed release. Starting |
| 20-40 mg | at about 120 minutes to about 360 minutes |

xxviii.

| | |
|---|---|
| MDMA | Immediate release (an oral Tmax of about |
| 10-125 mg | 45 to about 120 minutes, for example |
| | about 45 to about 75 minutes, or earlier). |
| Paroxetine | Delayed release. Delayed release. Starting |
| 10-40 mg | at about 120 minutes to about 360 minutes |

xxix.

| | |
|---|---|
| MDMAI | Immediate release (an oral Tmax of |
| 50-300 mg | about 45 to about 120 minutes, for |
| | example about 45 to about 75 minutes, |
| | or earlier). |
| Milnacipran | Delayed release. Starting at about 120 |
| 10-100 mg | minutes to about 360 minutes |

xxx.

| | |
|---|---|
| R-6-APB, | Immediate release (an oral Tmax of |
| S-6-APB, or | about 45 to about 120 minutes, for |
| racemic 6-APB | example about 45 to about 75 minutes, |
| 10-150 mg | or earlier). |
| Duloxetine | Delayed release. Starting at about 120 |
| 20-60 mg | minutes to about 360 minutes |

xxxi.

| | |
|---|---|
| R-BK-5-MAPB, | Immediate release (an oral Tmax of |
| S-BK-5-MAPB, | about 45 to about 120 minutes, for |
| or racemic | example about 45 to about 75 minutes, |
| BK-5-MAPB | or earlier). |
| 5-200 mg | |
| Milnacipran | Delayed release. Starting at about 120 |
| 10-100 mg | minutes to about 360 minutes |

xxxii.

| | |
|---|---|
| R-BK-5-EAPB, | Immediate release (an oral Tmax of |
| S-BK-5-EAPB, | about 45 to about 120 minutes, for |
| or racemic | example about 45 to about 75 minutes, |
| BK-5-EAPB | or earlier). |
| 5-200 mg | |
| Duloxetine | Delayed release. Starting at about 120 |
| 20-60 mg | minutes to about 360 minutes |

xxxiii.

| | |
|---|---|
| R-BK-5-MAPB, | Immediate release (an oral Tmax of |
| S-BK-5-MAPB, | about 45 to about 120 minutes, for |
| or racemic | example about 45 to about 75 |
| BK-5-MAPB | minutes, or earlier). |
| 5-200 mg | |
| Citalopram | Delayed release. Starting at about |
| 20-40 mg | 120 minutes to about 360 minutes |

xxxiv.

| | |
|---|---|
| R-BK-5-EAPB, | Immediate release (an oral Tmax of |
| S-BK-5-EAPB, | about 45 to about 120 minutes, for |
| or racemic | example about 45 to about 75 minutes, |
| BK-5-EAPB | or earlier). |
| 5-200 mg | |
| Escitalopram | Delayed release. Delayed release. |
| 20-40 mg | Starting at about 120 minutes to about |
| | 360 minutes |

### Example 2: Fixed Dose Combinations

A fixed dose combination tablet is formulated to contain an entactogen and a SSRI or SNRI. The tablet is formulated and manufactured in order to achieve a complex modified release profile consisting of immediate release entactogen, then followed by the delayed release of SSRI or SNRI.

### Step 2.1: Agent 1 Granules

A 1 kg granular blend of an entactogen with excipients is prepared as follows:
1. According to the composition and quantities listed in Table 2.1, the active ingredient and excipients (excluding the lubricant) are blended in a V-shell blender for about 10 minutes at about 25 rpm.
2. The lubricant is added to the blender and blending is continued for an additional about 2 minutes at about 25 rpm.
3. The blend is granulated using a roller compactor and oscillating granule mill with a mill screen size of 1.5 mm.
4. The resulting granules are tested and confirmed to have the desired potency of the entactogen.

TABLE 2.1
composition of entactogen granules

| | | Quantity | Quantity, | Quantity/ |
|---|---|---|---|---|
| Component | Function | % | g | tablet, mg |
| Entactogen | active | 30 | 300 | 30 |
| Filler for example | filler | 63.8 | 638 | 63.8 |
| Microcrystalline | | | | |
| cellulose^{a} | | | | |
| Disintegrant | disintegrant | 5 | 50 | 5 |
| for example | | | | |
| Croscarmellose | | | | |
| sodium | | | | |
| Lubricant for example | lubricant | 1.2 | 12 | 1.2 |
| Magnesium stearate | | | | |
| Totals | | 100% | 1,000 g | 100 mg |

### Step 2.2: SSRI or SNRI Enteric Beads

A 2.5 kg granular blend of a SSRI or SNRI with excipients is prepared as follows:
1. According to the composition and quantities listed in Table 2.2, the SSRI or SNRI and enteric coating system are dissolved/dispersed in water to a combined concentration of about 20% w/w.
2. The dispersion is spray coated onto sugar spheres in a fluid bed coater operated in Wurster coating mode, and dried in place, yielding beads coated with active drug and enteric protective polymer materials.
3. The resulting beads are tested to confirm acid resistance and confirmed to have the desired potency. The material is temporarily stored in a sealed LDPE bag and labeled as SSRI or SNRI enteric beads.

TABLE 2.2
composition of SSRI or SNRI enteric granules

| | | Quantity | Quantity, | Quantity/ |
|---|---|---|---|---|
| Component | Function | % | g | tablet, mg |
| SSRI or SNRI | Active | 10 | 250 | 25 |
| Coating for example | enteric polymer | 40 | 1,000 | 100 |
| Eudragit FL 30-D^{a} | coating | | | |
| Carrier for example | carrier particles | 50 | 1,250 | 125 |
| Sugar spheres | | | | |
| Totals | | 100% | 2,500 g | 250 mg |

### Step 2.3: Final Blending and Tablet Compression

A 5 kg final blend and tablets are prepared as follows. Note that the quantities given are the theoretical amounts. To accommodate normal yield losses during processing, the actual mass ratios are maintained per the composition tables, with potency adjustments to ensure the stated drug contents remain correct regardless of batch yield:
1. According to the composition and quantities listed in Table 2.3, the three phases containing active ingredients from Steps 2.1, 2.2, as well as the other listed excipients (excluding the lubricant) are blended in a V-shell blender for about 5 minutes at about 25 rpm.
2. The lubricant is added to the blender and blending is continued for an additional about 2 minutes at about 25 rpm.
3. The resulting final blend is tested for blend uniformity to confirm potencies of the active agents.
4. The final blend is transferred to a rotary tablet press and compressed to form tablets containing the entactogen and the SSRI or SNRI.

TABLE 2.3
composition of tablets

| | | Quantity | Quantity, | Quantity/ |
|---|---|---|---|---|
| Component | Function | % | g | tablet, mg |
| Entactogen | Active | 30 | 300 | 30 |
| SSRI or SNRI | Active | 50 | 2,500 | 250 |
| Filler for example | Filler | 35.5 | 2000 | 200 |
| Microcrystalline | | | | |
| cellulose | | | | |
| Disintegrant | disintegrant | 1.5 | 150 | 15 |
| for example | | | | |
| Croscarmellose | | | | |
| sodium | | | | |
| Lubricant for example | lubricant | 0.5 | 50 | 5 |
| Magnesium stearate | | | | |
| Totals | | 100% | 10,000 g | 1,000 mg |

### Example 3: Fixed Dose Combination Tablets of 30 mg MDMA/25 mg Citalopram

A fixed dose combination tablet is formulated to contain 30 mg of MDMA (as HCl salt). The tablet is formulated and manufactured in order to achieve a modified release profile consisting of immediate release MDMA·HCl, followed by a delayed release of 25 mg citalopram (for example release of citalopram about 3 hours after dosing). The product is formulated and processed in a manner to achieve the stated objectives of the release characteristics for the majority, but not the entirety, of the release timing of each active ingredient.

### Step 3.1: MDMA Granules

A 1 kg granular blend of MDMA·HCl with excipients is prepared as follows:
1. According to the composition and quantities listed in Table 3.1, the active ingredient and excipients (excluding the lubricant) are blended in a V-shell blender for about 10 minutes at about 25 rpm.
2. The lubricant is added to the blender and blending is continued for an additional about 2 minutes at about 25 rpm.
3. The blend is granulated using a roller compactor and oscillating granule mill with a mill screen size of 1.25 mm.
4. The resulting granules are tested and confirmed to have a potency of about 30 mg of MDMA per 100 mg of granular sample material. The material is temporarily stored in a sealed LDPE bag and labeled as MDMA granules.

TABLE 3.1
composition of MDMA granules

| | | Quantity | Quantity, | Quantity/ |
|---|---|---|---|---|
| Component | Function | % | g | tablet, mg |
| MDMA•HCl | Active | 30 | 300 | 30 |
| Microcrystalline | non-brittle | 63.8 | 638 | 63.8 |
| cellulose^{a} | filler | | | |
| Croscarmellose | super- | 5 | 50 | 5 |
| sodium | disintegrant | | | |
| Magnesium stearate | lubricant | 1.2 | 12 | 1.2 |
| Totals | | 100% | 1,000 g | 100 mg |

^{a}note the actual mass of the active ingredient is increased such that the quantity given corresponds to the content of the parent molecule (excluding the mass contribution of the salt form counterion and any other impurities present). Concomitantly, the mass of microcrystalline cellulose is decreased by the same amount that the active ingredient is increased.

### Step 3.2: Citalopram Enteric Beads

A quantity of 2.5 kg of enteric protected beads of citalopram hydrochloride is prepared as follows:
1. According to the composition and quantities listed in Table 3.2, the active ingredient and enteric coating system are dissolved/dispersed in water to a combined concentration of about 20% w/w.
2. The dispersion is spray coated onto sugar spheres in a fluid bed coater operated in Wurster coating mode, and dried in place, yielding beads coated with active drug and enteric protective polymer materials.
3. The resulting beads are tested to confirm acid resistance and confirmed to have a potency of about 25 mg of citalopram (parent compound) per 250 mg of beads. The material is temporarily stored in a sealed LDPE bag and labeled as citalopram enteric beads.

TABLE 3.2
composition of citalopram enteric granules

| | | Quantity | Quantity, | Quantity/ |
|---|---|---|---|---|
| Component | Function | % | g | tablet, mg |
| Citalopram•HCl | Active | 10 | 250 | 25 |
| Eudragit FL 30-D^{a} | enteric polymer | 40 | 1,000 | 100 |
| | coating | | | |
| Sugar spheres | carrier particles | 50 | 1,250 | 125 |
| Totals | | 100% | 2,500 g | 250 mg |

^{a}note the actual mass of the active ingredient is increased such that the quantity given corresponds to the content of the parent molecule (excluding the mass contribution of the salt form counterion and any other impurities present). Concomitantly, the mass of Eudragit is decreased by the same amount that the active ingredient is increased.

### Step 3.3: Final Blending and Tablet Compression

A 5 kg final blend and tablets are prepared as follows. Note that the quantities given are the theoretical amounts. To accommodate normal yield losses during processing, the actual mass ratios are maintained per the composition tables, with potency adjustments to ensure the stated drug contents remain correct regardless of batch yield:
1. According to the composition and quantities listed in Table 3.3, the three phases containing active ingredients from Steps 3.1, and 3.2, as well as the other listed excipients (excluding the lubricant) are blended in a V-shell blender for about 5 minutes at about 25 rpm.
2. The lubricant is added to the blender and blending is continued for an additional about 2 minutes at about 25 rpm.
3. The resulting final blend is tested for blend uniformity to confirm potencies of about 30 mg of MDMA (parent compound), and about 25 mg of citalopram (parent compound): each per 500 mg of final blend.
4. The final blend is transferred to a rotary tablet press and compressed to form tablets containing 30/25 mg respectively of the parent compounds MDMA, and citalopram.

TABLE 3.3
composition of tablets

| | | Quantity | Quantity, | Quantity/ |
|---|---|---|---|---|
| Component | Function | % | g | tablet, mg |
| MDMA•HCl | Active | 30 | 300 | 30 |
| Citalopram enteric | Active | 50 | 2,500 | 250 |
| beads | | | | |
| Microcrystalline | non-brittle | 35.5 | 2000 | 200 |
| cellulose | filler | | | |
| Croscarmellose | super- | 1.5 | 150 | 15 |
| sodium | disintegrant | | | |
| Magnesium stearate | lubricant | 0.5 | 50 | 5 |
| Totals | | 100% | 5,000 g | 500 mg |

### Example 4: Fixed Dose Combination Tablets of 40/30 mg 5-MAPB, Fluoxetine

A fixed dose combination tablet is formulated to contain 40 mg of 5-MAPB (as HCl salt), and 30 mg of fluoxetine (as HCl salt). The tablet is formulated and manufactured to achieve a modified release profile consisting of immediate release 5-MAPB, followed by controlled release of fluoxetine·HCl (for example release of fluoxetine·HCl about 120 minutes after dosing, retarding of the majority of the release onset compared to 5-MAPB). The product is formulated and processed in a manner to achieve the stated objectives of the release characteristics for the majority, but not the entirety, of the release timing of each active ingredient.

### Step 4.1: 5-MAPB Granules

A 1 kg granular blend of 5-MAPB·HCl with excipients is prepared as follows:
1. According to the composition and quantities listed in Table 4.1, the active ingredient and excipients (excluding the lubricant) are blended in a V-shell blender for about 10 minutes at about 25 rpm.
2. The lubricant is added to the blender and blending is continued for an additional about 2 minutes at about 25 rpm.
3. The blend is granulated using a roller compactor and oscillating granule mill with a mill screen size of 1.25 mm.
4. The resulting granules are tested and confirmed to have a potency of about 40 mg of 5-MAPB per 100 mg of granular sample material. The material is temporarily stored in a sealed LDPE bag and labeled as 5-MAPB granules.

TABLE 4.1
composition of 5-MAPB granules

| | | Quantity | Quantity, | Quantity/ |
|---|---|---|---|---|
| Component | Function | % | g | tablet, mg |
| 5-MAPB•HCl | Active | 40 | 400 | 40 |
| Microcrystalline | non-brittle | 55 | 550 | 55 |
| cellulose^{a} | filler | | | |
| Croscarmellose | super- | 4 | 40 | 4 |
| sodium | disintegrant | | | |
| Magnesium stearate | lubricant | 1 | 10 | 1 |
| Totals | | 100% | 1,000 g | 100 mg |

^{a}note the actual mass of the active ingredient is increased such that the quantity given corresponds to the content of the parent molecule (excluding the mass contribution of the salt form counterion and any other impurities present). Concomitantly, the mass of microcrystalline cellulose is decreased by the same amount that the active ingredient is increased.

### Step 4.2: Fluoxetine Granules

A 1.0 kg granular blend of fluoxetine·HCl with excipients is prepared as follows:
1. According to the composition and quantities listed in Table 4.2, fluoxetine and excipients (excluding the lubricant) are blended in a V-shell blender for about 10 minutes at about 25 rpm.
2. The lubricant is added to the blender and blending is continued for an additional about 2 minutes at about 25 rpm.
3. The blend is granulated using a roller compactor and oscillating granule mill with a mill screen size of 1.5 mm.
4. The resulting granules are tested and confirmed to have a potency of about 30 mg of fluoxetine (parent compound) per 100 mg of granular sample material. The material is temporarily stored in a sealed LDPE bag and labeled as fluoxetine granules.

TABLE 4.2
composition of fluoxetine granules

| | | Quantity | Quantity, | Quantity/ |
|---|---|---|---|---|
| Component | Function | % | g | tablet, mg |
| Fluoxetine•HCl | Active | 30 | 300 | 30 |
| Microcrystalline | non-brittle | 65 | 650 | 65 |
| cellulose^{a} | filler | | | |
| Sodium starch | super- | 3.5 | 35 | 3.5 |
| glycolate | disintegrant | | | |
| Magnesium stearate | lubricant | 1.5 | 15 | 1.5 |
| Totals | | 100% | 1,000 g | 100 mg |

^{a}note the actual mass of the active ingredient is increased such that the quantity given corresponds to the content of the parent molecule (excluding the mass contribution of the salt form counterion and any other impurities present). Concomitantly, the mass of microcrystalline cellulose is decreased by the same amount that the active ingredient is increased.

### Step 4.3: Bilayer Tablet Compression

A 2 kg batch of bilayer tablets are prepared as follows. Note that the quantities given are the theoretical amounts. To accommodate normal yield losses during processing, the actual mass ratios are maintained per the composition tables, with potency adjustments to ensure the stated drug contents remain correct regardless of batch yield:
1. According to the composition and quantities listed in Table 4.3, the two phases containing active ingredients from Steps 4.1, and 4.2, are loaded to the individual feed hoppers of a bilayer rotary tablet press.
2. The press is set up such that one layer of the tablet contains 100 mg of the 5-MAPB granules and 100 mg of the fluoxetine granules. These layer weights targets are approximate and controlled within pharmaceutically acceptable ranges of variability.
3. The tablet press is operated to produce bilayer tablets of 200 mg total core weight, containing 40 mg of 5-MAPB, and 30 mg of fluoxetine (parent compounds regardless of salt form or salt content) 4. The core tablets are coated with a cosmetic (non-functional) coating to a weight gain of about 4% w/w using an aqueous spray, fully perforated, pan film coater. The coating material is an Opadry II Swedish Orange coating system (available from Colorcon) containing HPMC polymer, PEG, talc, and inorganic oxide pigments to achieve opacity and colored appearance.
5. After the film coating weight target is achieved within an acceptable range of variability, the spray system is stopped, and the drying system is continued with periodic slow movement of the coating pan for an additional 10-20 minutes to remove excess water from the tablets. When the coated tablets reach an acceptable residual moisture content determined by at-line testing with a loss on drying moisture balance, the coating process is stopped, and the coated tablets are discharged from the coating machine.

TABLE 4.3
composition of tablets

| | | Quantity | Quantity, | | Quantity/ | |
|---|---|---|---|---|---|---|
| Component | Function | % | g | | tablet, mg | |
| 5-MAPB granules | Active | 48 | 1,000 | g | 100 | mg |
| Fluoxetine granules | Active | 48 | 1,000 | g | 100 | mg |
| Opadry II Swedish | Cosmetic | 4 | 21 | g | 2.1 | mg |
| Orange film | coating | | | | | |
| coating | | | | | | |
| Totals | | 100% | 2,021 | g | 202.1 | mg |

### Example 5: Fixed Dose Combination Tablets of 80 mg S-BK-MDMA/40 mg Milnacipran

A fixed dose combination tablet is formulated to contain 80 mg of S-BK-MDMA (as HCl salt). The tablet is formulated and manufactured in order to achieve a modified release profile consisting of immediate release S-BK-MDMA·HCl, followed by a delayed release of 40 mg milnacipran (for example release of milnacipran about 4 hours after dosing). The product is formulated and processed in a manner to achieve the stated objectives of the release characteristics for the majority, but not the entirety, of the release timing of each active ingredient.

### Step 5.1: S-BK-MDMA Granules

A 2.5 kg granular blend of S-BK-MDMA·HCl with excipients is prepared as follows:
1. According to the composition and quantities listed in Table 5.1, the active ingredient and excipients (excluding the lubricant) are blended in a V-shell blender for about 10 minutes at about 25 rpm.
2. The lubricant is added to the blender and blending is continued for an additional about 2 minutes at about 25 rpm.
3. The blend is granulated using a roller compactor and oscillating granule mill with a mill screen size of 1.25 mm.
4. The resulting granules are tested and confirmed to have a potency of about 80 mg of S-BK-MDMA per 250 mg of granular sample material. The material is temporarily stored in a sealed LDPE bag and labeled as S-BK-MDMA granules.

TABLE 5.1
composition of S-BK-MDMA granules

| | | Quantity | Quantity, | Quantity/ |
|---|---|---|---|---|
| Component | Function | % | g | tablet, mg |
| S-BK-MDMA•HCl | Active | 32 | 800 | 80 |
| Microcrystalline | non-brittle | 61.8 | 1545 | 154.5 |
| cellulose^{a} | filler | | | |
| Croscarmellose | super- | 5 | 125 | 12.5 |
| sodium | disintegrant | | | |
| Magnesium stearate | lubricant | 1.2 | 30 | 3 |
| Totals | | 100% | 2,500 g | 250 mg |

^{a}note the actual mass of the active ingredient is increased such that the quantity given corresponds to the content of the parent molecule (excluding the mass contribution of the salt form counterion and any other impurities present). Concomitantly, the mass of microcrystalline cellulose is decreased by the same amount that the active ingredient is increased.

### Step 5.2: Milnacipran Enteric Beads

A quantity of 2.5 kg of enteric protected beads of milnacipran hydrochloride is prepared as follows:
1. According to the composition and quantities listed in Table 5.2, the active ingredient and enteric coating system are dissolved/dispersed in water to a combined concentration of about 20% w/w.
2. The dispersion is spray coated onto sugar spheres in a fluid bed coater operated in Wurster coating mode, and dried in place, yielding beads coated with active drug and enteric protective polymer materials.
3. The resulting beads are tested to confirm acid resistance and confirmed to have a potency of about 40 mg of milnacipran (parent compound) per 250 mg of beads. The material is temporarily stored in a sealed LDPE bag and labeled as milnacipran enteric beads.

TABLE 5.2
composition of milnacipran enteric granules

| | | Quantity | Quantity, | Quantity/ |
|---|---|---|---|---|
| Component | Function | % | g | tablet, mg |
| Milnacipran•HCl | Active | 16 | 400 | 40 |
| Eudragit FL 30-D^{a} | enteric polymer | 36 | 900 | 90 |
| | coating | | | |
| Sugar spheres | carrier particles | 48 | 1,200 | 120 |
| Totals | | 100% | 2,500 g | 250 mg |

^{a}note the actual mass of the active ingredient is increased such that the quantity given corresponds to the content of the parent molecule (excluding the mass contribution of the salt form counterion and any other impurities present). Concomitantly, the mass of Eudragit is decreased by the same amount that the active ingredient is increased.

### Step 5.3: Final Blending and Tablet Compression

A 7.5 kg final blend and tablets are prepared as follows. Note that the quantities given are the theoretical amounts. To accommodate normal yield losses during processing, the actual mass ratios are maintained per the composition tables, with potency adjustments to ensure the stated drug contents remain correct regardless of batch yield:
1. According to the composition and quantities listed in Table 5.3, the three phases containing active ingredients from Steps 5.1, and 5.2, as well as the other listed excipients (excluding the lubricant) are blended in a V-shell blender for about 5 minutes at about 25 rpm.
2. The lubricant is added to the blender and blending is continued for an additional about 2 minutes at about 25 rpm.
3. The resulting final blend is tested for blend uniformity to confirm potencies of about 80 mg of S-BK-MDMA (parent compound), and about 40 mg of milnacipran (parent compound): each per 750 mg of final blend.
4. The final blend is transferred to a rotary tablet press and compressed to form tablets containing 80/40 mg respectively of the parent compounds S-BK-MDMA, and milnacipran.

TABLE 5.3
composition of tablets

| | | Quantity | Quantity, | Quantity/ |
|---|---|---|---|---|
| Component | Function | % | g | tablet, mg |
| S-BK-MDMA•HCl | Active | 33.3 | 2500 | 250 |
| milnacipran enteric | Active | 33.3 | 2500 | 250 |
| beads | | | | |
| Microcrystalline | non-brittle | 31.3 | 2350 | 235 |
| cellulose | filler | | | |
| Croscarmellose | super- | 1.5 | 150 | 15 |
| sodium | disintegrant | | | |
| Magnesium stearate | lubricant | 0.5 | 50 | 5 |
| Totals | | 100% | 7,500 g | 750 mg |

### Example 6: Fixed Dose Combination Tablets of 40 mg 6-APB/20 mg Paroxetine

A fixed dose combination tablet is formulated to contain 40 mg of 6-APB (as HCl salt). The tablet is formulated and manufactured in order to achieve a modified release profile consisting of immediate release 6-APB·HCl, followed by a delayed release of 20 mg paroxetine (for example release of paroxetine about 4 hours after dosing). The product is formulated and processed in a manner to achieve the stated objectives of the release characteristics for the majority, but not the entirety, of the release timing of each active ingredient.

### Step 6.1: 6-APB Granules

A 1.2 kg granular blend of 6-APB·HCl with excipients is prepared as follows:
1. According to the composition and quantities listed in Table 6.1, the active ingredient and excipients (excluding the lubricant) are blended in a V-shell blender for about 10 minutes at about 25 rpm.
2. The lubricant is added to the blender and blending is continued for an additional about 2 minutes at about 25 rpm.
3. The blend is granulated using a roller compactor and oscillating granule mill with a mill screen size of 1.25 mm.
4. The resulting granules are tested and confirmed to have a potency of about 40 mg of 6-APB per 120 mg of granular sample material. The material is temporarily stored in a sealed LDPE bag and labeled as 6-APB granules.

TABLE 6.1
composition of 6-APB granules

| | | Quantity | Quantity, | Quantity/ |
|---|---|---|---|---|
| Component | Function | % | g | tablet, mg |
| 6-APB•HCl | Active | 33.3 | 400 | 40 |
| Microcrystalline | non-brittle | 60.5 | 725.6 | 72.6 |
| cellulose^{a} | filler | | | |
| Croscarmellose | super- | 5 | 60 | 6 |
| sodium | disintegrant | | | |
| Magnesium stearate | lubricant | 1.2 | 14.4 | 1.4 |
| Totals | | 100% | 1200 g | 120 mg |

^{a}note the actual mass of the active ingredient is increased such that the quantity given corresponds to the content of the parent molecule (excluding the mass contribution of the salt form counterion and any other impurities present). Concomitantly, the mass of microcrystalline cellulose is decreased by the same amount that the active ingredient is increased.

### Step 6.2: Paroxetine Enteric Beads

A quantity of 1.2 kg of enteric protected beads of paroxetine hydrochloride is prepared as follows:
1. According to the composition and quantities listed in Table 6.2, the active ingredient and enteric coating system are dissolved/dispersed in water to a combined concentration of about 20% w/w.
2. The dispersion is spray coated onto sugar spheres in a fluid bed coater operated in Wurster coating mode, and dried in place, yielding beads coated with active drug and enteric protective polymer materials.
3. The resulting beads are tested to confirm acid resistance and confirmed to have a potency of about 20 mg of paroxetine (parent compound) per 120 mg of beads. The material is temporarily stored in a sealed LDPE bag and labeled as paroxetine enteric beads.

TABLE 6.2
composition of paroxetine enteric granules

| | | Quantity | Quantity, | Quantity/ |
|---|---|---|---|---|
| Component | Function | % | g | tablet, mg |
| Paroxetine•HCl | Active | 16.7 | 200 | 20 |
| Eudragit FL | enteric polymer | 35.3 | 423.6 | 42.4 |
| 30-D^{a} | coating | | | |
| Sugar spheres | carrier particles | 48 | 576.4 | 57.6 |
| Totals | | 100% | 1200 g | 120 mg |

^{a}note the actual mass of the active ingredient is increased such that the quantity given corresponds to the content of the parent molecule (excluding the mass contribution of the salt form counterion and any other impurities present). Concomitantly, the mass of Eudragit is decreased by the same amount that the active ingredient is increased.

### Step 6.3: Final Blending and Tablet Compression

A 5 kg final blend and tablets are prepared as follows. Note that the quantities given are the theoretical amounts. To accommodate normal yield losses during processing, the actual mass ratios are maintained per the composition tables, with potency adjustments to ensure the stated drug contents remain correct regardless of batch yield:
1. According to the composition and quantities listed in Table 6.3, the three phases containing active ingredients from Steps 6.1, and 6.2, as well as the other listed excipients (excluding the lubricant) are blended in a V-shell blender for about 5 minutes at about 25 rpm.
2. The lubricant is added to the blender and blending is continued for an additional about 2 minutes at about 25 rpm.
3. The resulting final blend is tested for blend uniformity to confirm potencies of about 40 mg of 6-APB (parent compound), and about 20 mg of paroxetine (parent compound): each per 500 mg of final blend.
4. The final blend is transferred to a rotary tablet press and compressed to form tablets containing 40/20 mg respectively of the parent compounds 6-APB, and paroxetine.

TABLE 6.3
composition of tablets

| | | Quantity | Quantity, | Quantity/ |
|---|---|---|---|---|
| Component | Function | % | g | tablet, mg |
| 6-APB•HCl | Active | 24 | 1200 | 120 |
| paroxetine enteric | Active | 24 | 1200 | 120 |
| beads | | | | |
| Microcrystalline | non-brittle | 48 | 2350 | 240 |
| cellulose | filler | | | |
| Croscarmellose | super- | 3.5 | 175 | 17.5 |
| sodium | disintegrant | | | |
| Magnesium stearate | lubricant | 0.5 | 25 | 2.5 |
| Totals | | 100% | 5,000 g | 500 mg |

### Example 7: Fixed Dose Combination Tablets of 60 mg R-MDAT/30 mg Duloxetine

A fixed dose combination tablet is formulated to contain 60 mg of R-MDAT (as HCl salt). The tablet is formulated and manufactured to achieve a modified release profile consisting of immediate release R-MDAT·HCl, followed by a delayed release of 30 mg duloxetine (for example release of duloxetine about 4 hours after dosing). The product is formulated and processed in a manner to achieve the stated objectives of the release characteristics for the majority, but not the entirety, of the release timing of each active ingredient.

### Step 7.1: R-MDAT Granules

A 2 kg granular blend of R-MDAT·HCl with excipients is prepared as follows:
1. According to the composition and quantities listed in Table 7.1, the active ingredient and excipients (excluding the lubricant) are blended in a V-shell blender for about 10 minutes at about 25 rpm.
2. The lubricant is added to the blender and blending is continued for an additional about 2 minutes at about 25 rpm.
3. The blend is granulated using a roller compactor and oscillating granule mill with a mill screen size of 1.25 mm.
4. The resulting granules are tested and confirmed to have a potency of about 60 mg of R-MDAT per 200 mg of granular sample material. The material is temporarily stored in a sealed LDPE bag and labeled as R-MDAT granules.

TABLE 7.1
composition of R-MDAT granules

| | | Quantity | Quantity, | Quantity/ |
|---|---|---|---|---|
| Component | Function | % | g | tablet, mg |
| R-MDAT•HCl | Active | 30 | 600 | 60 |
| Microcrystalline | non-brittle | 64 | 1280 | 128 |
| cellulose^{a} | filler | | | |
| Croscarmellose | super- | 5 | 100 | 10 |
| sodium | disintegrant | | | |
| Magnesium stearate | Lubricant | 1 | 20 | 2 |
| Totals | | 100% | 2000 g | 200 mg |

^{a}note the actual mass of the active ingredient is increased such that the quantity given corresponds to the content of the parent molecule (excluding the mass contribution of the salt form counterion and any other impurities present). Concomitantly, the mass of microcrystalline cellulose is decreased by the same amount that the active ingredient is increased.

### Step 7.2: Duloxetine Enteric Beads

A quantity of 1.5 kg of enteric protected beads of duloxetine hydrochloride is prepared as follows:
1. According to the composition and quantities listed in Table 7.2, the active ingredient and enteric coating system are dissolved/dispersed in water to a combined concentration of about 20% w/w.
2. The dispersion is spray coated onto sugar spheres in a fluid bed coater operated in Wurster coating mode, and dried in place, yielding beads coated with active drug and enteric protective polymer materials.
3. The resulting beads are tested to confirm acid resistance and confirmed to have a potency of about 30 mg of duloxetine (parent compound) per 150 mg of beads. The material is temporarily stored in a sealed LDPE bag and labeled as duloxetine enteric beads.

TABLE 7.2
composition of duloxetine enteric granules

| | | Quantity | Quantity, | Quantity/ |
|---|---|---|---|---|
| Component | Function | % | g | tablet, mg |
| Duloxetine•HCl | Active | 15 | 300 | 30 |
| Eudragit FL | enteric polymer | 37 | 555 | 55.5 |
| 30-D^{a} | coating | | | |
| Sugar spheres | carrier particles | 48 | 645 | 64.5 |
| Totals | | 100% | 1500 g | 150 mg |

^{a}note the actual mass of the active ingredient is increased such that the quantity given corresponds to the content of the parent molecule (excluding the mass contribution of the salt form counterion and any other impurities present). Concomitantly, the mass of Eudragit is decreased by the same amount that the active ingredient is increased.

### Step 7.3: Final Blending and Tablet Compression

A 5 kg final blend and tablets are prepared as follows. Note that the quantities given are the theoretical amounts. To accommodate normal yield losses during processing, the actual mass ratios are maintained per the composition tables, with potency adjustments to ensure the stated drug contents remain correct regardless of batch yield:
1. According to the composition and quantities listed in Table 7.3, the three phases containing active ingredients from Steps 7.1, and 7.2, as well as the other listed excipients (excluding the lubricant) are blended in a V-shell blender for about 5 minutes at about 25 rpm.
2. The lubricant is added to the blender and blending is continued for an additional about 2 minutes at about 25 rpm.
3. The resulting final blend is tested for blend uniformity to confirm potencies of about 60 mg of R-MDAT (parent compound), and about 30 mg of duloxetine (parent compound): each per 500 mg of final blend.
4. The final blend is transferred to a rotary tablet press and compressed to form tablets containing 60/30 mg respectively of the parent compounds R-MDAT, and duloxetine.

TABLE 7.3
composition of tablets

| | | Quantity | Quantity, | Quantity/ |
|---|---|---|---|---|
| Component | Function | % | g | tablet, mg |
| R-MDAT•HCl | Active | 40 | 2000 | 200 |
| duloxetine enteric | Active | 30 | 1500 | 150 |
| beads | | | | |
| Microcrystalline | non-brittle | 27 | 1350 | 135 |
| cellulose | filler | | | |
| Croscarmellose | super- | 2.5 | 125 | 12.5 |
| sodium | disintegrant | | | |
| Magnesium stearate | Lubricant | 0.5 | 25 | 2.5 |
| Totals | | 100% | 5,000 g | 500 mg |

### Example 8: Fixed Dose Combination Tablets of 100/30 mg MDMA, Escitalopram

A fixed dose combination tablet is formulated to contain 100 mg of MDMAI (as HCl salt), and 30 mg of escitalopram (as HCl salt). The tablet is formulated and manufactured to achieve a modified release profile consisting of immediate release MDMAI, followed by controlled release of escitalopram·HCl (for example release of escitalopram·HCl about 120 minutes after dosing, retarding of the majority of the release onset compared to MDMAI). The product is formulated and processed in a manner to achieve the stated objectives of the release characteristics for the majority, but not the entirety, of the release timing of each active ingredient.

### Step 8.1: MDMAI Granules

A 2.5 kg granular blend of MDMAI·HCl with excipients is prepared as follows:
1. According to the composition and quantities listed in Table 8.1, the active ingredient and excipients (excluding the lubricant) are blended in a V-shell blender for about 10 minutes at about 25 rpm.
2. The lubricant is added to the blender and blending is continued for an additional about 2 minutes at about 25 rpm.
3. The blend is granulated using a roller compactor and oscillating granule mill with a mill screen size of 1.25 mm.
4. The resulting granules are tested and confirmed to have a potency of about 100 mg of MDMAI per 250 mg of granular sample material. The material is temporarily stored in a sealed LDPE bag and labeled as MDMAI granules.

TABLE 8.1
composition of MDMAI granules

| | | Quantity | Quantity, | Quantity/ |
|---|---|---|---|---|
| Component | Function | % | g | tablet, mg |
| MDMAI•HCl | Active | 40 | 1000 | 100 |
| Microcrystalline | non-brittle | 55 | 1375 | 137.5 |
| cellulose^{a} | filler | | | |
| Croscarmellose | super- | 4 | 100 | 10 |
| sodium | disintegrant | | | |
| Magnesium stearate | Lubricant | 1 | 25 | 2.5 |
| Totals | | 100% | 2,500 g | 250 mg |

^{a}note the actual mass of the active ingredient is increased such that the quantity given corresponds to the content of the parent molecule (excluding the mass contribution of the salt form counterion and any other impurities present). Concomitantly, the mass of microcrystalline cellulose is decreased by the same amount that the active ingredient is increased.

### Step 8.2: Escitalopram Granules

A 2.5 kg granular blend of escitalopram·HCl with excipients is prepared as follows:
1. According to the composition and quantities listed in Table 8.2, escitalopram and excipients (excluding the lubricant) are blended in a V-shell blender for about 10 minutes at about 25 rpm.
2. The lubricant is added to the blender and blending is continued for an additional about 2 minutes at about 25 rpm.
3. The blend is granulated using a roller compactor and oscillating granule mill with a mill screen size of 1.5 mm.
4. The resulting granules are tested and confirmed to have a potency of about 30 mg of escitalopram (parent compound) per 250 mg of granular sample material. The material is temporarily stored in a sealed LDPE bag and labeled as escitalopram granules.

TABLE 8.2
composition of escitalopram granules

| | | Quantity | Quantity, | Quantity/ |
|---|---|---|---|---|
| Component | Function | % | g | tablet, mg |
| Escitalopram•HCl | Active | 12 | 300 | 30 |
| Microcrystalline | non-brittle | 83 | 2075 | 207.5 |
| cellulose^{a} | filler | | | |
| Sodium starch | super- | 3.5 | 875 | 8.75 |
| glycolate | disintegrant | | | |
| Magnesium stearate | Lubricant | 1.5 | 37.5 | 3.75 |
| Totals | | 100% | 2,500 g | 250 mg |

^{a}note the actual mass of the active ingredient is increased such that the quantity given corresponds to the content of the parent molecule (excluding the mass contribution of the salt form counterion and any other impurities present). Concomitantly, the mass of microcrystalline cellulose is decreased by the same amount that the active ingredient is increased.

### Step 8.3: Bilayer Tablet Compression

A 5 kg batch of bilayer tablets are prepared as follows. Note that the quantities given are the theoretical amounts. To accommodate normal yield losses during processing, the actual mass ratios are maintained per the composition tables, with potency adjustments to ensure the stated drug contents remain correct regardless of batch yield:
1. According to the composition and quantities listed in Table 8.3, the two phases containing active ingredients from Steps 8.1, and 8.2, are loaded to the individual feed hoppers of a bilayer rotary tablet press
2. The press is set up such that one layer of the tablet contains 250 mg of the MDMAI granules and 250 mg of the escitalopram granules. These layer weights targets are approximate and controlled within pharmaceutically acceptable ranges of variability.
3. The tablet press is operated to produce bilayer tablets of 500 mg total core weight, containing 100 mg of MDMAI, and 30 mg of escitalopram (parent compounds regardless of salt form or salt content)
4. The core tablets are coated with a cosmetic (non-functional) coating to a weight gain of about 4% w/w using an aqueous spray, fully perforated, pan film coater. The coating material is an Opadry II Swedish Orange coating system (available from Colorcon) containing HPMC polymer, PEG, talc, and inorganic oxide pigments to achieve opacity and colored appearance.
5. After the film coating weight target is achieved within an acceptable range of variability, the spray system is stopped, and the drying system is continued with periodic slow movement of the coating pan for an additional 10-20 minutes to remove excess water from the tablets. When the coated tablets reach an acceptable residual moisture content determined by at-line testing with a loss on drying moisture balance, the coating process is stopped, and the coated tablets are discharged from the coating machine.

TABLE 8.3
composition of tablets

| | | | | Quantity/ | |
|---|---|---|---|---|---|
| Component | Function | Quantity, g | | tablet, mg | |
| MDMAI granules | Active | 2,500 | g | 250 | mg |
| Escitalopram granules | Active | 2,500 | g | 250 | mg |
| Opadry II Swedish | Cosmetic | 200 | g | 20 | mg |
| Orange film coating | coating | | | | |
| Totals | | 5,200 | g | 520 | mg |

### Example 9: Fixed Dose Combination Tablets of 100/30 mg S-5-MBPB, Paroxetine

A fixed dose combination tablet is formulated to contain 100 mg of S-5-MBPB (as HCl salt), and 30 mg of paroxetine (as HCl salt). The tablet is formulated and manufactured to achieve a modified release profile consisting of immediate release S-5-MBPB, followed by controlled release of paroxetine·HCl (for example release of paroxetine·HCl about 120 minutes after dosing, retarding of the majority of the release onset compared to S-5-MBPB). The product is formulated and processed in a manner to achieve the stated objectives of the release characteristics for the majority, but not the entirety, of the release timing of each active ingredient.

### Step 9.1: S-5-MBPB Granules

A 2.5 kg granular blend of S-5-MBPB·HCl with excipients is prepared as follows:
1. According to the composition and quantities listed in Table 9.1, the active ingredient and excipients (excluding the lubricant) are blended in a V-shell blender for about 10 minutes at about 25 rpm.
2. The lubricant is added to the blender and blending is continued for an additional about 2 minutes at about 25 rpm.
3. The blend is granulated using a roller compactor and oscillating granule mill with a mill screen size of 1.25 mm.
4. The resulting granules are tested and confirmed to have a potency of about 100 mg of S-5-MBPB per 250 mg of granular sample material. The material is temporarily stored in a sealed LDPE bag and labeled as S-5-MBPB granules.

TABLE 9.1
composition of S-5-MBPB granules

| | | Quantity | Quantity, | Quantity/ |
|---|---|---|---|---|
| Component | Function | % | g | tablet, mg |
| S-5-MBPB•HCl | Active | 40 | 1000 | 100 |
| Microcrystalline | non-brittle | 55 | 1375 | 137.5 |
| cellulose^{a} | filler | | | |
| Croscarmellose | super- | 4 | 100 | 10 |
| sodium | disintegrant | | | |
| Magnesium stearate | Lubricant | 1 | 25 | 2.5 |
| Totals | | 100% | 2,500 g | 250 mg |

^{a}note the actual mass of the active ingredient is increased such that the quantity given corresponds to the content of the parent molecule (excluding the mass contribution of the salt form counterion and any other impurities present). Concomitantly, the mass of microcrystalline cellulose is decreased by the same amount that the active ingredient is increased.

### Step 9.2: Paroxetine Solid-Lipid Nanoparticles (SLN)

A 4 kg SLN nanoparticle blend of paroxetine·HCl is prepared using following steps.
1. Kolliwax GMS (lipid) (750 g) is heated at 75° C., 5 to 10° C. above the melting point of the solid lipid. 250 g paroxetine is added to the melted lipid and drug was dissolved in the lipid phase.
2. Aqueous solution of surfactant (3500 g Tween 80) and the lipid are mixed under magnetic stirring at 600 rpm, maintaining the temperature at 75° C., and the stirring is continued at 600 rpm till the room temperature is reached.
3. The untrapped drug is separated from SLNs in the suspension by centrifugation at 14,000 rpm at rt. After removing the supernatant, separated SLNs are washed with distilled water.
4. SLNs are lyophilized to obtain dry SLNs. SLNs are assessed for entrapment efficiency, particle size, PDI, and zeta potential.

### Step 9.3: Bilayer Tablet Compression

A 5 kg batch of bilayer tablets are prepared as follows. Note that the quantities given are the theoretical amounts. To accommodate normal yield losses during processing, the actual mass ratios are maintained per the composition tables, with potency adjustments to ensure the stated drug contents remain correct regardless of batch yield:
1. According to the composition and quantities listed in Table 9.2, the two phases containing active ingredients from Steps 9.1, and 9.2, are loaded to the individual feed hoppers of a bilayer rotary tablet press.
2. The press is set up such that one layer of the tablet contains 250 mg of the S-5-MBPB granules and 250 mg of the paroxetine nanoparticles. These layer weights targets are approximate and controlled within pharmaceutically acceptable ranges of variability.
3. The tablet press is operated to produce bilayer tablets of 500 mg total core weight, containing 100 mg of S-5-MBPB, and 30 mg of paroxetine (parent compounds regardless of salt form or salt content)
4. The core tablets are coated with a cosmetic (non-functional) coating to a weight gain of about 4% w/w using an aqueous spray, fully perforated, pan film coater. The coating material is an Opadry II Swedish Orange coating system (available from Colorcon) containing HPMC polymer, PEG, talc, and inorganic oxide pigments to achieve opacity and colored appearance.
5. After the film coating weight target is achieved within an acceptable range of variability, the spray system is stopped, and the drying system is continued with periodic slow movement of the coating pan for an additional 10-20 minutes to remove excess water from the tablets. When the coated tablets reach an acceptable residual moisture content determined by at-line testing with a loss on drying moisture balance, the coating process is stopped, and the coated tablets are discharged from the coating machine.

TABLE 9.2
composition of tablets

| | | | | Quantity/ | |
|---|---|---|---|---|---|
| Component | Function | Quantity, g | | tablet, mg | |
| S-5-MBPB granules | Active | 2,500 | g | 250 | mg |
| Paroxetine SLN | Active | 2,500 | g | 250 | mg |
| Opadry II Swedish | Cosmetic | 200 | g | 20 | mg |
| Orange film coating | coating | | | | |
| Totals | | 5,200 | g | 520 | mg |

### Example 10: Fixed Dose Combination Capsules of 80 mg R-5-MBPBT/40 mg Duloxetine

A fixed dose combination tablet is formulated to contain 80 mg of R-5-MBPBT (as HCl salt). The tablet is formulated and manufactured in order to achieve a modified release profile consisting of immediate release R-5-MBPBT·HCl, followed by a delayed release of 40 mg duloxetine (for example release of duloxetine about 2 hours after dosing). The product is formulated and processed in a manner to achieve the stated objectives of the release characteristics for the majority, but not the entirety, of the release timing of each active ingredient.

### Step 10.1: R-5-MBPBT Granules

A 2.5 kg granular blend of R-5-MBPBT·HCl with excipients is prepared as follows:
1. According to the composition and quantities listed in Table 10.1, the active ingredient and excipients (excluding the lubricant) are blended in a V-shell blender for about 10 minutes at about 25 rpm.
2. The lubricant is added to the blender and blending is continued for an additional about 2 minutes at about 25 rpm.
3. The blend is granulated using a roller compactor and oscillating granule mill with a mill screen size of 1.25 mm.
4. The resulting granules are tested and confirmed to have a potency of about 80 mg of R-5-MBPBT per 250 mg of granular sample material. The material is temporarily stored in a sealed LDPE bag and labeled as R-5-MBPBT granules.

TABLE 10.1
composition of R-5-MBPBT granules

| | | Quantity | Quantity, | Quantity/ |
|---|---|---|---|---|
| Component | Function | % | g | tablet, mg |
| R-5-MBPBT•HCl | Active | 32 | 800 | 80 |
| Microcrystalline | non-brittle | 61.8 | 1545 | 154.5 |
| cellulose^{a} | filler | | | |
| Croscarmellose | super- | 5 | 125 | 12.5 |
| sodium | disintegrant | | | |
| Magnesium stearate | Lubricant | 1.2 | 30 | 3 |
| Totals | | 100% | 2,500 g | 250 mg |

^{a}note the actual mass of the active ingredient is increased such that the quantity given corresponds to the content of the parent molecule (excluding the mass contribution of the salt form counterion and any other impurities present). Concomitantly, the mass of microcrystalline cellulose is decreased by the same amount that the active ingredient is increased.

### Step 10.2: Duloxetine Enteric Beads

A quantity of 2.5 kg of enteric protected beads of duloxetine hydrochloride is prepared as follows:
1. According to the composition and quantities listed in Table 10.2, the active ingredient and enteric coating system are dissolved/dispersed in water to a combined concentration of about 20% w/w.
2. The dispersion is spray coated onto sugar spheres in a fluid bed coater operated in Wurster coating mode, and dried in place, yielding beads coated with active drug and enteric protective polymer materials.
3. The resulting beads are tested to confirm acid resistance and confirmed to have a potency of about 40 mg of duloxetine (parent compound) per 250 mg of beads. The material is temporarily stored in a sealed LDPE bag and labeled as duloxetine enteric beads.

TABLE 10.2
composition of duloxetine enteric granules

| | | Quantity | Quantity, | Quantity/ |
|---|---|---|---|---|
| Component | Function | % | g | tablet, mg |
| Duloxetine•HCl | Active | 16 | 400 | 40 |
| Eudragit FL | enteric polymer | 36 | 900 | 90 |
| 30-D^{a} | coating | | | |
| Sugar spheres | carrier particles | 48 | 1,200 | 120 |
| Totals | | 100% | 2,500 g | 250 mg |

^{a}note the actual mass of the active ingredient is increased such that the quantity given corresponds to the content of the parent molecule (excluding the mass contribution of the salt form counterion and any other impurities present). Concomitantly, the mass of Eudragit is decreased by the same amount that the active ingredient is increased.

### Step 10.3: Final Blending and Capsule Filling

A 5 kg final blend and tablets are prepared as follows. Note that the quantities given are the theoretical amounts. To accommodate normal yield losses during processing, the actual mass ratios are maintained per the composition tables, with potency adjustments to ensure the stated drug contents remain correct regardless of batch yield:
1. According to the composition and quantities listed in Table 10.3, the three phases containing active ingredients from Steps 10.1, and 10.2, as well as the other listed excipients (excluding the lubricant) are blended in a V-shell blender for about 5 minutes at about 25 rpm.
2. The lubricant is added to the blender and blending is continued for an additional about 2 minutes at about 25 rpm.
3. The resulting final blend is tested for blend uniformity to confirm potencies of about 80 mg of R-5-MBPBT (parent compound), and about 40 mg of duloxetine (parent compound): each per 500 mg of final blend.
4. The final blend is filled into hard or soft gelatin capsules containing 80/40 mg respectively of the parent compounds R-5-MBPBT, and duloxetine.

TABLE 10.3
composition of capsules

| | | Quantity | Quantity, | Quantity/ |
|---|---|---|---|---|
| Component | Function | % | g | tablet, mg |
| R-5-MBPBT•HCl | active | 50 | 2500 | 250 |
| duloxetine enteric beads | active | 50 | 2500 | 250 |
| Magnesium stearate | lubricant | 1 | 50 | 5 |
| Totals | | 100% | 5050 g | 500 mg |

### Example 11: Fixed Dose Combination Capsules of 100/30 mg RS-MBDB, Citalopram

A fixed dose combination tablet is formulated to contain 100 mg of RS-MBDB (as HCl salt), and 30 mg of citalopram (as HCl salt). The tablet is formulated and manufactured to achieve a modified release profile consisting of immediate release RS-MBDB, followed by controlled release of citalopram·HCl (for example release of citalopram·HCl about 120 minutes after dosing, retarding of the majority of the release onset compared to RS-MBDB). The product is formulated and processed in a manner to achieve the stated objectives of the release characteristics for the majority, but not the entirety, of the release timing of each active ingredient.

### Step 11.1: RS-MBDB Granules

A 2.5 kg granular blend of RS-MBDB·HCl with excipients is prepared as follows:
1. According to the composition and quantities listed in Table 11.1, the active ingredient and excipients (excluding the lubricant) are blended in a V-shell blender for about 10 minutes at about 25 rpm.
2. The lubricant is added to the blender and blending is continued for an additional about 2 minutes at about 25 rpm.
3. The blend is granulated using a roller compactor and oscillating granule mill with a mill screen size of 1.25 mm.
4. The resulting granules are tested and confirmed to have a potency of about 100 mg of RS-MBDB per 250 mg of granular sample material. The material is temporarily stored in a sealed LDPE bag and labeled as RS-MBDB granules.

TABLE 11.1
composition of RS-MBDB granules

| | | Quantity | Quantity, | Quantity/ |
|---|---|---|---|---|
| Component | Function | % | g | tablet, mg |
| RS-MBDB•HCl | Active | 40 | 1000 | 100 |
| Microcrystalline | non-brittle | 55 | 1375 | 137.5 |
| cellulose^{a} | filler | | | |
| Croscarmellose | super- | 4 | 100 | 10 |
| sodium | disintegrant | | | |
| Magnesium stearate | Lubricant | 1 | 25 | 2.5 |
| Totals | | 100% | 2,500 g | 250 mg |

^{a}note the actual mass of the active ingredient is increased such that the quantity given corresponds to the content of the parent molecule (excluding the mass contribution of the salt form counterion and any other impurities present). Concomitantly, the mass of microcrystalline cellulose is decreased by the same amount that the active ingredient is increased.

### Step 11.2: Citalopram Solid-Lipid Nanoparticles (SLN)

A 4 kg SLN nanoparticle blend of citalopram·HCl is prepared using following steps.
1. Kolliwax GMS (lipid) (750 g) is heated at 75° C., 5 to 10° C. above the melting point of the solid lipid. 250 g paroxetine is added to the melted lipid and drug was dissolved in the lipid phase.
2. Aqueous solution of surfactant (3500 g Tween 80) and the lipid are mixed under magnetic stirring at 600 rpm, maintaining the temperature at 75° C., and the stirring is continued at 600 rpm till the room temperature is reached.
3. The untrapped drug is separated from SLNs in the suspension by centrifugation at 14,000 rpm at rt. After removing the supernatant, separated SLNs are washed with distilled water.
4. SLNs are lyophilized to obtain dry SLNs. The resulting SLNs are tested to confirm acid resistance and confirmed to have a potency of about 30 mg of citalopram (parent compound) per 250 mg of nanoparticles.

### Step 11.3: Final Blending and Capsule Filling

A 5 kg final blend and capsules are prepared as follows. Note that the quantities given are the theoretical amounts. To accommodate normal yield losses during processing, the actual mass ratios are maintained per the composition tables, with potency adjustments to ensure the stated drug contents remain correct regardless of batch yield:
1. According to the composition and quantities listed in Table 11.3, the two phases containing active ingredients from Steps 11.1, and 11.2 are blended in a V-shell blender for about 5 minutes at about 25 rpm.
2. The lubricant is added to the blender and blending is continued for an additional about 2 minutes at about 25 rpm.
3. The resulting final blend is tested for blend uniformity to confirm potencies of about 100 mg of RS-MBDB (parent compound), and about 30 mg of citalopram (parent compound): each per 500 mg of final blend.
4. The final blend is filled into hard or soft gelatin capsules containing 100/30 mg respectively of the parent compounds RS-MBDB, and citalopram.

TABLE 11.2
composition of capsules

| | | | Quantity/ |
|---|---|---|---|
| Component | Function | Quantity, g | tablet, mg |
| RS-MBDB granules | Active | 2,500 g | 250 mg |
| Citalopram SLN | Active | 2,500 g | 250 mg |
| Magnesium stearate | lubricant | 50 | 5 |
| Totals | | 5,050 g | 505 mg |

### Example 12: Measuring Serotonin and Dopamine Releasing Effects of Molecules

The serotonin releasing effects of molecules may be measured in a number of ways known to those skilled in the arts. These include measuring serotonin increases in plasma, decreased serotonin uptake in cells, serotonin release from rat synaptosomes, and serotonin release from cells that have human monoamine transporters on their surface. These assay methods are briefly detailed below.

### Serotonin Release Assay Methods Using Plasma

Serum serotonin can be measured using High Performance Liquid Chromatography and Fluorescence Detection. Venipuncture collects at least 1 mL of sample, which is spun with serum frozen to below −20° C. within 2 hours of collection. For active compounds, assay results will show increases in serum serotonin, indicating that the compound is a releaser of serotonin.

### Serotonin Release Assay Using Human Serotonin Transporter (SERT, SLC6A4) in HEK-293 Cells

Human recombinant serotonin transporter expressed in HEK-293 cells are plated. Test compound and/or vehicle is preincubated with cells (1×10E5/ml) in modified Tris-HEPES buffer pH 7.1 for 20 minutes at 25° C. and 65 nM. [³H]Serotonin is then added for an additional 15 minute incubation period. Bound cells are filtered and counted to determine [3H]Serotonin uptake. Compounds are screened at concentrations from 10 to 0.001 μM or similar. Reduction of [³H]Serotonin uptake relative to 1 μM fluoxetine indicates inhibitory activity.

### Serotonin Release and Dopamine Release Assay Methods Using Rat Synaptosomes

An alternative, invasive method of measuring compound interactions with the serotonin, dopamine, or norepinephrine transporter can be conducted according to the methods of Solis et al (2017. Neuropsychopharmacology, 42(10), 1950-1961) and Rothman and Baumann (Partilla et al. 2016. In: Bonisch S, Sitte H H (eds) Neurotransmitter Transporters Springer; New York, pp 41-52).

Male Sprague-Dawley rats (Charles River, Kingston, NY, USA) are used for the synaptosome assays. Rats are group-housed with free access to food and water, under a 12 h light/dark cycle with lights on at 0700 h. Rats are euthanized by CO2 narcosis, and synaptosomes prepared from brains using standard procedures (Rothman, R. B., & Baumann, M. H. (2003). Monoamine transporters and psychostimulant drugs. European journal of pharmacology, 479(1-3), 23-40). Transporter uptake and release assays are performed as described previously (Solis et al. 2017. Neuropsychopharmacology, 42(10), 1950-1961). In brief, synaptosomes are prepared from caudate tissue for dopamine transporter (DAT) assays, and from whole brain minus caudate and cerebellum for norepinephrine transporter (NET) and serotonin (5-HT) transporter (SERT) assays.

For uptake inhibition assays, 5 nM [³H]dopamine, [³H]norepinephrine, or [³H]5-HT are used for DAT, NET, or SERT assays respectively. To optimize uptake for a single transporter, unlabeled blockers are included to prevent the uptake of [3H]neurotransmitter by competing transporters. Uptake inhibition is initiated by incubating synaptosomes with various doses of test compound and [³H]neurotransmitter in Krebs-phosphate buffer. Uptake assays were terminated by rapid vacuum filtration and retained radioactivity is quantified with liquid scintillation counting (Baumann et al. 2013. Neuropsychopharmacology, 38(4), 552-562).

For release assays, 9 nM [³H]MPP+ (1-methyl-4-phenylpyridinium) is used as the radiolabeled substrate for DAT and NET, whereas 5 nM [³H]5-HT is used for SERT. Alternatively [³H]dopamine and [³H]norepinephrine may be used for DAT and NET assays, respectively. All buffers used in the release assay contain 1 μM reserpine to block vesicular uptake of substrates. The selectivity of release assays is optimized for a single transporter by including unlabeled blockers to prevent the uptake of [³H]MPP+ or [3H]5-HT by competing transporters. Synaptosomes are preloaded with radiolabeled substrate in Krebs-phosphate buffer for 1 hr to reach steady state. Release assays are initiated by incubating preloaded synaptosomes with various concentrations of the test drug. Release is terminated by vacuum filtration and retained radioactivity quantified by liquid scintillation counting.

Effects of test drugs on release are expressed as a percent of maximal release, with maximal release (i.e., 100% Emax) defined as the release produced by tyramine or a similar releaser (such as dextroamphetamine at DAT and NET and norfenfluramine at SERT) at doses that evoke the efflux of all ‘releasable’ tritium by synaptosomes (10 μM tyramine for DAT and NET assay conditions, and 100 μM tyramine for SERT assay conditions). Effects of test drugs on release are analyzed by nonlinear regression. Dose-response values for the uptake release are fit to the equation, Y(x)=Ymin+(Ymax−Ymin)/(1+10 exp[(log EC₅₀−log x)]×n), where x is the concentration of the compound tested, Y(x) is the response measured, Ymax is the maximal response, EC₅₀ is the concentration that yields half-maximal release, and n is the Hill slope parameter. EC₅₀s for release of less than 10 uM, but often less than 1 uM, are usually considered indicative of substrate-type releasers.

### Serotonin Release Assay Methods Using Human SERT

Chinese hamster ovary cells or similar expressing human SERT are seeded in Cytostar™ (PerkinElmer) plate with standard culture medium the day before the experiment at a single density (5000 cells/assay). Cells are incubated overnight with 5% CO₂ at 37° C. The day of experiment, the medium was replaced by incubation buffer (140 mM NaCl, 4.8 mM KCl, 1.2 mM MgSO₄, 0.1 mM KH2PO₄, 10 mM HEPES, pH 7.4) with a single concentration of [3H]serotonin at 150 nM.

Cells are incubated at room temperature at different incubation times and radioactivity is counted. Test compounds are measured at concentrations of 1e-10, 1e-09, 1e-08, 1e-07, 1e-06, 1e-05, and 1e-04 M or similar.

EC/IC₅₀s can be calculated using the R packages drm (to fit the regression model) and LL.4 (to define the structure of the log-logistic regression model) or similar statistical software. Values are fit to the following function: 10 f(x)=c+(d−c)/(1+exp(b (log(x)−log(e))) where b=the Hill coefficient, c=minimum value, d=maximum value, and e=EC₅₀/IC₅₀.

### Dopamine-Release Assay Methods Using Human DAT

Chinese hamster ovary cells or similar expressing human DAT are seeded in Cytostar™ plate with standard culture medium the day before experiment at one single density (2500 cells/assay). Cells are incubated overnight with 5% CO₂ at 37° C. The day of experiment, the medium is replaced by incubation buffer (TrisHCl 5 mM, 120 mM NaCl, 5.4 mM KCl, 1.2 mM MgSO₄, 1.2 mM CaCl₂), Glucose 5 mM, 7.5 mM HEPES, pH 7.4) with a single concentration of [³H]dopamine at 300 nM. In control wells, the specificity of DAT uptake is verified by adding the reference control GBR 12909 (10 μM). For all assays, three reference conditions are employed: (1) radioligand-containing buffer only, to verify the control level of release, (2) buffer with 1% DMSO (solvent used to solubilize the test compounds), (3) 100 μM amphetamine (in 1% DMSO) to make it possible to calculate a relative Eₘₐₓ.

Cells are incubated at room temperature at different incubation times and radioactivity is counted. Test compounds are measured at concentrations of 1e-10, 1e-09, 1e-08, 1e-07, 1e-06, 1e-05, and 1e-04 M or similar.

EC/IC₅₀s can be calculated using the R packages drm (to fit the regression model) and LL.4 (to define the structure of the log-logistic regression model) or similar statistical software. Values are fit to the following function: 10 f(x)=c+(d−c)/(1+exp(b (log(x)−log(e))) where b=the Hill coefficient, c=minimum value, d=maximum value, and e=EC₅₀/IC₅₀.

### Example 13: Evaluation of Therapeutic Profile of Entactogens in Humans

The methods and compositions disclosed herein can be evaluated by measuring desirable and undesirable effects of entactogens and showing that the ratio of the two is increased. Desirable effects of an entactogen include decreased neuroticism, increased authenticity, and positive mood (often including feelings of elation, emotional closeness to others, and love). Additionally, decreased changes in blood pressure and heart rate are also desirable.

If the entactogen is administered to a person with some mental health condition, then standard measures for assessing the severity of that condition may also be used to show therapeutic benefits. For example, the Clinician-Administered PTSD Scale for DSM-5 (CAPS-5) may be used to measure therapeutic benefits in an individual with PTSD.

The entactogenic effect of decreased neuroticism can be measured as a decrease in social anxiety using the Brief Fear of Negative Evaluation-revised (BFNE) (Carleton et al., 2006, Depression and Anxiety, 23(5), 297-303; Leary, 1983, Personality and Social Psychology bulletin, 9(3), 371-375). This 12-item Likert scale questionnaire measures apprehension and distress due to concerns about being judged disparagingly or with hostility by others. Ratings use a five-point Likert scale with the lowest, middle, and highest values labeled with “much less than normal,” “normal,” and “much more than normal.” The BFNE can be administered before and repeatedly during therapeutic drug effects. Participants are instructed to answer how they have been feeling for the past hour, or otherwise during the effect of the drug. Baseline-subtracted responses are typically used in statistical models.

The entactogenic effect of authenticity can be measured using the Authenticity Inventory (Kernis & Goldman. 2006. Advances in experimental social psychology, 38, 283-357) as modified by Baggott et al (Journal of Psychopharmacology 2016, 30.4: 378-87). Administration and scoring of the instrument are described by Baggott et al (ibid) and are almost identical to that of the BFNE.

Entactogenic effects on mood can be measured using visual analog scales and other questionnaires common in the clinical research literature (see methods used in Baggott et al., ibid; Holze et al. 2020. Neuropsychopharmacology, 45(3), pp. 462-471; Regan et al. 2021. PloS one, 16(10), p. e025884; Studerus et al. 2021. Journal of Psychopharmacology, p. 0269881121998322 and references therein). Items of particular interest relate to positively valenced experiences and feelings of elation, emotional openness, emotional closeness to others, and love.

Acute physiological changes can be measured in humans with standard clinical methods (blood pressure cuffs, 3-lead EKG, tympanic or oral temperature, serum sodium, etc), with measures usually collected before and at scheduled intervals after an entactogen. For example, measures may be collected before, 0.5, 0.75, 1, 1.5, 2, 2.5, 3, 4, 5, 6, and 8 hours after an entactogen. Maximum change from baseline and area-under-the-effects-versus-time-curve may be used as summary measures and statistically compared to a placebo control condition.

Undesirable symptoms of an entactogen include nausea, vomiting, headache, sedation, difficulty concentrating, lack of appetite, lack of energy, and decreased mood. Additional symptoms are detailed by Vizeli and Liechti (2017. Journal of Psychopharmacology, 31(5), pp. 576-588) and in the MDMA Investigator's Brochure, 14th Edition: Mar. 18, 2022, and references therein, available from the sponsor of MDMA clinical trials at MAPS.org. To measure these and other undesirable effects, patients can be asked to complete a self-report symptom questionnaire, such as the Subjective Drug Effects Questionnaire (SDEQ) or List of Complaints. The SDEQ is a 272-item self-report instrument measuring perceptual, mood, and somatic changes caused by drugs including hallucinogens like LSD (Katz et al. 1968. J Abnorm Psychology 73:1-14). It has also been used to measure the therapeutic and adverse effects of MDMA (Harris et al. 2002. Psychopharmacology, 162(4), 396-405). The List of Complaints is a 66-item questionnaire that measures physical and general discomfort and is sensitive to entactogen-related complaints (e.g., Vizeli & Liechti. 2017. Journal of Psychopharmacology, 31(5), 576-588).

Alternatively, individual items can be taken from the SDEQ or List of Complaints in order to create more focused questionnaires and reduce the burden of filling out time-consuming paperwork on participants.

In some embodiments, a symptom questionnaire is administered approximately 7 hours after a patient takes an entactogen (with instructions to answer for the time since taking the entactogen) and then daily (with instructions to answer for the last 24 hours) for up to 96 hours after the entactogen was taken. Decreases in adverse effects of a formulation compared to standard MDMA can be shown by comparing the intensity or prevalence of effects that occur. Prevalence of adverse effects including headache, difficulty concentrating, lack of appetite, lack of energy, and decreased mood may be decreased by approximately 10%, 20%, 30%, 40%, 50%, 60%, 70%, 80%, 90%, 99%, or 100%.

### Example 14: Evaluation of Therapeutic Profile of Entactogens in Rodents

As an alternative to measuring the therapeutic profile of entactogens in clinical trials, preclinical studies in rodents may also be used. Here, the logic is similar as in the clinical case with desired effects and undesired effects measured. The primary difference is that drug administration typically uses parenteral administration with doses and timing designed to imitate the initial pharmacokinetics of the human case. Thus, rather than, for example, controlled release after oral administration, it makes sense to discuss and evaluate exposure regimens that produce equivalent Tₘₐₓ differences.

Appropriate tasks and behaviors that may be used to measure undesired effects include physiological measures (heart rate, blood pressure, body temperature), the modified Irwin procedure or functional observational battery (Irwin, Psychopharmacologia, 13, 222-257, 1968), and locomotor activity (such as distance traveled, rearing frequency, and rearing duration; Piper et la., J Pharmacol Exp Ther, 317, 838-849, 2006). In these studies, an entactogen is administered at different doses (including a vehicle only placebo) to different groups of animals and measures are made at scheduled times before and after administration. For example, 0, 1.5, 3, 15, and 30 mg/kg of a compound may be administered, and measures made before and 15, 30, 60, 120 and 180 minutes and 12, 24, 36, and 48 hours after administration of the test substance.

The modified Irwin procedure or the functional observational battery (FOB) can be used to compare different exposure profiles to standard exposure profiles (Mathiasen and Moser, 2018. Current protocols in pharmacology, 83(1), p. e43; Redfern et al. 2019. Journal of pharmacological and toxicological methods, 98, p. 106591; Roux, Sable, and Porsolt, 2004. Current protocols in pharmacology, 27(1), pp. 1-23), with an estimated therapeutic index being suggested by the doses that induces marked undesirable nervous system behaviors in comparison to therapeutic doses. Dose ranges are selected to determine a no effect level and lowest observed adverse effect levels.

To estimate effective therapeutic dose, a drug discrimination procedure can be used. Rodent drug discrimination assays are the main methodology for understanding the interoceptive (i.e., felt experience) effects of drugs in animals (Baker, 2017. Neurobiology of Psychedelic Drugs, 201-219; Fantegrossi, Murnane & Reissig. 2008. Biochemical pharmacology, 75(1), 17-33). In a typical discrimination task, an animal is trained to emit one response (often pressing of a lever) during experimental sessions shortly after the administration of a particular drug (the “training drug”), and a different response during sessions that follow administration of the placebo drug vehicle. Once the animals are stably engaged in this task, a novel drug can be tested for similarity to the training drug.

Training and testing procedures are conducted in standard operant conditioning chambers housed in sound-attenuating shells. Dustless Precision Pellets (45 mg; Product #F0021; BioServ, Flemington, NJ) are used as reinforcements for lever pressing.

A training dose of 0.5 to 2.0 mg/kg MDMA is used, and training follows standard procedures (Baker 2017, Neurobiology of Psychedelic Drugs, 201-219). Briefly, rats are trained to discriminate 1.5 mg/kg MDMA from placebo (saline vehicle) under a fixed ratio (FR) 20 schedule of food reinforcement. Lever assignment to stimulus condition is counterbalanced among rats in each experiment. Drug and vehicle training sessions are alternated with the order guaranteeing that the same stimulus condition occurs no more than twice consecutively. The criterion for stimulus control is a minimum of eight out of ten consecutive discrimination training sessions with 80% or higher correct lever responses prior to delivery of the first reinforcer and for the total session.

After stimulus control is established, test sessions are conducted. The test compound is administered to produce the desired release profile prior to starting the test sessions. Test sessions are similar to training sessions, with the exception that responses are not reinforced and sessions end upon completion of the first FR 20 or after 20 min, whichever occurs first. Testing criteria between sessions require subjects to complete at least one drug and at least one vehicle training session consecutively with 80% or higher injection-appropriate responding. The main outcome measure is percent responses on the MDMA-associated lever, while rate of lever pressing (responses per second) is used as a control measure. After a range of doses are administered, it is possible to estimate an EC50 for discrimination. In one embodiment, the EC50 for discrimination is lowered while the lowest observed adverse effect level is unchanged or raised. In another embodiment, the EC50 for discrimination is unchanged while the lowest observed adverse effect level is raised.

While the present invention is described in terms of particular embodiments and applications, it is not intended that these descriptions in any way limit its scope to any such embodiments and applications, and it will be understood that many modifications, substitutions, changes, and variations in the described embodiments, applications, and details of the invention illustrated herein can be made by those skilled in the art without departing from the spirit of the invention, or the scope of the invention as described in the appended claims.

I claim:

1. A pharmaceutical composition comprising MDMA or a pharmaceutically acceptable salt thereof as an immediate release component and a serotonin and norepinephrine reuptake inhibitor (SNRI) as a delayed release component;
wherein:
the SNRI is a compound selected from desvenlafaxine, duloxetine, levomilnacipran, milnacipran, and venlafaxine; or a pharmaceutically acceptable salt thereof.

2. The pharmaceutical composition of claim 1, comprising an enantiomerically enriched mixture of MDMA.

3. The pharmaceutical composition of claim 1, wherein the composition comprises 5-180 mg of MDMA or a pharmaceutically acceptable salt thereof.

4. The pharmaceutical composition of claim 1, wherein the composition comprises 5-90 mg of MDMA or a pharmaceutically acceptable salt thereof.

5. The pharmaceutical composition of claim 3, wherein the SNRI is duloxetine or a pharmaceutically acceptable salt thereof.

6. The pharmaceutical composition of claim 5, wherein the pharmaceutical composition comprises 10-120 mg of duloxetine or a pharmaceutically acceptable salt thereof.

7. The pharmaceutical composition of claim 5, wherein the pharmaceutical composition comprises 5-60 mg of duloxetine or a pharmaceutically acceptable salt thereof.

8. The pharmaceutical composition of claim 3, wherein the SNRI is desvenlafaxine or a pharmaceutically acceptable salt thereof.

9. The pharmaceutical composition of claim 8, wherein the pharmaceutical composition comprises 10-400 mg of desvenlafaxine or a pharmaceutically acceptable salt thereof.

10. The pharmaceutical composition of claim 8, wherein the pharmaceutical composition comprises 5-200 mg of desvenlafaxine or a pharmaceutically acceptable salt thereof.

11. The pharmaceutical composition of claim 3, wherein the SNRI is levomilnacipran or a pharmaceutically acceptable salt thereof.

12. The pharmaceutical composition of claim 11, wherein the pharmaceutical composition comprises 10-120 mg of levomilnacipran or a pharmaceutically acceptable salt thereof.

13. The pharmaceutical composition of claim 11, wherein the pharmaceutical composition comprises 5-60 mg of levomilnacipran or a pharmaceutically acceptable salt thereof.

14. The pharmaceutical composition of claim 3, wherein the SNRI is milnacipran or a pharmaceutically acceptable salt thereof.

15. The pharmaceutical composition of claim 14, wherein the pharmaceutical composition comprises 5-300 mg of milnacipran or a pharmaceutically acceptable salt thereof.

16. The pharmaceutical composition of claim 14, wherein the pharmaceutical composition comprises 2-150 mg of milnacipran or a pharmaceutically acceptable salt thereof.

17. The pharmaceutical composition of claim 3, wherein the SNRI is venlafaxine or a pharmaceutically acceptable salt thereof.

18. The pharmaceutical composition of claim 17, wherein the composition comprises 15-375 mg of venlafaxine or a pharmaceutically acceptable salt thereof.

19. The pharmaceutical composition of claim 17, wherein the composition comprises 5-200 mg of venlafaxine or a pharmaceutically acceptable salt thereof.

20. A method for treating a central nervous system disorder in a human patient comprising administering an effective amount of a pharmaceutical composition of claim 1 to the patient.

21. The method of claim 20, wherein the central nervous system disorder is post-traumatic stress disorder.

22. The method of claim 20, wherein the central nervous system disorder is adjustment disorder.

23. The method of claim 20, wherein the central nervous system disorder is generalized anxiety.

24. The method of claim 20, wherein the central nervous system disorder is social anxiety.

25. The method of claim 20, wherein the central nervous system disorder is a phobia disorder.

26. The method of claim 20, wherein the central nervous system disorder is a substance use disorder.

27. The method of claim 26, wherein the substance use disorder is an alcohol use disorder.

28. The method of claim 20, wherein the central nervous system disorder is an eating disorder.

29. The method of claim 28, wherein the eating disorder is bulimia.

30. The method of claim 28, wherein the eating disorder is binge eating.

31. The method of claim 28, wherein the eating disorder is anorexia.
